(19) United States
(12) Patent Application Publication
(10) Pub. No.: US 2024/0218398 A1
(43) Pub. Date: Jul. 4, 2024

(54) ENGINEERED EXTRACELLULAR VESICLES

(71) Applicant: CORNELL UNIVERSITY, Ithaca, NY (US)

(72) Inventors: Shaoyi JIANG, Ithaca, NY (US); Wenchao GU, Ithaca, NY (US); Sijin LUOZHONG, Ithaca, NY (US); Zhefan YUAN, Ithaca, NY (US)

(73) Assignee: CORNELL UNIVERSITY, Ithaca, NY (US)

(21) Appl. No.: 18/289,191

(22) PCT Filed: May 4, 2022
(86) PCT No.: PCT/US2022/027568

§ 371 Date: Nov. 1, 2023

Related U.S. Application Data
(60) Provisional application No. 63/183,749, filed on May 4, 2021.

(51) Int. Cl. C12N 15/88; C12N 9/22; C12N 15/11; C12N 15/67

(52) U.S. Cl. CPC C12N 15/88; C12N 9/22; C12N 15/11; C12N 15/67; C12N 2310/20; C12N 2510/00

(57) ABSTRACT

Compositions of engineered extracellular vesicles for delivery of cargo to targeted tissues and cells are described herein. Also described herein are methods for making and using the extracellular vesicles described herein. Lastly, described herein are methods for treating a subject, the method comprising administering to a subject an extracellular vesicle as described herein.

### CROSS REFERENCE TO RELATED APPLICATIONS

This application claims the benefit of U.S. Provisional Patent Application No. 63/183,749, filed May 4, 2021, the contents of which is incorporated herein by reference in its entirety.

### INCORPORATION BY REFERENCE OF SEQUENCE LISTING

The Sequence Listing in an ASCII text file, named as 39604WO_9704_02_PC_SequenceListing.txt of 854 KB, created on May 3, 2022, and submitted to the United States Patent and Trademark Office via EFS-Web, is incorporated herein by reference.

### BACKGROUND

Administrating diagnostic or therapeutic agents to a site of interest with precision has presented an ongoing challenge. Currently, two major types of gene therapy delivery systems are viral and non-viral (Y. K. Sung & S. W. Kim, Biomater Res 23, 8 (2019); N. Nayerossadat, et al., Adv Biomed Res 1, 27 (2012)). For viral vectors, safety is the main concern albeit highly effective. Non-viral carriers, including polyplexes and lipoplexes, particularly lipid nanoparticles (LNPs), often suffer from lack of targeting specificity. Recent studies have brought increasing interest in extracellular vesicles (EVs) as a new class of promising carriers for RNA drug delivery, due to their biocompatibility, endogenous function to mediate intercellular exchange of molecules, and native ability to target specific tissues and cross the blood brain barrier (BBB) (Tang et al., Front Oncol 9, 1208 (2019); Aslan et al., BMC Biotechnol 21, 20 (2021); Ridder et al., PLOS Biol 12, e1001874 (2014); Haney et al., J Control Release 207, 18-30 (2015); Alvarez-Erviti et al., Nat Biotechnol 29, 341-345 (2011); Andras & Toborek, Tissue Barriers 4, e1131804 (2016); van Niel et al., Nat Rev Mol Cell Biol 19, 213-228 (2018)). However, the clinical translation of EV-based therapies is hampered by a lack of control over which molecules are loaded from the EV-producing donor cell into the EV. Depending on the donor cell type, the cargo of EVs may include proteins, DNA, RNAs, lipids, nutrients, and metabolic wastes. Unwanted cellular components cannot be excluded from EVs, not only compromising the loading capacity, but also delivering potentially harmful components to the target, such as overexpressed constructs introduced to engineer EVs and cellular waste.

Many systems have been developed to load small RNA cargos, such as siRNAs and microRNAs into EVs, but active enrichment of long mRNAs in EVs remains a challenge (Aslan et al., BMC Biotechnol 21, 20 (2021)). Extremely low copy numbers of endogenous EV-associated RNAs were reported, ranging from 0.02 to 1 RNA per EV and small RNAs are more efficiently packaged into EVs than long mRNAs (0.01 to 1 microRNA vs. 0.001 long intact RNA per EV) (Mosbach et al., Cells 10, (2021); M. Li et al., Philos Trans R Soc Lond B Biol Sci 369, (2014); Chevillet et al., Proc Natl Acad Sci USA 111, 14888-14893 (2014); Z. Wei et al., Nat Commun 8, 1145 (2017)). Only 8% of mRNAs in donor cells may be detected in their EVs (H. Valadi et al., Nat Cell Biol 9, 654-659 (2007)). Previously reported methods loading mRNA into EVs include active and passive encapsulation (X. Luan et al., Acta Pharmacol Sin 38, 754-763 (2017)). For example, Catalase mRNAs were loaded by incubation with macrophage derived EVs after sonication and extrusion, or permeabilization with saponin to treat Parkinson's disease (PD) (Haney et al., J Control Release 207, 18-30 (2015)). To treat leukemia, antisense oligonucleotides (ASOs), CRISPR-Cas9 mRNAs, and guide RNAs (gRNAs) were delivered into EVs originated from enucleated red blood cells (RBCs) by electroporation (W. M. Usman et al., Nat Commun 9, 2359 (2018)). EVs can also be engineered on the parent cell level, with genetic components being introduced to guide the production of designed EVs, often involving extreme overexpression of cargo components to achieve a sufficient loading dose. These procedures may either damage EVs leading to their aggregation or alter the physiology of the EV-producing donor cells, subsequently reducing cargo loading (X. Luan et al., Acta Pharmacol Sin 38, 754-763 (2017); F. Momen-Heravi, et al., Nanomedicine 10, 1517-1527 (2014); J. H. Wang et al., Mol Cancer Ther 17, 1133-1142 (2018)).

Available methods of delivering nucleic acids to cells have well characterized limitations. For example, AAV viral vectors often used for gene therapy are immunogenic, have a limited payload capacity of around 4.4 kb, suffer from poor bio-distribution, can only be administered by direct injection, and pose a risk of disrupting host genes by integration. Non-viral methods have different limitations. Liposomes are primarily delivered to the liver. Extracellular vesicles have limited scalability and purification difficulties. Thus, there is a recognized need for new methods of delivering therapeutic payloads.

Most molecules do not possess inherent affinity in the body. In other cases, the administered agents accumulate either in the liver and the kidney for clearance or in unintended tissue or cell types. Method for improving delivery includes coating the agent of choice with hydrophobic compounds or polymers. Such an approach increases the duration of said agent in circulation and augments hydrophobicity for cellular uptake. On the other hand, this approach does not actively direct cargo to the site of interest for delivery.

To specifically target sites where therapy is needed, therapeutic compounds are optionally fused to moieties such as ligands, antibodies, and aptamers that recognize and bind to receptors displayed on the surface of targeted cells. Upon reaching a cell of interest, the therapeutic compound is optionally further delivered to an intracellular target. For example, a therapeutic RNA can be translated to a protein if it comes into contact with a ribosome in the cytoplasm of the cell.

### SUMMARY

The present disclosure is directed to engineered extracellular vesicles, methods of making engineered extracellular vesicles, and methods of using engineered extracellular vesicles for delivering therapeutic compounds, biologics, or both to tissues and cells of interest.

In a first aspect, the present disclosure is directed to an RNA transcript composition comprising a cargo mRNA which comprises an Arc 5′UTR sequence. In some embodiments, the RNA transcript composition further comprises an Arc mRNA. In some embodiments, the Arc 5′UTR sequence comprises an Arc 5′UTR sequence from a mammal. In some embodiments, the Arc 5′UTR sequence comprises an Arc 5′UTR sequence selected from the group consisting of human, mouse, and rat. In some embodiments, the Arc 5′UTR sequence comprises an Arc 5′UTR sequence from drosophila. In some embodiments, the Arc 5′UTR sequence comprises an Arc 5′UTR sequence having at least 80% sequence identity, at least 85% sequence identity, at least 90% sequence identity, at least 95% sequence identity, at least 98% sequence identity, or at least 99% sequence identity to any one of SEQ ID NOS: 1-4. In some embodiments, the cargo mRNA further comprises a poly(A) signal. In some embodiments, the cargo mRNA encodes a therapeutic protein. In some embodiments, the cargo mRNA encodes a peptide, an enzyme, a cytokine, a hormone, a growth factor, an antigen, an antibody, a portion of an antibody, a clotting factor, a regulatory protein, a signaling protein, a transcription protein, and/or a receptor. In some embodiments, the cargo mRNA encodes a fluorescent protein, a bioluminescent protein, and/or a recombinase reporter. In some embodiments, the Arc mRNA comprises an Arc 3′UTR sequence. In some embodiments, the Arc mRNA comprises an Arc 3′UTR sequence from a mammal. In some embodiments, the mammal is human, mouse, or rat. In some embodiments, the Arc 3′UTR sequence comprises an Arc 3′UTR sequence from drosophila. In some embodiments, the Arc 3′UTR sequence comprises an Arc 3′UTR sequence having at least 80% sequence identity, at least 85% sequence identity, at least 90% sequence identity, at least 95% sequence identity, at least 98% sequence identity, or at least 99% sequence identity to any one of SEQ ID NOS: 5-8. In some embodiments, the Arc mRNA further comprises a poly(A) signal. In some embodiments, the Arc mRNA encodes an Arc protein from a mammal. In some embodiments, the mammal is human, mouse, or rat. In some embodiments, the Arc mRNA encodes an Arc protein from drosophila. In some embodiments, the Arc mRNA comprises an Arc mRNA sequence from a mammal. In some embodiments, the mammal is human, mouse, or rat. In some embodiments, the Arc mRNA comprises an Arc mRNA sequence from drosophila. In some embodiments, the Arc mRNA comprises a nucleotide sequence having at least 80% sequence identity, at least 85% sequence identity, at least 90% sequence identity, at least 95% sequence identity, at least 98% sequence identity, or at least 99% sequence identity to any one of SEQ ID NOS: 9-12.

Some aspects of the disclosure are directed to a recombinant system comprising a DNA encoding a cargo mRNA with an Arc 5′UTR sequence. In some embodiments the recombinant system comprising a DNA encoding a cargo mRNA with an Arc 5′UTR sequence further comprises a second DNA encoding an Arc mRNA. In some embodiments, the Arc 5′UTR sequence comprises an Arc 5′UTR sequence from a mammal. In some embodiments, the Arc 5′UTR sequence comprises an Arc 5′UTR sequence selected from the group consisting of human, mouse, and rat. In some embodiments, the Arc 5′UTR sequence comprises an Arc 5′UTR sequence from drosophila. In some embodiments, the Arc 5′UTR sequence comprises an Arc 5′UTR sequence having at least 80% sequence identity, at least 85% sequence identity, at least 90% sequence identity, at least 95% sequence identity, at least 98% sequence identity, or at least 99% sequence identity to any one of SEQ ID NOS: 1-4. In some embodiments, the cargo mRNA further comprises a poly(A) signal. In some embodiments, the cargo mRNA encodes a therapeutic protein. In some embodiments, the cargo mRNA encodes a peptide, an enzyme, a cytokine, a hormone, a growth factor, an antigen, an antibody, a portion of an antibody, a clotting factor, a regulatory protein, a signaling protein, a transcription protein, and/or a receptor. In some embodiments, the cargo mRNA encodes a fluorescent protein, a bioluminescent protein, and/or a recombinase reporter. In some embodiments, the Arc mRNA comprises an Arc 3′UTR sequence. In some embodiments, the Arc 3′UTR sequence comprises an Arc 3′UTR sequence is from a mammal. In some embodiments, the Arc 3′UTR sequence comprises an Arc 3′UTR sequence is selected from the group consisting of human, mouse, and rat. In some embodiments, the Arc 3′UTR sequence comprises an Arc 3′UTR sequence from drosophila. In some embodiments, the Arc 3′UTR sequence comprises an Arc 3′UTR sequence having at least 80% sequence identity, at least 85% sequence identity, at least 90% sequence identity, at least 95% sequence identity, at least 98% sequence identity, or at least 99% sequence identity to any one of SEQ ID NOS: 5-8. In some embodiments, the Arc mRNA further comprises a poly(A) signal. In some embodiments, the system comprises a single plasmid comprising the DNA encoding a cargo mRNA with an Arc 5′ UTR sequence and the second DNA encoding an Arc mRNA. In some embodiments, the system comprises a first plasmid comprising the DNA encoding a cargo mRNA with an Arc 5′ UTR sequence; and a second plasmid comprising the second DNA encoding an Arc mRNA. In some embodiments, the plasmid(s) further comprises a heterologous DNA regulatory element. In some embodiments, the heterologous DNA regulatory element comprises a promoter, an enhancer, a silencer, an insulator, or combinations thereof. In some embodiments, the Arc mRNA comprises an Arc sequence from a mammal. In some embodiments, the mammal is human, mouse, or rat. the Arc mRNA comprises an Arc sequence from drosophila. In some embodiments, the Arc 5′UTR sequence comprises a nucleotide sequence having at least 80% sequence identity, at least 85% sequence identity, at least 90% sequence identity, at least 95% sequence identity, at least 98% sequence identity, or at least 99% sequence identity to any one of SEQ ID NOS: 1-4. In some embodiments, the Arc mRNA encodes an Arc protein from a mammal. In some embodiments, the mammal is human, mouse, or rat. In some embodiments, the Arc mRNA encodes an Arc protein from drosophila. In some embodiments, the Arc mRNA comprises an Arc mRNA from a mammal. In some embodiments, the mammal is human, mouse, or rat. In some embodiments, the Arc mRNA comprises an Arc mRNA from drosophila. In some embodiments, the Arc mRNA comprises a nucleotide sequence having at least 80% sequence identity, at least 85% sequence identity, at least 90% sequence identity, at least 95% sequence identity, at least 98% sequence identity, or at least 99% sequence identity to any one of SEQ ID NOS: 9-12.

Certain aspects of the current disclosure are directed to an extracellular vesicle comprising an Arc protein; and a cargo mRNA comprising an Arc 5′UTR sequence. Some embodiments are directed to an extracellular vesicle comprising an Arc protein; and a cargo mRNA comprising an Arc 5′UTR sequence wherein the Arc 5′UTR sequence comprises an Arc 5′UTR sequence from a mammal. In some embodiments, the Arc 5′UTR sequence comprises an Arc 5′UTR sequence selected from the group consisting of human, mouse, and rat. In some embodiments, the Arc 5′UTR sequence comprises an Arc 5′UTR sequence from drosophila. In some embodiments, the Arc 5′UTR sequence comprises an Arc 5′UTR sequence having at least 80% sequence identity, at least 85% sequence identity, at least 90% sequence identity, at least 95% sequence identity, at least 98% sequence identity, or at least 99% sequence identity to any one of SEQ ID NOS: 1-4. In some embodiments, the cargo mRNA further comprises a poly(A) signal. In some embodiments, the cargo mRNA encodes a therapeutic protein. In some embodiments, the cargo mRNA encodes a peptide, an enzyme, a cytokine, a hormone, a growth factor, an antigen, an antibody, a portion of an antibody, a clotting factor, a regulatory protein, a signaling protein, a transcription protein, and/or a receptor. In some embodiments, the cargo mRNA encodes a fluorescent protein, a bioluminescent protein, and/or a recombinase reporter. In some embodiments, the Arc protein comprises an Arc protein sequence from a mammal. In some embodiments, the mammal is human, mouse, or rat. In some embodiments, the Arc protein comprises an Arc protein sequence from drosophila. In some embodiments, the Arc protein comprises at least 80% sequence identity, at least 85% sequence identity, at least 90% sequence identity, at least 95% sequence identity, at least 98% sequence identity, or at least 99% sequence identity to any one of SEQ ID NOS: 13-16. In some embodiments, the Arc mRNA comprises an Arc 3′UTR sequence. In some embodiments, the Arc 3′UTR sequence comprises an Arc 3′UTR sequence from a mammal. In some embodiments, the mammal is human, mouse, or rat. In some embodiments, the Arc 3′UTR sequence comprises an Arc 3′UTR sequence from drosophila. In some embodiments, the Arc 3′UTR sequence comprises an Arc 3′UTR sequence having at least 80% sequence identity, at least 85% sequence identity, at least 90% sequence identity, at least 95% sequence identity, at least 98% sequence identity, or at least 99% sequence identity to any one of SEQ ID NOS: 5-8. In some embodiments, the extracellular vesicle further comprises one or more small molecule drugs.

Another aspect of the current disclosure is a method for producing extracellular vesicles, the method comprising: (a) obtaining cells comprising an Arc mRNA and a cargo mRNA which comprises an Arc 5′ UTR; (b) growing the cells in a media under conditions to express an Arc protein encoded by the Arc mRNA, wherein the cells produce extracellular vesicles comprising the Arc protein and the cargo mRNA with the Arc 5′UTR sequence; and (c) isolating the extracellular vesicles from the media. In some embodiments of the method, the cells of step (a) are obtained by introducing into donor cells, a DNA construct which is transcribed into the Arc mRNA and a DNA construct which is transcribed into the cargo mRNA. In some embodiments of the method, the cells of step (a) are obtained by introducing into donor cells, the Arc mRNA and the cargo mRNA. In some embodiments, a recombinant construct is delivered in the form of DNA, RNA, or the combination of both. In some embodiments, the cell is a prokaryotic cell. In some embodiments, the cell is a eukaryotic cell. In some embodiments, the cell is a mammalian cell. In some embodiments, the cell is a human cell. In some embodiments, the donor cells are selected from neural cells, epithelial cells, endothelial cells, hematopoietic cells, connective tissue cells, muscle cells, bone cells, cartilage cells, germline cells, adipocytes, stem cells, self-derived ex vivo differentiated cells, iPSC-derived ex vivo differentiated cells, cancer cells, and combinations thereof. In some embodiments, the donor cells are leukocytes. In some embodiments, the donor cells are self-derived ex vivo differentiated leukocytes. In some embodiments, the donor cells are self-derived ex vivo differentiated monocytes, macrophages, dendritic cells, or combinations thereof. In some embodiments, the donor cells are iPSC-derived ex vivo differentiated leukocytes. In some embodiments, the donor cells are iPSC-derived ex vivo differentiated monocytes, macrophages, dendritic cells, or combinations thereof. In some embodiments, the cells comprising a nucleic acid construct are prepared by transfecting cells with a nucleic acid construct, wherein the transfection is carried out with polyethyleneimine (PEI) complexation, electroporation, cationic lipids complexation, lipid nanoparticle-mediated delivery, microinjection, and combinations thereof.

One aspect of the disclosure is directed to a method for delivering mRNA to a recipient cell, the method comprising: obtaining an extracellular vesicle as described; and contacting the recipient cell with the extracellular vesicle, wherein the extracellular vesicle fuses with the recipient cell, thereby delivering mRNA to the recipient cell. In some embodiments, the contacting is performed in vitro. In some embodiments, the contacting is performed in vivo. In some embodiments, the recipient cell is a mammalian cell. In some embodiments, the recipient cell comprises a hematopoietic cell, a non-hematopoietic cell, a stem cell, or combinations thereof. In some embodiments, the mRNA is delivered to a recipient cell to treat a disease, produce a protein, induce cell death, repress cell death, change cellular ageing, induce immune tolerance, modulate existing immune response, modify intracellular activity, modify cellular behavior, or combinations thereof.

Another aspect of the disclosure is directed to a method for treating a subject in need thereof comprising obtaining extracellular vesicles as described; and administering the extracellular vesicles to the subject. In some embodiments, the extracellular vesicles are administered orally, rectally, intravenously, intramuscularly, subcutaneously, intrauterinely, cerebrovascularly, or intraventricularly. In some embodiments, the extracellular vesicles comprise mRNAs of CRISPR-associated proteins and guide RNAs adapted for treatment of disease including a genetic disorder. Some embodiments are directed to the extracellular vesicles are administered to the subject for treatment of neurodegeneration diseases, aging related disorders, brain tumors, an inflammatory condition, delivering RNAs specifically into inflammatory brain tissues crossing the blood brain barrier without affecting the healthy cells. In some embodiments, the extracellular vesicles are adapted to deliver APOE4 RNA into the brain for the treatment of Alzheimer's disease. In some embodiments, the extracellular vesicles are administered for treatment of cancer, targeting tumor cells without affecting healthy tissues. In some embodiments, the extracellular vesicles comprise mRNA corresponding to tumor associated antigens and wherein the extracellular vesicles are delivered as cancer vaccines for the treatment of cancer, including melanoma, colon cancer, gastrointestinal cancer, genitourinary cancer, hepatocellular cancer. In some embodiments, the extracellular vesicles are delivered for the prevention and/or treatment of infectious diseases. In some embodiments, the extracellular vesicles are delivered for the treatment of autoimmune diseases.

Another aspect of the disclosure is directed to a method to deliver a construct to a recipient cell in vivo to produce an extracellular vesicle as described in vivo using an endogenous Arc. In some embodiments, the vesicle is produced by the endogenous Arc in vivo.

In some embodiments, the construct is delivered in the form of DNA and/or RNA. In some embodiments, the construct is delivered by a lipid nanoparticle, an exosome, a virus, and other gene delivery methods.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1J. Characterization of eaEV that loads and transfers mRNA. (A) The production of eaEV from donor cells and the transduction of eaEV into recipient cells. (B) Fluorescent NTA measures the concentration and size distribution of fluorescently labelled antiArc+ eaEVs among all light scattering EVs (the screen capture shown in C: red circle, eaEV; green circle, non-Arc EV). (D) Each particle's size is plotted as a function of its scattered intensity. (E) The NTA size distribution profile is represented as a histogram of particle concentrations. (F) The percentage of eaEVs in total EVs increased with Arc mRNA transfected, despite that total EV production was also promoted due to liposome transfection alone. (G) A representative NSEM image of eaEV with and without the outer membrane. (H) Fluorescence intensity of total EVs (CMDR+) and eaEVs (anti-Arc+) was measured to quantify their concentrations after purification. Numbers obtained from this fast and easy assay were then corelated to NTA results for the calculation of absolute particle numbers in each sample for future reference. (I) Confocal microscopy observed exosome release via multivesicular body (MVB) membrane fusion, direct outward budding and/or endocytosis of EVs in donor cell culture (blue pixels highlight Arc proteins in EVs whereas green pixels label A5U-GFP cargo mRNAs). (J) The overlap between GFP and CMDR in recipient cells: DAPI+/CMDR− cells without EV uptake showed no GFP signal.

FIGS. 2A-2Q. The addition of A5U significantly improved the efficiency of mRNA encapsulation into eaEV. (A) The 5′UTR of rat Arc shows higher similarity in predicted secondary structure with that of HIV1 than those of mouse and human Arc. The amount of Cy3-A5U-GFP mRNA or Cy5-GFP mRNA loaded into EVs was increased in the presence of Arc capsid, shown by fluorescence intensity reading of purified EVs (B and E), as well as real time epifluorescence imaging of the donor RAW264.7 cell culture (C-D and F-G). (H) RIP followed by RT-qPCR revealed that the addition of A5U increased mRNA loading into Arc capsids. (I-J) Optimization of the ratio of transfection reagents allows both efficient and selective loading of A5U-GFP cargo compared to 18S, GAPDH and rArc. The cargo A5U-GFP mRNA, capsid Arc mRNA, capsid Arc protein and nuclei in the recipient cells were stained and imaged, with a total number of 614 cells and 1611 EVs analyzed, with a representative area shown in (K) and magnified views in (L, M-O). Circles highlight Arc proteins indicating individual eaEV. With a ratio, Arc:A5U-GFP=1.5:1, almost all Arc (protein)+ EVs overlapped with A5U-GFP mRNAs (M-O, green & yellow), but none with Arc mRNAs (M-O, red). In addition to being loaded into Arc EVs, A5U-GFP and Arc mRNAs can also be seen in other EVs (L, red & yellow). GFP protein can be packed into EVs with an extremely low frequency compared to the mRNA cargo (L, M-O, purple). The percentage of colocalization between capsid protein and the GFP mRNA was quantified and compared to Arc mRNA (P-Q). No overlap between Arc protein and Arc mRNA was observed in extracellular vesicles.

FIGS. 3A-3G. The addition of A5U significantly improved the efficiency and stability of mRNA delivery into recipient cells. (A1-A2) Upon EV transduction, real-time live-cell imaging of Cy3 fluorescence was performed at 15-min (B1-B4), 1-hr, 4-hr (C1-C4), 3-day, 6-day and 12-day time points to quantify cargo mRNA up-taken by recipient cells. Transfer by eaEV carrying A5U-GFP showed a highly stable and drastic increase in the amount of cargo accumulated in recipient cells (A1), whereas Arc EV carrying cargo without the A5U appeared less stable and less efficient (A2). (D) First, CMDR (cell mask deep red) membrane dye was used to label total EVs, enabling fast and accurate quantification of total EVs in buffer as well as in recipient cells. Titration experiments suggested the best dye concentration of 1:5000, for moderate fluorescent signal with individual EVs visible in recipient cells. This dye concentration was then used to optimize the appropriate amount of EVs transferred to recipient cells, with a linear correlation demonstrated between CMDR signal and EV concentrations. (E) We added the same amount of EVs from each group to recipient cells, calculated with their CMDR fluorescence. After 2 hours, recipient cells receiving EVs from different groups showed similar levels of CMDR fluorescence, suggesting an uptake of similar numbers of total EVs. (F) Arc/A5U-GFP showed a consistent increase in cargo translation among control groups at 4 hours and 1 day after the EV transduction. On day 3, recipient cells started to show strong autofluorescence in control groups and the increase by Arc/A5U-GFP was no longer significant. (G) The percentage of recipient cells expressing GFP was low but significantly increased with Arc and A5UGFP.

FIGS. 4A-4J. A5U-eaEV enriches cargo mRNA in the aged brain. (A) BM cells are extracted from the mouse femur and cultured for 7 days in complete medium supplemented with GM-CSF and IL4 to differentiate into monocytes, dendritic cells, and macrophages, from which control and engineered EVs are produced and injected intravenously into mouse models for neuroinflammation. (B) Morphology of representative GM-CSF/IL4 BM cultures at day 6, bright filed image. (C) Phenotype of representative GM-CSF/IL4 BM cultures at day 6. CD11c+ MHCII+BMDCs (C, top left) can be sub-divided based on CD11b and MHCII expression (C, bottom). Boxes depict gates and numbers correspond to percentage of cells in each gate. Histograms showing surface expression of the indicated markers by MHCIIhighCD11blow and MHCIIintCD11bhigh subsets. (D) Day 6 GM-CSF/IL4 BM cells were able to uptake its own EVs labelled by CMDR. (E1-E4) After the transfection of mRNA transcripts, EVs were produced for 40 hours before collection and purification. This long production led to the saturation of EVs in the supernatant culture medium, yielding roughly an equal number of total EVs from each sample group (F). Meanwhile, the proportion of eaEV among total EVs is significantly higher in the Arc/A5U-GFP group. Purified EVs (1× and 2× dilutions) were stained with CMDR and a fluorescent Arc antibody, whose epifluorescence intensity was measured for EV concentrations. (G) Representative IVIS images show the in vivo biodistribution of Cy3+A5U-GFP mRNAs, which were significantly enriched in the aged brain with systemic administration by eaEVs, compared to the no Arc A5U-GFP only control. The photo overlay of radiance is displayed, with the color range from 3.3e+7 to 4.9e+8 and a color threshold at 3.5e+8, to subtract the background signal based on the negative control animals. In addition to representative images shown here, the mock transfection control, the PBS (no EV) injection control, and CMDR+ animals were also analyzed as the negative control for the Cy3 IVIS imaging here. (H) IVIS imaging of the in vivo biodistribution of Cy5+ GFP mRNA suggest that mRNA cargo does not enrich in the aged brain by eaEV delivery without the A5U motif added to the cargo construct. Like in (G), a color threshold of 3.5e+8 was applied to subtract the background signal based on the negative control animals (Cy3+Cy5− Arc/A5U-GFP and A5U-GFP groups). (I-J) Quantification of the IVIS signal. Mean+SD, n=2 for experimental groups; n=7 for NC groups. A sample size of 6 per group was used based on the power analysis with an 80% power, a 5% significance level, and a tow-sided t-test following Cohen's power analysis.

FIGS. 5A-5G. BM-DC/M derived A5U-eaEV can deliver mRNA into neurons across the BBB targeting chronic pan-neuronal inflammation. (A, A′, B, B′, where A′ and B′ are enlargements of A and B) A5U-GFP mRNA was successfully delivered across the BBB to express GFP proteins in the inflammatory aged brain: white pixels (GFP+/NeuN+) highlight colocalization between GFP and NeuN-Alexa647, representing neuronal GFP expression. In contrast, green pixels showed GFP in non-neuron cells, likely infiltrated immune cells. Magnified views of the hypothalamus are shown in (C-D). (E-F) Two days and even six days after the IV injection, aged brains showed a significant increase in the level of GFP expression in NeuN+ cells. Systemic injection of eaEV/A5U-GFP led to comparable levels of GFP expression in infiltrated peripheral immune cells cells (green pixels, C-E), but a significant enrichment in NeuN+ neurons in aged brains (white pixels, C-E). (G) The integrated expression of GFP among control and experimental groups in various brain regions. Certain brain regions, such as the arcuate hypothalamic nucleus (ARH), medial preoptic nucleus (MPN), ventral tegmental area (VTA), showed more significant increases than other regions including the hippocampus and the prefrontal cortex (PFC).

FIGS. 6A-6H. BM-DC/M derived A5U-eaEV can deliver mRNA into neurons across the BBB targeting acute ischemic stroke injury. In the acute ischemic stroke model (A), eaEV specifically delivered GFP into neurons in the stroke area (B-C) without affecting the control regions (B-C′). Magnified views (D-E) showed GFP expression in many NeuN+ neurons (white arrows) and a small number of Iba1+ microglia/macrophages, with further magnification in (F-G), which also showed many NeuN+ and Iba1+ cells without any GFP expression suggesting that the GFP signal observed is specific. There is clearly also GFP expression in NeuN−/Iba1− cells. (H) Compared to the control area, the number of NeuN+ neurons decreased in the stroke area, whereas that of Iba1+ immune cells increased (microglia and infiltrated macrophages). The number of GFP-expressing cells increased. All GFP+ cell count in the control area is non-specific background signal from autofluorescence in the microvessels.

FIGS. 7A-7E. Transfection of engineered DNA constructs and RNA transcripts into donor cells to test their functionality. (A) DNA constructs and RNA transcripts to encode the cargo have been verified in HEK293 and RAW 264.7 cells with a random mutation negative control, a distinct fluorophore mCherry control, and mock transfection controls, with live-cell epifluorescence imaging applied to monitor expression in real time lapse. (B) RAW264.7 donor cell expression at 8-hour post transfection. (C) RAW264.7 donor cell expression at 24-hour post transfection. (D-E) The number and viability of donor cells at the end of EV production was always recorded and compared to ensure a good and comparable quality of EVs produced among control and experimental groups.

FIGS. 8A-8C. Optimization of RNA transfection and EV production titrations with real-time live-cell imaging. (A) Shows real-time live-cell imaging of transfected donor cells at 6 doses of lipofectamine. (B) Graphs cell number in 96-well plate 4 hours post transfection. (C) compares cell number in a 6 well plate at 24 hours post transfection and 96 hours post transfection. The conclusion is that too much mRNA with not enough lipofectamine led to low viability of donor cells. Optimal dose was decided to be 100 ng total mRNA transfected per 20,000 donor cells (96-well, 100 μL total opti-MEM medium with 0.3 μL lipofectamine per well).

FIGS. 9A-9B. Optimization of the ratio between capsid and cargo mRNA transfection components. (A) Detailed optimization of the transfection ratio between capsid and cargo mRNAs was carried out in RAW264.7 cells. Lowest ratio of 0 Arc: 0 GFP is shown in A1 while ratio of 3:3 Arc:GFP is shown in (A16). (B) Graphically shows that with an increase in the amount of capsid Arc mRNA transfected, more cargo A5U-GFP mRNA was encapsulated instead of being translated, leading to decreased GFP protein expression. It was decided to not only focus on an amount of cargo mRNA leading to a high level of GFP expression when transfected alone, but also with the capsid mRNA introduced, a significant decrease in GFP expression compared to having the cargo alone. The decided ratio was 3 Arc: 2 A5U-GFP (or 1.5 Arc: 1 A5U-GFP) shown in A12.

FIG. 10. Visualization and optimization of RNA encapsulation. (A) real-time live-cell imaging of transfected cells using Cy3 and Cy5. (B) graphical representation of fluorescence intensity reading of RNA encapsulation.

FIG. 11. DNA transfection time lapse with quantification of GFP expression. With PEI-DNA transfection into HEK293 cells, since there is a stable source for continuing production of both capsid and cargo mRNAs and proteins, we observed a significant and stable increase in GFP expression transfecting both cargo and capsid. Eventually GFP expression in donor cells reached saturation (by 24 hours).

FIGS. 12A-12M. EV characterization and EV production optimization. (A-C) NTA results suggested that the production of total EVs was comparable among all transfected control and experimental groups. (K-M) More larger vesicles were produced with Arc transfection, likely being Arc ectosomes. (D-H) Adding the Arc capsid and A5U-GFP cargo enabled the most significant secretion of Arc EVs, compared to other control groups including Arc/GFP. (H) A large proportion of all EVs in Arc/A5U-GFP were Arc ectosomes. (I) Serum free production of EVs led to a higher-level secretion of mouse CD63+ EVs and a larger proportion of eaEVs among all EVs secreted. (J) EVs can be stored at 4° C. for a short period of time and then started aggregation.

FIG. 13. CMDR dye optimization to be used for the quantification of EV uptake. Staining with a plasma membrane stain, CMDR, was carried out to facilitate the quantification of EVs in recipient cells. The number of EVs to be transferred to recipient cells was also optimized.

FIGS. 14A-14B. Total EV (CMDR+) biodistribution at day 3 after IV injection. With the same total number of EVs injected into each control or experimental mouse (per kg body weight), the biodistribution of total EVs 3 days post IV injection (with EVs mostly degraded) was similar in all organs collected. Total EVs were labeled by a plasma membrane dye, CMDR.

FIG. 15A-15E. eaEV can deliver mRNA into the tumor. (A) In vivo biodistribution of CMDR+ total EVs in control and experimental groups: (1) no EV negative control (NC); (2) mock transfection (noTrans) EV control; (3) Arc negative EV carrying either GFP or A5U-GFP mRNAs; (4) Arc positive EV loaded with GFP or A5U-GFP mRNAs. Photo overlay of radiance was shown with a color range of 3.3e+07 to 5.0e+09 and a threshold at 3.5e+08. (B) Quantification of the CMDR biodistribution in mice injected with leukocyte eaEVs. (C) High resolution CLSM images showed high levels of GFP expression in the tumor but minimal expression in other organs. Whole-tumor imaging (D-E) with K-Ras staining (D1-E1) and a magnified view of regions deep into the tumor further away from the big vessels (D2-E3) suggest that eaEV facilitates deep tumor penetration and mRNA delivery.

FIG. 16. eaEV can load small molecule drugs into MB231 triple negative breast cancer cells. SM drug1 was loaded into the donor cells producing eaEVs at 1:500 concentration and such eaEVs were able to deliver this fluorescently labelled drug into recipient cells (top), whereas 1:50000 drug loaded was too diluted to transfer the drugs as a negative control (bottom).

FIG. 17 shows a map of an example DNA plasmid used to prepare the capsid for engineered EVs.

FIG. 18A-18B. The mechanism of selective cargo loading and the structure of an example carrier. (A) Arc 5′UTR enables the recognition of a specific cargo mRNA by the Arc capsid protein, while Arc 3′UTR accelerates nonsense-mediated mRNA decay of the Arc capsid mRNA after its translation. Altogether these allow the selective loading of cargo without the interference of overexpressed capsid mRNA. (B) An example structure of engineered EVs containing Arc protein capsids and nucleic acid cargos.

### DETAILED DESCRIPTION

Natural nanocarriers, extracellular vesicles (EVs), are a promising new class of drug carriers due to their biocompatible nature and endogenous functions that mediate long range intercellular exchange of molecules, along with a native ability to deliver to desired targets. Meanwhile, therapeutic messenger RNAs (mRNAs) have gained increasing interest in recent years. However, efficient and selective encapsulation of long mRNA into EVs remains problematic. Disclosed herein, the virus-like but retrotransposon Arc protein capsid is incorporated in the lumen of EV (“Arc EV”). Arc EV possesses high effectiveness like a viral vector and biocompatibility as a naturally occurring vesicle. Arc EV also plays native roles in loading and transferring mRNA inter-neuronally, making it a great tool for mRNA delivery into the brain, among other target tissues and cells of interest. The disclosed engineered Arc EV (eaEV) further enables highly efficient and stable encapsulation of specific mRNA cargo. Naturally equipped with donor cell's homing molecules for the neuroinflammatory microenvironment, leukocyte-derived eaEV facilitates efficient delivery of mRNA into disease neurons across the blood brain barrier. This disclosure provides a novel endogenous virus-like system capable of loading and delivering specific mRNA to target tissues and cells of interest.

Enabled by the incorporation of a virus-like protein capsid that binds to an RNA motif included in a cargo mRNA, engineered Arc EV has high mRNA cargo loading and transduction efficiency. Immunologically inert eaEV can be produced from various types of cells, including monocyte-derived cells, to deliver mRNA across the blood brain barrier (BBB), specifically targeting the neuroinflammatory microenvironment in vivo, which demonstrates the therapeutic potential of this nanoscale, biocompatible, and efficient mRNA drug carrier.

### Extracellular Vesicle

The terms “extracellular vesicle (EV)” or “vesicle” as used herein, refer to a cell-derived vesicle that is generated by a combination of endocytotic and exocytotic events that result in the encapsulation of various biomolecules. All prokaryotic and eukaryotic cells release EVs as part of their normal physiology and during acquired abnormalities. While EVs can be broadly divided into two categories, ectosomes and exosomes, the terms “ectosomes,” “exosomes,” and “EVs” may be used interchangeably for purposes of this disclosure. Ectosomes are vesicles that pinch off the surface of the plasma membrane via outward budding, and include microvesicles, microparticles, and large vesicles in the size range of ˜50 nm to 1 μm in diameter. Exosomes are EVs with a size range of ˜40 to 160 nm (average ˜100 nm) in diameter with an endosomal origin. Such encapsulation may protect a therapeutic nucleic acid from enzymatic degradation or other environmental stresses (e.g., ionic strength, pH etc.). The association of proteins with an EV provides stability in both extracellular and intracellular environments as well as facilitates a cell-targeting mechanism for cell-cell communication.

In some embodiments, EVs may be created in prokaryotes, eukaryotes, or viruses. In some embodiments, the engineered EVs are made in yeast, bacteria, virus, protists, or other types of cells, whether they be unicellular organisms or multicellular organisms, or non-living items which contain DNA.

Exosomes are produced by many different types of cells, including immune cells such as B lymphocytes, T lymphocytes, dendritic cells (DCs) and most cells. Exosomes are also produced, for example, by glioma cells, platelets, reticulocytes, neurons, intestinal epithelial cells and tumor cells. Exosomes for use in the disclosed compositions and methods can be derived from any suitable cell, including the cells identified above. Exosomes have also been isolated from physiological fluids, such as plasma, urine, amniotic fluid and malignant effusions. Non-limiting examples of suitable exosome producing cells for mass production include dendritic cells (e.g., immature dendritic cell), Human Embryonic Kidney 293 (HEK) cells, 293T cells, Chinese hamster ovary (CHO) cells, and human ESC-derived mesenchymal stem cells.

In some embodiments, exosomes are derived from DCs, such as immature DCs. Exosomes produced from immature DCs do not express MHC-II, MHC-I or CD86. As such, these exosomes do not stimulate naive T cells to a significant extent and are unable to induce a response in a mixed lymphocyte reaction allowing exosomes produced from immature dendritic cells to be good candidates for use in delivery of genetic material.

Exosomes can also be obtained from any autologous patient-derived, heterologous haplotype-matched or heterologous stem cells as to reduce or avoid the generation of an immune response in a patient to whom the exosomes are delivered. Any exosome-producing cell can be used for this purpose.

Exosomes produced from cells can be collected from the culture medium by any suitable method. Typically, a preparation of exosomes can be prepared from cell culture or tissue supernatant by centrifugation, filtration or combinations of these methods. For example, exosomes can be prepared by differential centrifugation, that is low speed (<20000 g) centrifugation to pellet larger particles followed by high speed (>100000 g) centrifugation to pellet exosomes, size filtration with appropriate filters (for example, 0.22 uin filter), gradient ultracentrifugation (for example, with sucrose gradient) or a combination of these methods.

The disclosed exosomes may be administered to a subject by any suitable means. Administration to a human or animal subject may be selected from parenteral, intramuscular, intracerebral, intravascular, subcutaneous, or transdermal administration. In some embodiments, the method of delivery is by injection. Preferably the injection is intramuscular or intravascular (e.g. intravenous). A physician will be able to determine the required route of administration for each patient in need of therapy.

The exosomes are preferably delivered as a composition. The composition may be formulated for parenteral, intramuscular, intracerebral, intravascular (including intravenous), subcutaneous, or transdermal administration. Compositions for parenteral administration may include sterile aqueous solutions which may also contain buffers, diluents and other suitable additives. The exosomes may be formulated in a pharmaceutical composition, which may include pharmaceutically acceptable carriers, thickeners, diluents, buffers, preservatives, and other pharmaceutically acceptable carriers or excipients and the like in addition to the exosomes.

In some embodiments of the current disclosure, the representative composition of the engineered EVs comprises cell membrane components (e.g., structural lipids and membrane proteins), Arc proteins or protein motifs (e.g., the MA or CA domains of Arc), enriched RNAs, and potentially other cargo components (e.g., proteins, RNAs, DNAs, nutrients, metabolites, and bioactive compounds). Engineered Arc EVs enrich desired cargo RNAs and minimize the packaging of unwanted cellular components.

Some embodiments of the current disclosure comprise an extracellular vesicle composition comprising an Arc protein and a cargo mRNA comprising an Arc 5′UTR sequence.

In some embodiments of the extracellular vesicle composition, the Arc protein binds to the Arc 5′UTR sequence of the cargo mRNA. The binding facilitates the packaging of the cargo mRNA into the exosomes. In some embodiments, the Arc 5′UTR sequence improves loading of the cargo mRNA into the EV with at least 25% improved loading. In some embodiments, the Arc 5′UTR sequence improves loading of the cargo mRNA into the EV with at least 50% improved loading. In some embodiments, the Arc 5′UTR sequence improves loading of the cargo mRNA into the EV with at least 75% improved loading. In some embodiments, the Arc 5′UTR sequence improves loading of the cargo mRNA into the EV with at least 1000% improved loading. In some embodiments, the Arc 5′UTR sequence improves loading of the cargo mRNA into the EV with at least 150% improved loading. In some embodiments, the Arc 5′UTR sequence improves loading of the cargo mRNA into the EV with at least 200% improved loading. In some embodiments, the Arc 5′UTR sequence improves loading of the cargo mRNA into the EV with at least 250% improved loading. In some embodiments, the Arc 5′UTR sequence improves RNA transduction into recipient cells. In some embodiments, the Arc 5′UTR sequence improves RNA transduction into recipient cells with at least 25% improved transduction over cargo mRNA without the Arc 5′UTR sequence. In some embodiments, the Arc 5′UTR sequence improves RNA transduction into recipient cells with at least 50% improved transduction. In some embodiments, the Arc 5′UTR sequence improves RNA transduction into recipient cells with at least 75% improved transduction. In some embodiments, the Arc 5′UTR sequence improves RNA transduction into recipient cells with at least 100% improved transduction. In some embodiments, the Arc 5′UTR sequence improves RNA transduction into recipient cells with at least 125% improved transduction. In some embodiments, the Arc 5′UTR sequence improves RNA transduction into recipient cells with at least 150% improved transduction.

In some embodiments, the EV composition further comprises one or more small molecules. In some embodiments, the EV is emersed in a drug solution comprising a desired small molecule. In some embodiments, the drug solution contains a desired small molecule at a predetermined concentration. The EVs then intake the small molecule in a passive transport process. In some embodiments, the small molecule is placed into the EV through physical means, such as sonication where the membrane of the EV is manipulated allowing the small molecule entry into the lumen of the EV.

By “small molecule,” it is meant a low molecular weight organic compound that may regulate a biological process. Small molecules typically have a molecular weight of at least 100 g/mol, 200 g/mol, or 500 g/mol, 1000 g/mol, 2000 g/mol, and up to 5,000 g/mol, 10,000 g/mol, 20,000 g/mol, 50,000 g/mol, or 100,000 g/mol (e.g., 100-50,000 g/mol, 100-10,000 g/mol). Many pharmaceuticals are small molecules and are called small molecule drugs. As used herein, small molecules and small molecule drugs can be used interchangeably.

Some examples of a small molecule are insulin, aspirin, and antihistamines. In some embodiments, small molecules include biological molecules such as fatty acids, glucose, amino acids, and cholesterol as well as secondary metabolites such as lipids, glycosides, alkaloids, and natural phenols. In some embodiments, small molecules are used to treat neurological diseases. In some embodiments, small molecules are used to treat autoimmune disorders. In some embodiments, small molecules are chemotherapeutic agents or anticancer drugs. In some embodiments, small molecules are inhibitors that target tyrosine kinase cell surface receptors or intracellular serine/threonine kinases involved in cellular signaling pathways such as the PI3K/Akt/mTOR signaling. In some embodiments, the small molecules are inhibitors which target apoptotic proteins, epigenetic regulators, and other proteins to deregulate cancer cell growth.

### Arc

Arc (activity-regulated cytoskeleton-associated protein) regulates the endocytic trafficking of a-amino-3-hydroxy-5-methylisoxazole-4-propionic acid (AMPA) type glutamate receptors. Arc activities have been linked to synaptic strength and neuronal plasticity. Phenotypes of loss of Arc in experimental murine model included defective formation of long-term memory and reduced neuronal activity and plasticity.

Arc exhibits similar molecular properties to retroviral Gag proteins. There appears to be a structural and functional relationship between Arc and retroviral Gag polyprotein. Arc was identified in a computational search for domesticated retrotransposons harboring Gag-like protein domains. Arc contains structural elements found within viral Group-specific antigen (Gag) polyproteins that may have originated from the Ty3/gypsy retrotransposon family (Campillos et al., Trends Genet. 22:585-589, 2006; Shepherd, Semin. Cell Dev. Biol. 77, 73-78, 2018; Zhang et al., Neuron 86, 490-500, 2015). Biochemical studies showed that mammalian Arc has a positively charged N-terminal domain (NTD) and a negatively charged C-terminal domain (CTD), separated by a flexible linker (Myrum et al., Biochem. J. 468, 2015). Crystal structure analysis of the isolated CTD revealed two lobes, both with striking 3D homology to the capsid (CA) domain of HIV Gag (Zhang et al., 2015). In retroviruses, self-association of CA allows assembly of Gag polyproteins into the immature capsid shell (Lingappa et al., Virus Res. 193, 89-107, 2014; Perilla and Gronenborn, Trends Biochem. Sci. 41, 410-420, 2016). Remarkably, recombinant Arc from fruit fly and rat was subsequently shown to self-assemble into spheroid particles with resemblance to HIV Gag capsids (Ashley et al., 2018; Pastuzyn et al., Cell 172, 275-288.e18, 2018). The Arc capsids are released in extracellular vesicles and capable of transmitting RNA cargo to recipient cells (Ashley et al., Cell 172, 262-274, 2018; Pastuzyn et al., 2018). These studies implicate Arc as an endogenous neuronal retrovirus, and oligomeric assembly of Arc into virus-like capsids mediates the capture and intercellular transfer of RNA (Parrish and Tomonaga, Cell 172, 8-10, 2018; Shepherd, 2018).

In some embodiments, Arc is a non-human Arc polypeptide. In some embodiments, the Arc polypeptide comprises a full-length Arc polypeptide (e.g., a full-length non-human Arc polypeptide). In other embodiments, the Arc polypeptide comprises a fragment of non-human Arc, such as a truncated Arc polypeptide, that participates in the formation of a capsid. In additional embodiments, the Arc polypeptide comprises one or more domains of a non-human Arc polypeptide, in which at least one of the domains participates in the formation of a capsid. In further embodiments, the Arc polypeptide is a recombinant Arc polypeptide.

In some embodiments, the Arc polypeptide is a human Arc polypeptide with at least its RNA binding domain modified to bind to a cargo that is not native to the human Arc. In some embodiments, the Arc polypeptide comprises a full-length human Arc polypeptide with at least its RNA binding domain modified to bind to a cargo that is not native to the human Arc protein. In other embodiments, the Arc polypeptide comprises a human Arc fragment comprising modification(s) in at least its RNA binding domain. In additional embodiments, the Arc polypeptide comprises one or more domains of a human Arc polypeptide, in which at least one of the domains participates in the formation of a capsid and in which the RNA binding domain is modified to bind to a cargo that native human Arc protein does not bind to. In further embodiments, the Arc polypeptide is a recombinant human Arc polypeptide, with at least the RNA binding domain is modified to enable loading of a cargo that is not native to the human Arc protein.

The various domains of Arc polypeptides have been described in the art. See, e.g., Pastuzyn et al., Cell 172, 275-288.e18, 2018 where highly conserved, unique orthologs of the murine Arc genes were identified throughout the tetrapods (mammals, birds, reptiles, amphibians) or Hallin et al., Biochemistry And Biophysics Reports, 26, 100975, 2021. For example, the domain of a human Arc polypeptide that participates in the formation of a capsid comprises amino acids 205-364 of a human Arc polypeptide, such as the human Arc polypeptide as reported in GenBank under Accession No. 23237 (SEQ ID NO: 13). Arc polypeptides from other species, such as mammalian species, can be utilized as well.

In some embodiments, the Arc polypeptide comprises an amino acid sequence that is at least 80% identical, at least 85% identical, at least 90% identical, at least 95% identical, at least 98% identical, or at least 99% identical, to SEQ ID NO: 13. In some embodiments, the Arc polypeptide comprises the amino acid sequence of SEQ ID NO: 13. In some embodiments, the Arc polypeptide comprises an amino acid sequence that is at least 80% identical, at least 85% identical, at least 90% identical, at least 95% identical, at least 98% identical, or at least 99% identical, to SEQ ID NO: 14. In some embodiments, the Arc polypeptide comprises the amino acid sequence of SEQ ID NO: 14. In some embodiments, the Arc polypeptide comprises an amino acid sequence that is at least 80% identical, at least 85% identical, at least 90% identical, at least 95% identical, at least 98% identical, or at least 99% identical, to SEQ ID NO: 15. In some embodiments, the Arc polypeptide comprises the amino acid sequence of SEQ ID NO: 15. In some embodiments, the Arc polypeptide comprises an amino acid sequence that is at least 80% identical, at least 85% identical, at least 90% identical, at least 95% identical, at least 98% identical, or at least 99% identical, to SEQ ID NO: 16. In some embodiments, the Arc polypeptide comprises the amino acid sequence of SEQ ID NO: 16.

### Arc Capsid

Arc monomers oligomerize into viral capsids. Arc spontaneously forms oligomeric structures that resemble virus-like capsids. Purified preparations of rat Arc capsids exhibited a double-shell structure with a mean diameter of 32±0.2 nm (Pastuzyn et al., Cell 172, 275-288 e218 (2018)). Similarly, bacterially-expressed and purified dArc1, a Drosophila Arc homologue, also self-assembled into capsid-like structures. Purified Arc protein that was expressed in an insect cell expression system also assembled into similar virus-like capsids, all of which shows that Arc oligomerization is not an artifact of bacterial expression. Immature retroviral capsids are formed by the uncleaved Gag polyprotein, and the major stabilizing interactions are made by the C-terminal domain (CTD) of the CA region (Mattei et al., Science 354, 1434-1437 (2016)). Drosophila homologs of Arc have shown viral like behavior, auto-assembling structures of the homologs closely matched that of HIV-1 and Ty3 capsids (Erlendsson et al., Nat Neurosci. 23, 172-175 (2020)). Consistent with other viral capsids, both drosophila Arc homologs formed pentamers and hexamers, which together framed the capsid shell. Also, the homologs formed protrusions on the surface of the capsid. (Budnik and Thomson, Nature Neuroscience.). Arc protein capsid naturally enriches mRNA into the lumen of EV, favoring the loading of mRNAs over other cellular components such as DNA, proteins, metabolic waste etc. Arc protein oligomerizes to form a capsid encapsulating Arc mRNAs. In the absence of endogenous Arc mRNA, Arc protein capsid could package and transfer other abundant RNAs (Pastuzyn et al., Cell 172, 275-288 e218 (2018)).

Arc mRNA

By an “Arc mRNA” it is meant an mRNA coding for an Arc polypeptide as described herein. In some embodiments, the Arc mRNA comprises a 5′ untranslated region (UTR) that is an Arc 5 ‘UTR. In some embodiments the Arc mRNA comprises a 3′UTR that is an Arc 3’ UTR. In some embodiments, the Arc mRNA is chimeric in that it comprises an Arc 5′ UTR, Arc mRNA coding sequence that encodes an Arc polypeptide, and an Arc 3′UTR where two or all of the three sequences are heterologous, i.e., from different species. In some embodiments, the Arc mRNA comprises an Arc 5′ UTR, Arc mRNA coding sequence that encodes an Arc polypeptide, and an Arc 3′UTR that are all from the same species.

In some embodiments, the Arc mRNA does not comprise a 5′UTR such as an Arc 5′ UTR. In some embodiments, an Arc mRNA includes a 5′ UTR. In some embodiments, an Arc mRNA comprises a 5′UTR that is not an Arc 5′ UTR. In some embodiments, an Arc mRNA does not comprise a 5′ UTR.

In some embodiments, an Arc mRNA does not comprise a 3′UTR such as an Arc 3′ UTR. In some embodiments, an Arc mRNA comprises a 3′UTR such as an Arc 3′ UTR. In some embodiments, an Arc mRNA comprises a 3′UTR that is not an Arc 3′UTR sequence. In some embodiments, an Arc mRNA does not comprise a 3′ UTR.

In some embodiments, the Arc mRNA is an mRNA encoding an Arc polypeptide from a mammal. In some embodiments, the Arc mRNA is an mRNA encoding a human Arc polypeptide. In some embodiments, the Arc mRNA is an mRNA encoding a non-human Arc polypeptide. In some embodiments, the Arc mRNA is an mRNA encoding a mouse Arc polypeptide. In some embodiments, the Arc mRNA is an mRNA encoding a rat Arc polypeptide.

In some embodiments, an Arc mRNA is an mRNA encoding an Arc polypeptide from a non-mammal. In some embodiments, the Arc mRNA is an mRNA encoding a drosophila Arc polypeptide.

In some embodiments, the Arc mRNA comprises a sequence that is at least 80% identical, at least 85% identical, at least 90% identical, at least 95% identical, at least 98% identical, or at least 99% identical to SEQ ID NO: 9. In some embodiments, the Arc mRNA comprises nucleic acid SEQ ID NO: 9. In some embodiments, the Arc mRNA comprises a sequence that is at least 80% identical, at least 85% identical, at least 90% identical, at least 95% identical, at least 98% identical, or at least 99% identical to SEQ ID NO: 10. In some embodiments, the Arc mRNA comprises nucleic acid SEQ ID NO: 10. In some embodiments, the Arc mRNA comprises a sequence that is at least 80% identical, at least 85% identical, at least 90% identical, at least 95% identical, at least 98% identical, or at least 99% identical to SEQ ID NO: 11. In some embodiments, the Arc mRNA comprises nucleic acid SEQ ID NO: 11. In some embodiments, the Arc mRNA comprises a sequence that is at least 80% identical, at least 85% identical, at least 90% identical, at least 95% identical, at least 98% identical, or at least 99% identical to SEQ ID NO: 12. In some embodiments, the Arc mRNA comprises nucleic acid SEQ ID NO: 12.

In some embodiments, the Arc mRNA comprises a poly(adenylation) signal.

In some embodiments, the Arc mRNA comprises an Arc 3′UTR sequence.

### Arc 5′UTR

Arc and Gag were reported to show little specificity for a particular mRNA in vitro without their 5′ untranslated region (UTR) (Ashley et al., Cell 172, 262-274 e211 (2018); Comas-Garcia, et al., Viruses 8, (2016).). “Arc 5′UTR (A5U)” as used herein means a 5′UTR of a naturally occurring Arc mRNA. This is a region that is not translated into a protein.

As described above, in some embodiments, the 5′UTR is optional for an Arc mRNA. For example, in some embodiments, an Arc mRNA includes a 5′ UTR; in other embodiments, an Arc mRNA does not comprise a 5′ UTR; in other embodiments, an Arc mRNA comprises a 5′UTR that is not an Arc 5′ UTR; and in other embodiments, an Arc mRNA does not comprise a 5′UTR at all.

In embodiments where an Arc mRNA comprises an A5U, the A5U is an A5U sequence from a mammal. In further embodiments, the A5U is from a human. In other embodiments, the A5U is from mouse. In other embodiments, the A5U is from rat. In some embodiments, the A5U is from drosophila.

In some embodiments, an A5U is added to the cargo construct. The addition of an A5U to the cargo construct enabled high cargo loading efficacy. 3′UTR

In some embodiments, the Arc 5′UTR comprises a sequence that is at least 80% identical, at least 85% identical, at least 90% identical, at least 95% identical, at least 98% identical, or at least 99% identical, to SEQ ID NO: 1. In some embodiments, the Arc 5′UTR comprises SEQ ID NO: 1. In some embodiments, the Arc 5′UTR comprises a sequence that is at least 80% identical, at least 85% identical, at least 90% identical, at least 95% identical, at least 98% identical, or at least 99% identical, to SEQ ID NO: 2. In some embodiments, the Arc 5′UTR comprises SEQ ID NO: 2. In some embodiments, the Arc 5′UTR comprises a sequence that is at least 80% identical, at least 85% identical, at least 90% identical, at least 95% identical, at least 98% identical, or at least 99% identical, to SEQ ID NO: 3. In some embodiments, the Arc 5′UTR comprises SEQ ID NO: 3. In some embodiments, the Arc 5′UTR comprises a sequence that is at least 80% identical, at least 85% identical, at least 90% identical, at least 95% identical, at least 98% identical, or at least 99% identical, to SEQ ID NO: 4. In some embodiments, the Arc 5′UTR comprises SEQ ID NO: 4.

### Arc 3′ UTR

The term “3′ UTR sequence” as used herein, refers to an mRNA-derived 3′ untranslated repeat sequence that is capable of binding to a protein within an extracellular vesicle. For example, an Arc 3 ‘UTR sequence may bind to an Arc protein within an extracellular vesicle. Such 3’ UTR binding to a protein may occur with only the 3′ UTR sequence, or when the 3′ UTR sequence is linked to a non-Arc nucleic acid.

In some embodiments, an Arc mRNA does not comprise a 3′UTR sequence. In other embodiments, an Arc mRNA includes a 3′UTR sequence. In other embodiments, an Arc mRNA includes a 3′UTR sequence that is an Arc 3′UTR sequence. In some embodiments, an Arc mRNA comprises a 3′UTR that is not an Arc 3′UTR sequence.

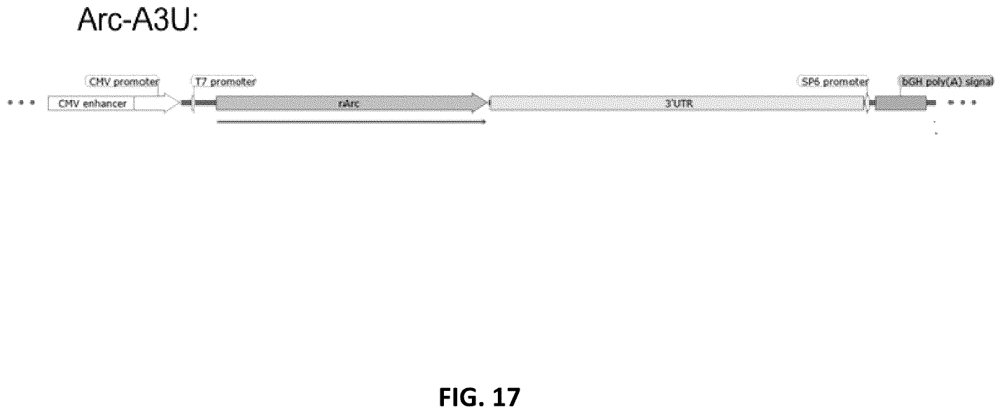

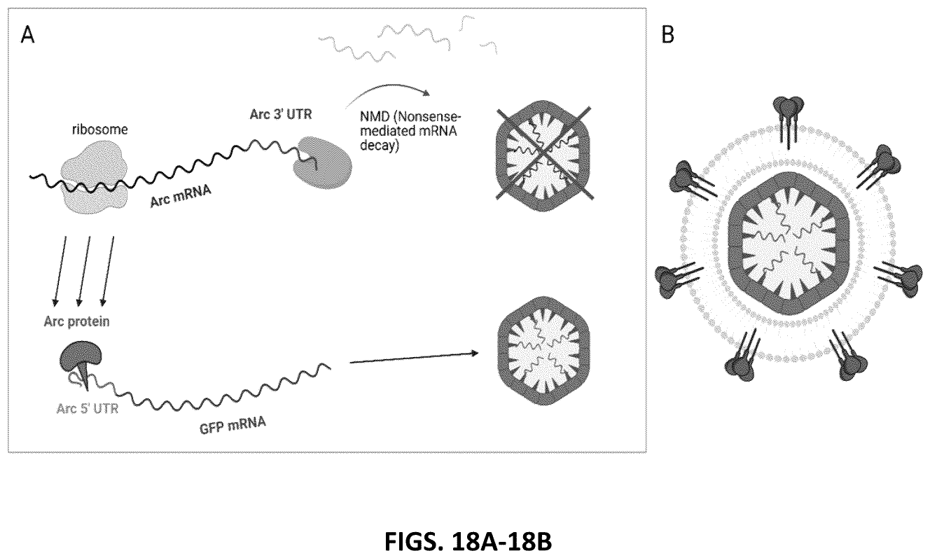

In some embodiments of the present disclosure, modifications of the capsid Arc gene are presented to enable the fast clearance of Arc mRNAs after they are translated into proteins. In some embodiments, such modification is achieved by the addition of a rat Arc 3′UTR sequences (A3U, partial or full) using various molecular cloning techniques. An Arc 3′UTR can be from human, mouse, rat (NCBI Gene IDs #23237, #11838, #54323) or drosophila (dArc1, NCBI Gene ID #36595). These embodiments provide the map of an exemplary DNA plasmid cloned to generate an exemplary capsid for engineered EVs (FIG. 17). Fast removal of the Arc mRNA after translation avoids overexpression of Arc in target cells without compromising the production of the carrier (FIG. 18A). The disclosure further includes the addition of A3U sequences to a sequence encoding the full-length Arc protein, Arc protein motifs, and any codon optimized sequences of these motifs from all species. Arc mRNA sequences with and without an A3U sequence are both included in the present disclosure.

In some embodiments, the Arc 3′UTR is mammalian. In further embodiments, the Arc 3′UTR is human. In other embodiments, the Arc 3′UTR is from mouse. In other embodiments, the Arc 3′UTR is from rat. In some embodiments, the Arc 3′UTR is not mammalian. In further embodiments, the Arc 3′UTR is from drosophila.

In some embodiments, the Arc 3′UTR comprises a nucleic acid sequence that is at least 80% identical, at least 85% identical, at least 90% identical, at least 95% identical, at least 98% identical, or at least 99% identical, to SEQ ID NO: 5. In some embodiments, the Arc 3′UTR mRNA comprises SEQ ID NO: 5. In some embodiments, the Arc 3′UTR comprises a sequence that is at least 80% identical, at least 85% identical, at least 90% identical, at least 95% identical, at least 98% identical, or at least 99% identical, to SEQ ID NO: 6. In some embodiments, the Arc 3′UTR comprises SEQ ID NO: 6. In some embodiments, the Arc 3′UTR comprises a sequence that is at least 80% identical, at least 85% identical, at least 90% identical, at least 95% identical, at least 98% identical, or at least 99% identical, to SEQ ID NO: 7. In some embodiments, the 3′ Arc UTR comprises SEQ ID NO: 7. In some embodiments, the Arc 3′UTR comprises a sequence that is at least 80% identical, at least 85% identical, at least 90% identical, at least 95% identical, at least 98% identical, or at least 99% identical, to SEQ ID NO: 8. In some embodiments, the Arc 3′UTR comprises SEQ ID NO: 8.

### Cargo

In some embodiments, a composition of the disclosure (for example, an arc EV) comprises a cargo. In some embodiments, the Arc EVs comprise a cargo mRNA. As used herein, the term “cargo mRNA” refers to any nucleic acid that is not a portion of, or transcribed from, the arc gene. In some embodiments, the cargo mRNA encodes a therapeutic protein. In some embodiments, the cargo mRNA encodes a peptide, an enzyme, a cytokine, a hormone, a growth factor, an antigen, an antibody, a portion of an antibody, a clotting factor, a regulatory protein, a signaling protein, a transcription protein, and/or a receptor. In some embodiments, the cargo mRNA encodes a reporter protein. In some embodiments, the cargo mRNA encodes a fluorescent protein, a bioluminescent protein, and/or a recombinase reporter. In some embodiments, the cargo mRNA comprises a combination of a therapeutic protein and a reporter protein.

In some embodiments, a cargo mRNA comprises an Arc 5′ UTR. In such embodiments, the cargo mRNA and the Arc 5′UTR sequences are designed so that the sequence is one contiguous sequence starting with an upstream Arc 5′UTR followed by the coding portion of the cargo mRNA sequence for a desired protein. In some embodiments, the cargo mRNA is a chimeric mRNA where its only 5′UTR is an Arc 5′UTR and its coding portion is a non-Arc coding portion.

In some embodiments, the cargo mRNA does not comprise a 3′UTR sequence. In some embodiments, the cargo mRNA comprises a 3′UTR sequence that is not an Arc 3′UTR sequence. In some embodiments, the cargo mRNA comprises a 3′UTR sequence. In some embodiments, the cargo mRNA comprises a 3′UTR sequence that is an Arc 3′UTR sequence.

In some embodiments, the nucleic acid molecule is an RNA polymer, e.g., a single stranded RNA polymer, a double stranded RNA polymer, or a hybrid of single and double stranded RNA polymers. In some embodiments, the RNA comprises and/or encodes an antisense oligoribonucleotide, a siRNA, an mRNA, a tRNA, an rRNA, a snRNA, a shRNA, microRNA, or a non-coding RNA.

In some embodiments, the nucleic acid molecule comprises a hybrid of DNA and RNA.

In some embodiments, the nucleic acid molecule is an antisense oligonucleotide, optionally comprising DNA, RNA, or a hybrid of DNA and RNA.

In some embodiments, the nucleic acid molecule comprises and/or encodes an RNAi molecule. In some embodiments, the RNAi molecule is a microRNA (miRNA) molecule. In other embodiments, the RNAi molecule is an siRNA molecule. The miRNA and/or siRNA are optionally double-stranded or as a hairpin, and further optionally encapsulated as precursor molecules.

In some embodiments, the nucleic acid molecule is for use in a nucleic acid-based therapy. In some embodiments, the nucleic acid molecule is for regulating gene expression (e.g., modulating mRNA translation or degradation), modulating RNA splicing, or RNA interference. In some cases, the nucleic acid molecule comprises and/or encodes an antisense oligonucleotide, microRNA molecule, siRNA molecule, mRNA molecule, for use in regulation of gene expression, modulating RNA splicing, or RNA interference.

In some embodiments, the nucleic acid molecule is for use in gene editing. Exemplary gene editing systems include, but are not limited to, CRISPR-Cas systems, zinc finger nuclease (ZFN) systems, and transcription activator-like effector nuclease (TALEN) systems. In some embodiments, the nucleic acid molecule comprises and/or encodes a component involved in the CRISPR-Cas systems, ZFN systems, or the TALEN systems.

In some embodiments, the cargo is a therapeutic agent. In some embodiments, the cargo is a small molecule, a protein, a peptide, an antibody or binding fragment thereof, a peptidomimetic, or a nucleotidomimetic. In some embodiments, the cargo is a therapeutic cargo, comprising e.g., one or more drugs. In some embodiments, the cargo comprises a diagnostic tool, for profiling, e.g., one or more markers (such as markers associates with one or more disease phenotypes). In additional embodiments, the cargo comprises an imaging tool.

In some embodiments, the nucleic acid molecule is for use in antigen production for therapeutic and/or prophylactic vaccine production. For example, the nucleic acid molecule encodes an antigen that is expressed and elicits a desirable immune response (e.g., a pro-inflammatory immune response, an anti-inflammatory immune response, an B cell response, an antibody response, a T cell response, a CD4+ T cell response, a CD8+ T cell response, a Th1 immune response, a Th2 immune response, a Th17 immune response, a Treg immune response, or a combination thereof).

In some embodiments, the nucleic acid molecule comprises a nucleic acid enzyme.

Nucleic acid enzymes are RNA molecules (e.g., ribozymes) or DNA molecules (e.g., deoxyribozymes) that have catalytic activities. In some embodiments, the nucleic acid molecule is a ribozyme. In other embodiments, the nucleic acid molecule is a deoxyribozyme. In some cases, the nucleic acid molecule is a MNAzyme, which functions as a biosensor and/or a molecular switch (see, e.g., Mokany, et al., JACS 132(2): 1051-1059 (2010)). Some embodiments of the current disclosure comprise an RNA transcript composition comprising an Arc mRNA as well as a cargo mRNA with an Arc 5′UTR sequence.

### Vectors

Some embodiments of the current disclosure comprise a recombinant system comprising a first DNA encoding an Arc mRNA and a second DNA encoding a cargo mRNA with an Arc 5′UTR sequence. In some embodiments, the recombinant system comprises a single construct comprising the first DNA and second DNA. In some embodiments, the recombinant system comprises a first construct comprising the first DNA and a second construct comprising the second DNA. In some embodiments, the constructs further comprise a heterologous DNA regulatory element, where a “DNA regulatory element” a DNA sequence that certain transcription factors recognize and bind to in order to recruit or repel RNA polymerase. In further embodiments, the heterologous DNA regulatory element comprises a promoter, an enhancer, a silencer, an insulator, or combinations thereof.

Each of the first nucleic acid sequence and the second nucleic acid sequence can be operably inserted into an expression vector. In some embodiments, the first nucleic acid sequence and second nucleic acid sequence are operably inserted in a common expression vector and they are expressed together. For example, in some embodiments, the second nucleic acid encoding the chimeric polynucleotide is inserted in frame into an intron of the first nucleic acid encoding the Arc protein. Methods to construct expression vectors containing genetic sequences and appropriate transcriptional and translational control elements are well known in the art. These methods include in vitro recombinant DNA techniques, synthetic techniques, and in vivo genetic recombination. Such techniques are described in Sambrook et al., Molecular Cloning, A Laboratory Manual (Cold Spring Harbor Press, Plainview, N.Y., 1989), and Ausubel et al, Current Protocols in Molecular Biology (John Wiley & Sons, New York, N.Y., 1989).

Vectors include but are not limited to plasmids, viral nucleic acids, viruses, phage nucleic acids, phages, cosmids, and artificial chromosomes. Expression vectors generally contain regulatory sequences necessary elements for the translation and/or transcription of the inserted coding sequence. For example, the coding sequence is preferably operably linked to a promoter and/or enhancer to help control the expression of the desired gene product. Promoters used in biotechnology are of different types according to the intended type of control of gene expression. They can be generally divided into constitutive promoters, tissue-specific or development-stage-specific promoters, inducible promoters, and synthetic promoters.

Depending on the vector system and the host utilized, any number of suitable transcription and translation elements may be used. In mammalian cell systems, promoters from mammalian genes or from mammalian viruses are preferable.

### Methods of Making Arc Extracellular Vesicles

Some embodiments of the current disclosure comprise a method of making EVs comprising obtaining cells comprising an Arc mRNA and a cargo mRNA which comprises an Arc 5′ UTR; growing the cells in a media under conditions to express an Arc protein encoded by the Arc mRNA, wherein the cells produce extracellular vesicles comprising the Arc protein and the cargo mRNA with the Arc 5′UTR sequence; and isolating the extracellular vesicles from the media. In some embodiments, the method of making EVs comprises obtaining cells comprising obtaining cells comprising an Arc mRNA and a cargo mRNA which comprises an Arc 5′ UTR, by introducing into donor cells, a DNA construct which is transcribed into the Arc mRNA and a DNA construct which is transcribed into the cargo mRNA. In some embodiments, the method of making EVs comprises obtaining cells comprising obtaining cells comprising an Arc mRNA and a cargo mRNA which comprises an Arc 5′ UTR, by introducing into donor cells, by introducing into donor cells, the Arc mRNA and the cargo mRNA.

“Donor cells” as used herein is a term of art. The donor cells function in that the construct is inserted into the donor cell, and the cell produces the EV. For example, when the Arc mRNA and the cargo mRNA comprising an Arc 5′UTR are inserted into the donor cell, by methods known and described below, the donor cell translates the Arc mRNA to synthesize the Arc polypeptide. The Arc polypeptide then forms the EV through its retrovirus like budding mechanism—much like the retrovirus like budding mechanism of Gag. Since all cells make EVs, all cells can be donor cells.

In some embodiments, EVs are created in prokaryotes, eukaryotes, or viruses. In some embodiments, the engineered EVs are made in yeasts, bacteria, viruses, protists, or other types of cells, whether they be unicellular organisms, multicellular organisms, or non-living items which contain DNA.

The donor cells may provide the EV with its targeting specificity. This is due to the native ability of EVs from different donor cell types in targeting various tissues.

In some embodiments, the method of making EVs further comprises donor cells that are selected from neural cells, epithelial cells, endothelial cells, hematopoietic cells, connective tissue cells, muscle cells, bone cells, cartilage cells, germline cells, adipocytes, stem cells, self-derived ex vivo differentiated cells, iPSC-derived ex vivo differentiated cells, cancer cells, and combinations thereof. In further embodiments, the donor cells are leukocytes. In some embodiments, the donor cells are self-derived ex vivo differentiated leukocytes. In some embodiments, the donor cells are self-derived ex vivo differentiated monocytes, macrophages, dendritic cells, or combinations thereof. In some embodiments, the donor cells are iPSC-derived ex vivo differentiated leukocytes. In some embodiments, the donor cells are iPSC-derived ex vivo differentiated monocytes, macrophages, dendritic cells, or combinations thereof.

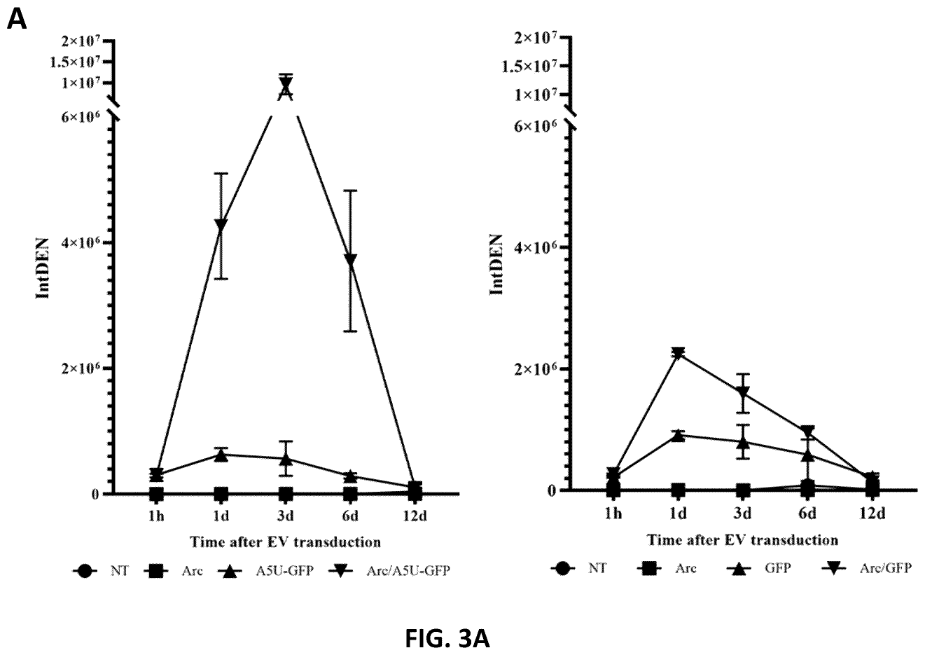

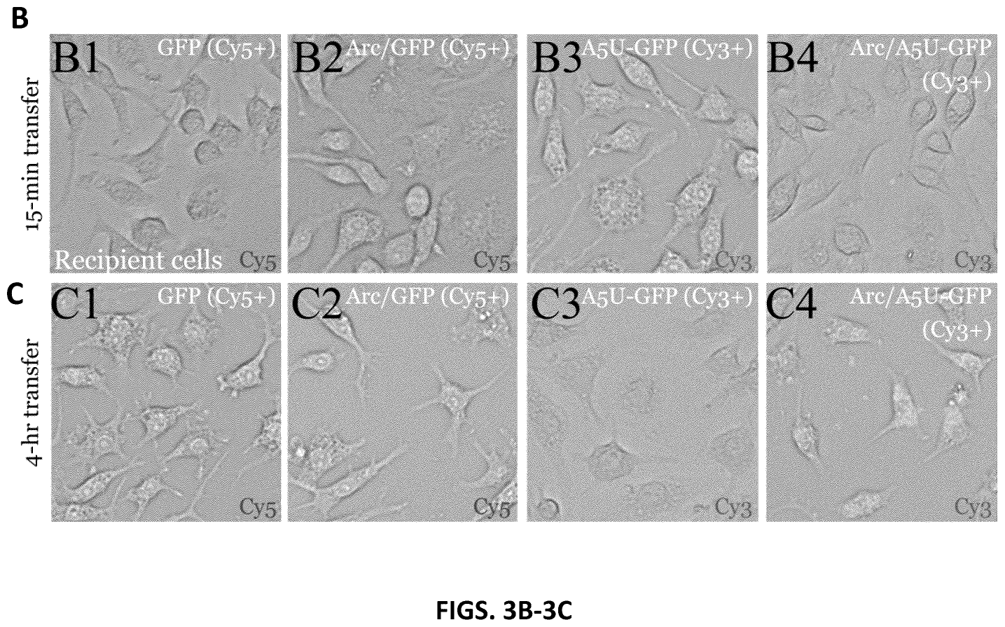

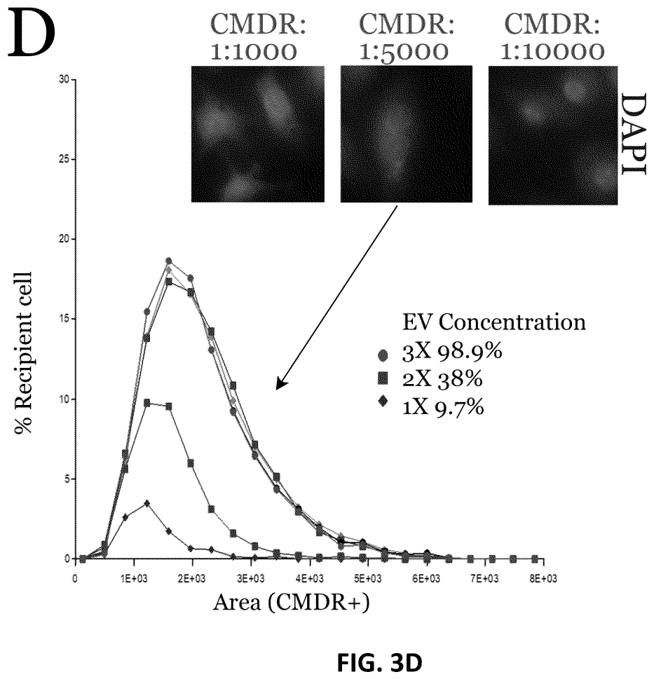

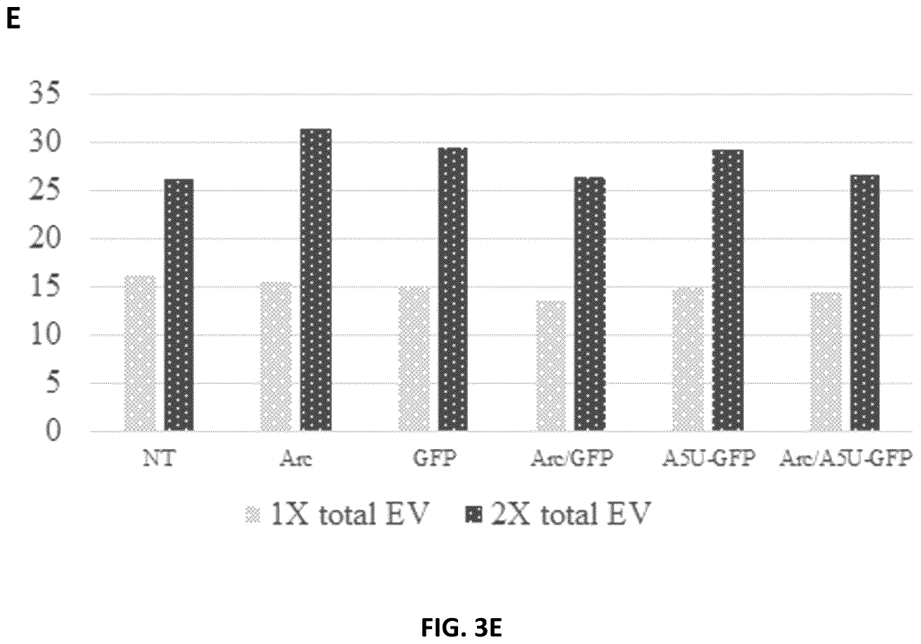

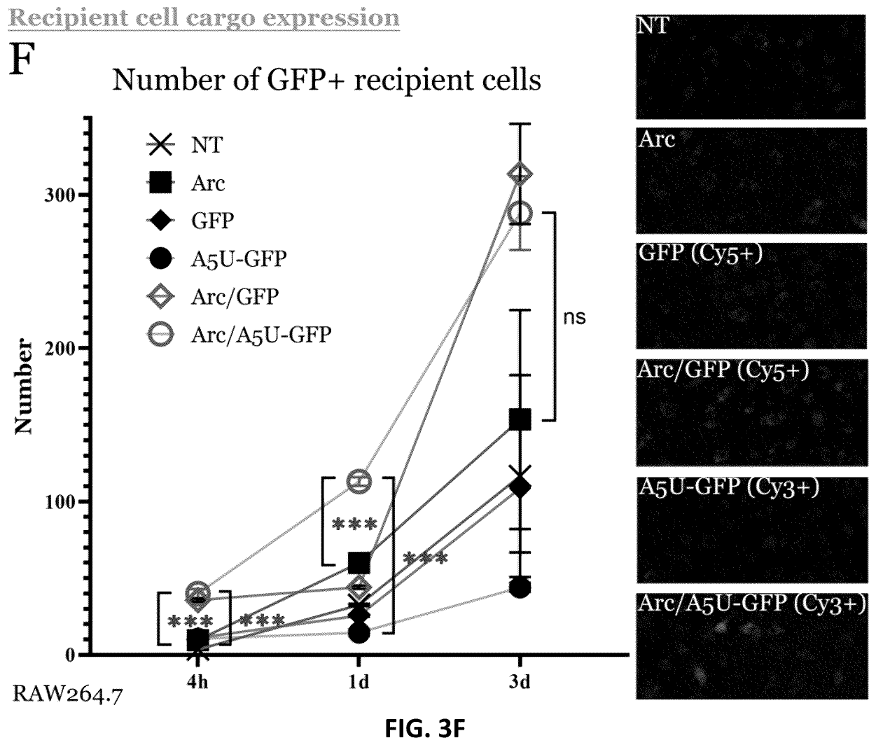

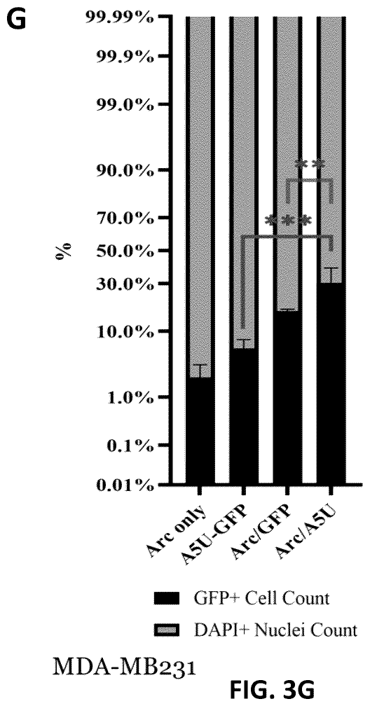

Creating the donor cells occurs through the placing of the two nucleic acids, the Arc mRNA and the cargo mRNA attached to the A5U, into the donor cell. This placing can be done through methods known in the art, including, but not limited to, physical methods such as direct micro injection, biolistic particle delivery, electroporation, sonoporation, and laser-based optical transfection; and chemical methods, such as calcium phosphate, cationic polymer, lipofection, fugene, or dendrimer transfection. In some embodiments, the transfer of nucleic acids into to the donor cells takes place through polyethyleneimine (PEI) complexation, electroporation, cationic lipids complexation, lipid nanoparticle-mediated delivery, microinjection, or through the use of adenoviral vectors. While the cargo mRNA does not need to be attached to the A5U, RNA transduction into recipient cells is less efficient and less stable without the A5U included as is seen in FIG. 3A2.

### Applications

Some aspects of the current disclosure include a method for delivering mRNA to a recipient cell. In some embodiments, the method comprises obtaining an extracellular vesicle as described herein and contacting the extracellular vesicle with a recipient cell, wherein the extracellular vesicle fuses with the cell, thereby delivering the desired mRNA to the recipient targeted cell. In some embodiments, the method of delivering mRNA to the recipient cell is performed in vitro. In some embodiments, the method of delivering mRNA to the recipient cell is performed in vivo as described below.

In some embodiments of the current disclosure, the mRNA is delivered to a recipient cell to treat a disease, produce a protein, induce cell death, repress cell death, change cellular ageing, induce immune tolerance, modulate existing immune response, modify intracellular activity, modify cellular behavior, or combinations thereof.

The eaEV of the present disclosure can be applied in a wide range of therapeutics. In some embodiments, the EVs are used for the treatment for cancer. In some embodiments, the EVs are used to prevent and/or treat viral infection. In some embodiments, the EVs are used in the treatment and/or prevention of allergies. In some embodiments, the EVs are used to treat tissue degeneration. In some embodiments, the EVs are used to treat inflammatory diseases. For example, EVs from peripheral immune cells can cross the blood brain barrier under inflammatory conditions to deliver drugs into disease cells without affecting healthy tissues. Such EVs can also deliver drugs preferably into inflammatory microenvironments. In further embodiments, the EVs deliver the therapeutic to tumor inflammatory microenvironments. In some embodiments, the target of such EVs include virally infected tissues for the treatment of virus infection. In other embodiments, the EVs include cargo mRNA that can be used for gene therapy.

Some aspects of the current disclosure include methods for treating a subject with extracellular vesicles. In some embodiments, the method for treating a subject with EVs comprises obtaining EVs and administering to a subject in need thereof. In some embodiments, the EVs are administered orally, rectally, intravenously, intramuscularly, subcutaneously, intrauterinely, cerebrovascularly, or intraventricularly. In some embodiments, the EVs comprise mRNAs of CRISPR-associated proteins and guide RNAs adapted for treatment of disease including a genetic disorder. In some embodiments, the extracellular vesicles are administered for treatment of neurodegeneration diseases, aging related disorders, brain tumors, inflammatory conditions, delivering RNAs specifically into inflammatory brain tissues crossing the blood brain barrier without affecting the healthy cells. In some embodiments, the EVs comprise mRNA corresponding to tumor associated antigens and wherein the extracellular vesicles are delivered as cancer vaccines for the treatment of cancer, including melanoma, colon cancer, gastrointestinal cancer, genitourinary cancer, hepatocellular cancer. In some embodiments, the EVs are delivered to prevent contraction of infectious disease, i.e. vaccination. In some embodiments, the EVs are delivered for the treatment of autoimmune diseases. A non-limiting example of treatment of an autoimmune disease is to deliver the mRNA of interleukin-1 receptor antagonist (IL-1ra), or the recombinant version, anakinra, for the treatment of rheumatoid arthritis, an autoimmune disease in which IL-1 plays a key role.

Another aspect of the current disclosure is a method to deliver the construct of the cargo linked to the A5U to a donor cell in vivo to produce an EV in vivo using endogenous Arc protein. In some embodiments, the construct is delivered as DNA. In some embodiments, the construct is delivered as RNA. In some embodiments, the construct is delivered to the donor cell by a lipid nanoparticle, an exosome, a virus, or another gene delivery method.

### EXAMPLES

The following examples are presented to illustrate the present disclosure. The examples are not intended to be limiting in any manner.

### Example 1. Engineering, Production, and Isolation of eaEV to Load and Deliver mRNA

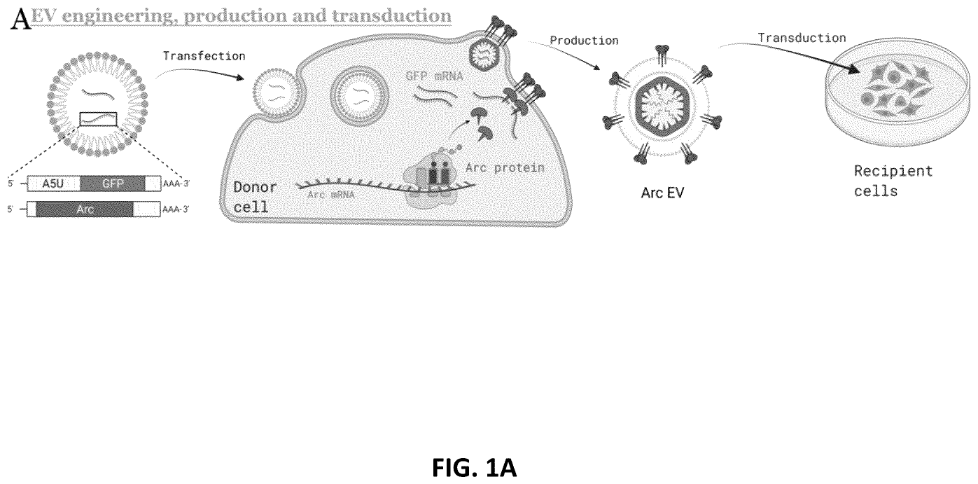

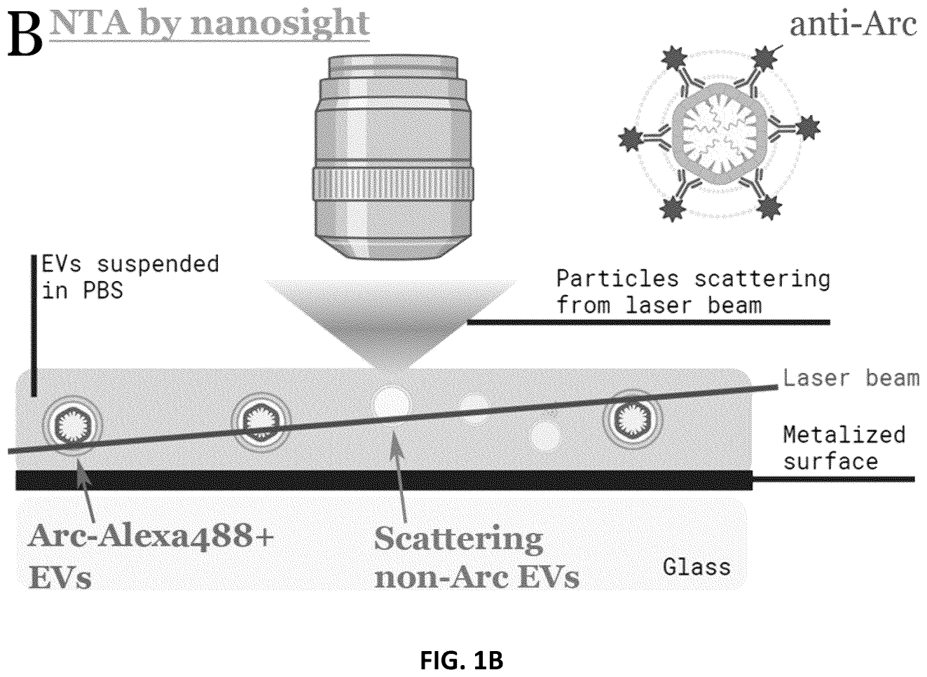

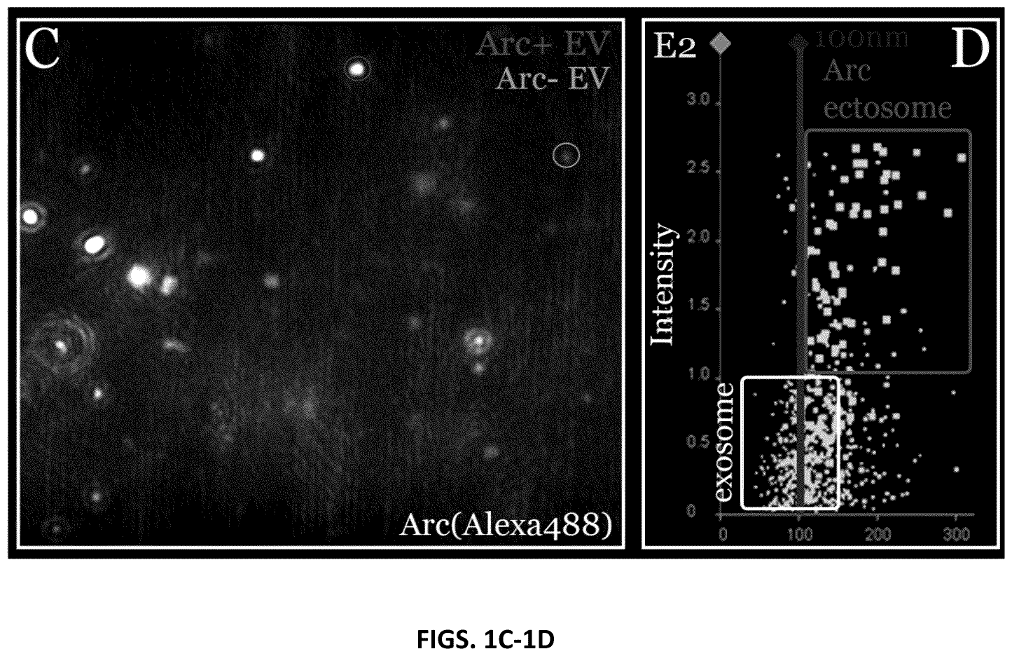

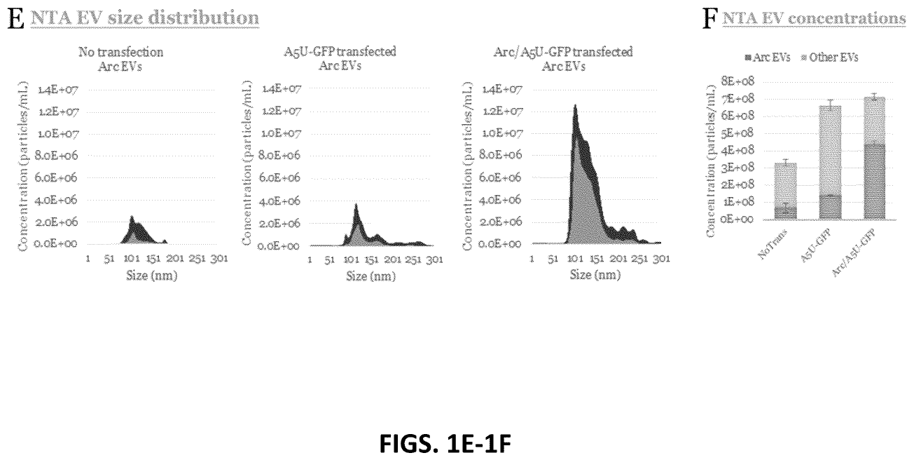

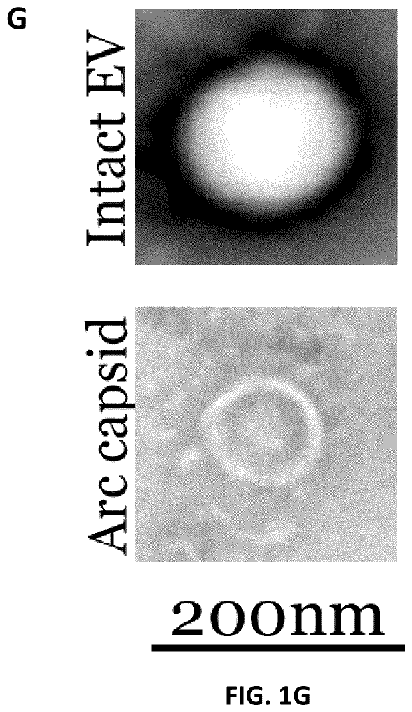

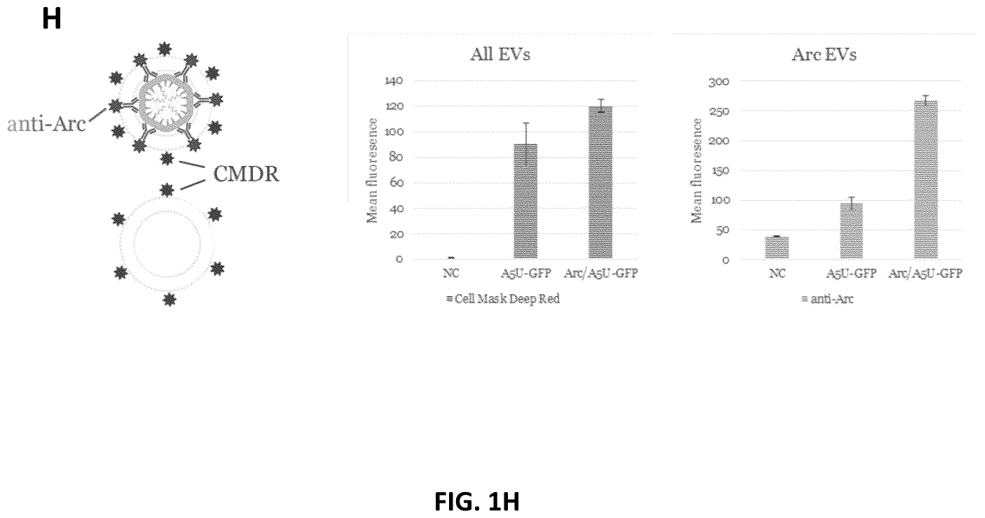

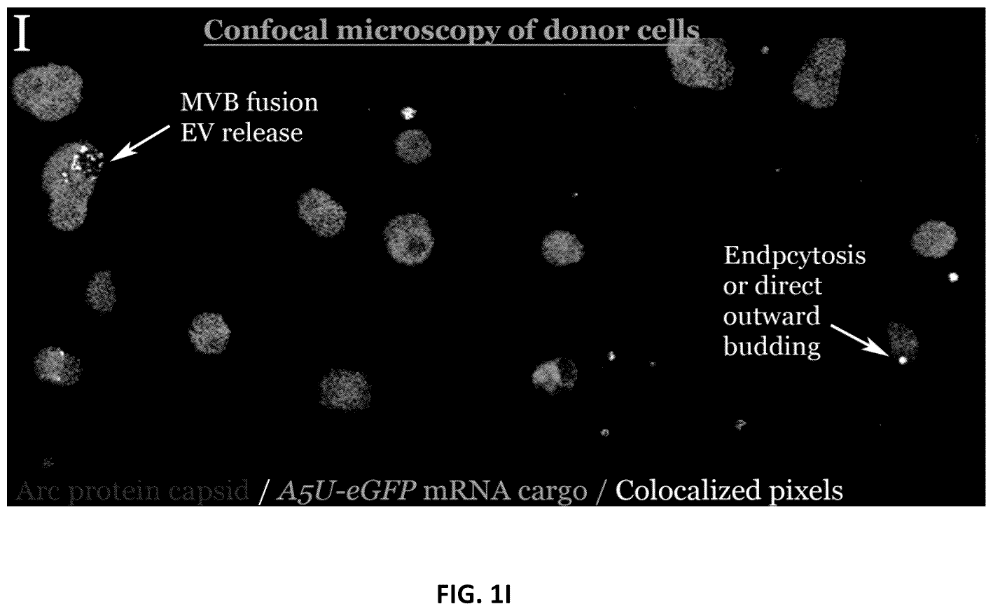

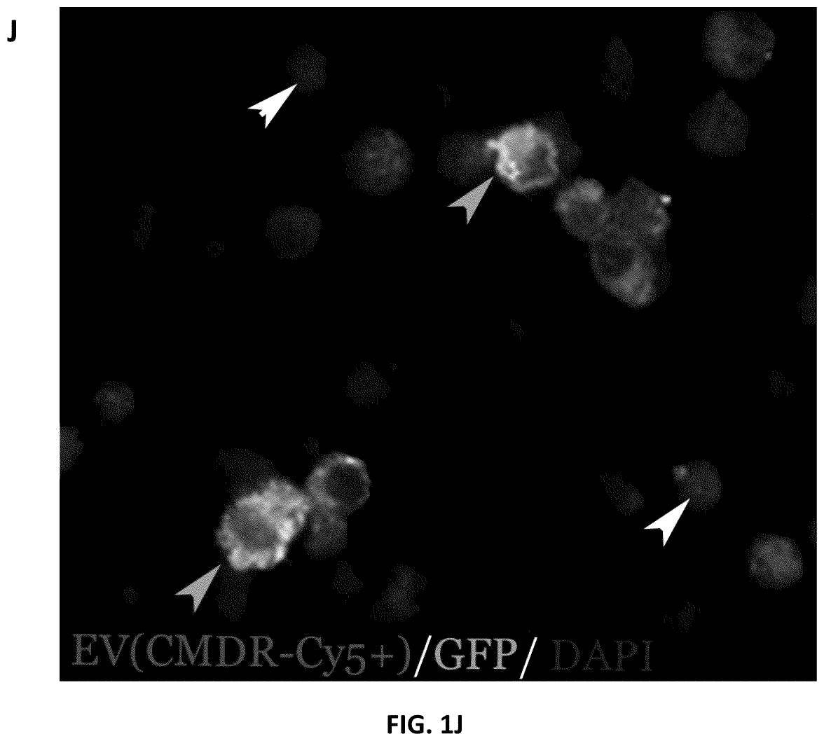

Arc vesicles were engineered, produced, isolated, and characterized, to verify their ability to deliver mRNA in vitro (FIG. 1A). Two components of this carrier system are the mRNA cargo and the Arc protein capsid, which can be introduced to virtually all donor cell types to produce enveloped eaEV with different homing/targeting capacity for various applications. The cargo construct was engineered for efficient mRNA encapsulation where the A5U sequence was added upstream of the cargo mRNA sequence (FIG. 1A).

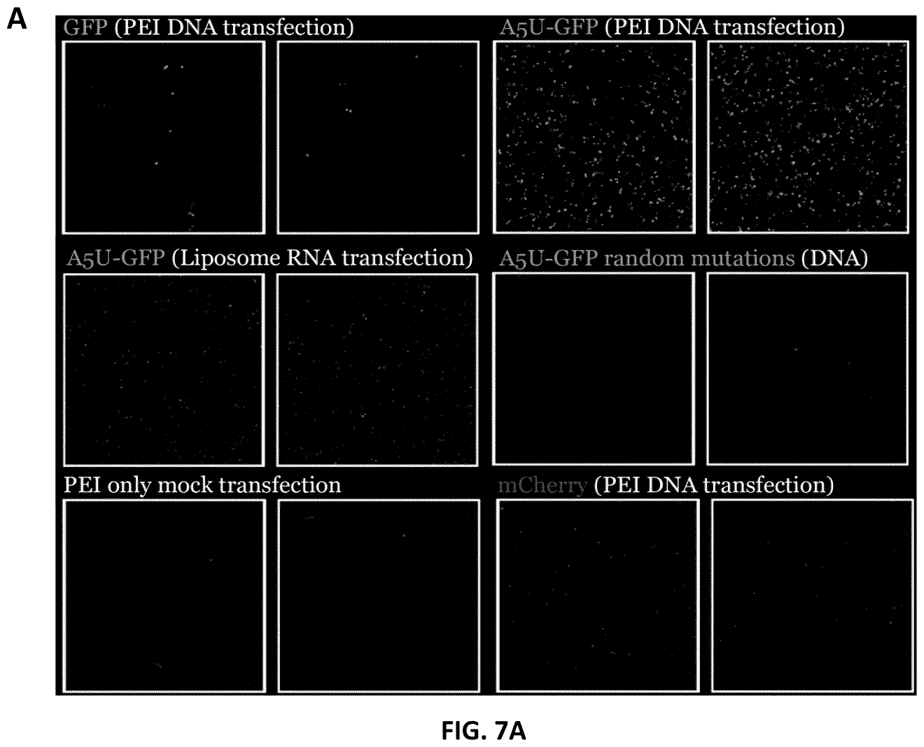

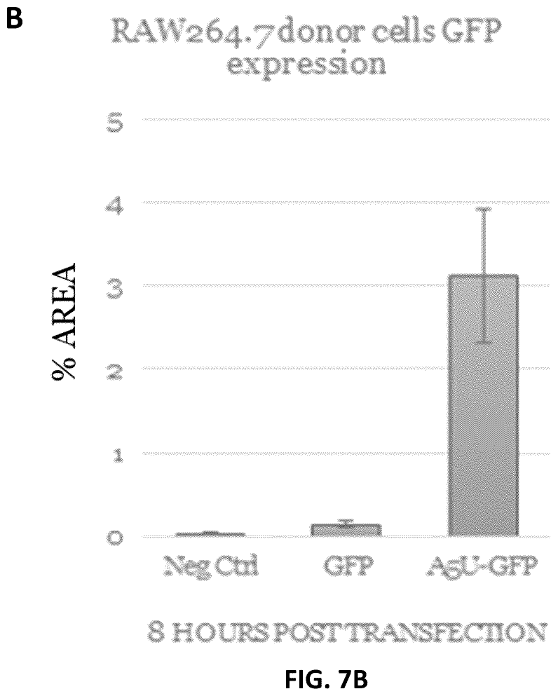

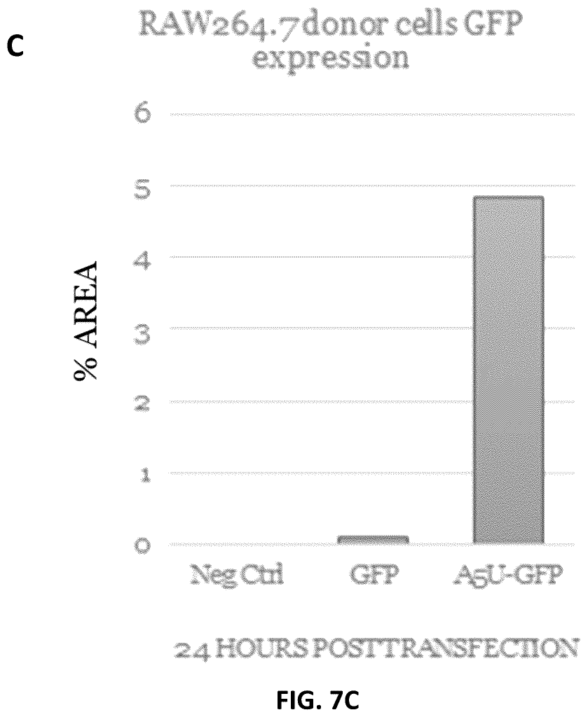

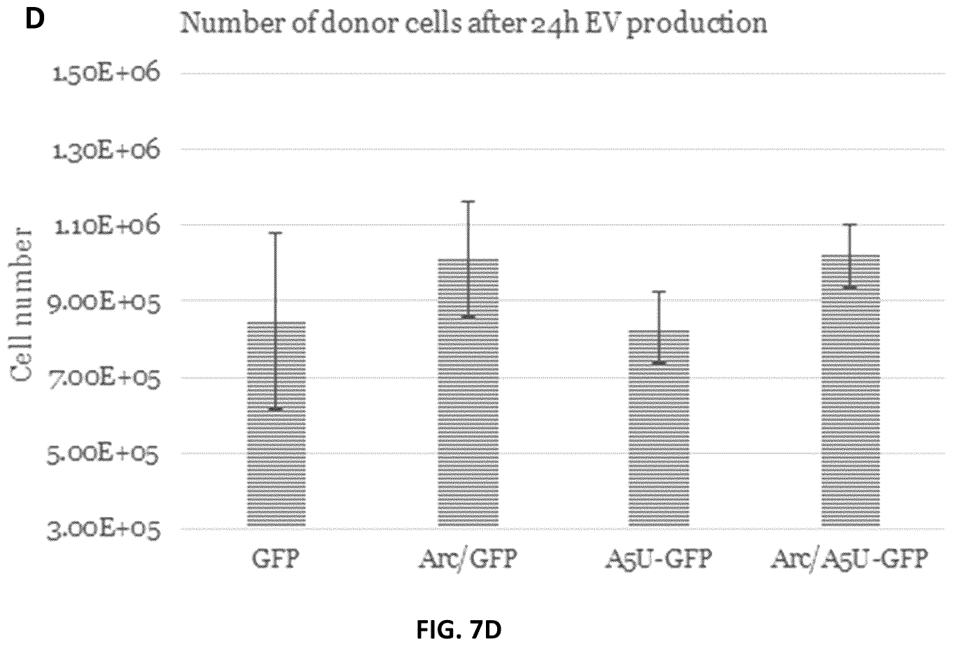

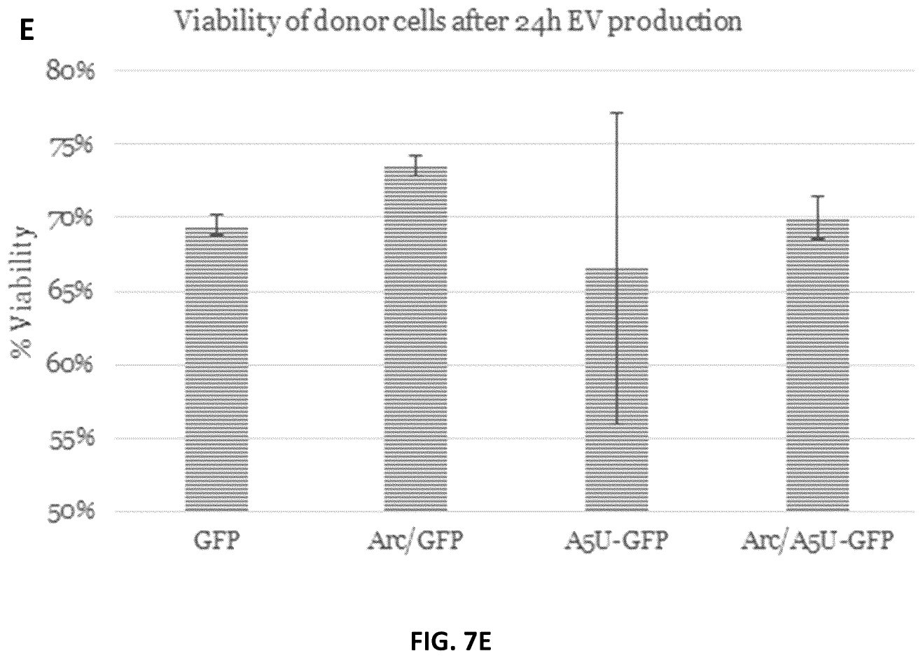

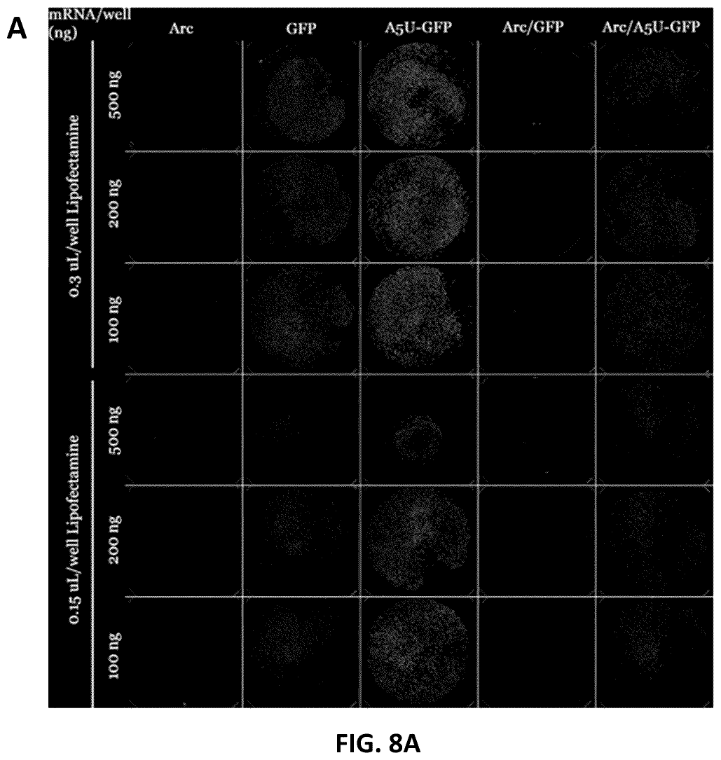

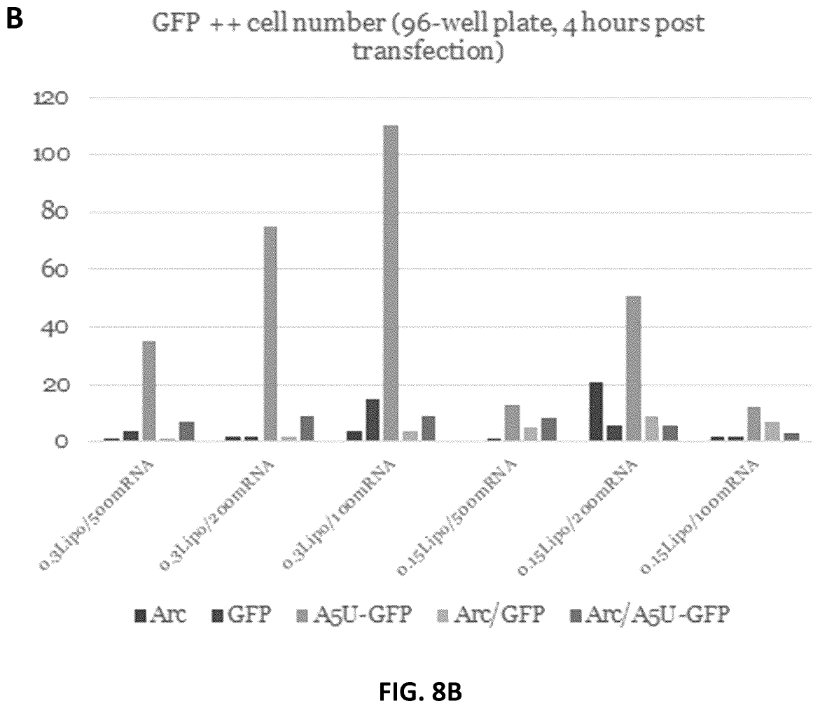

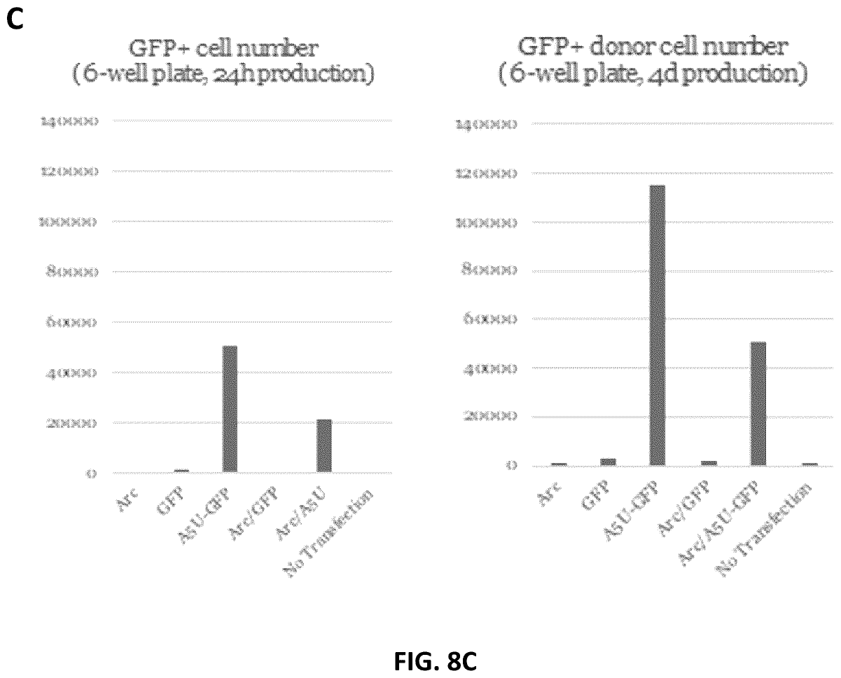

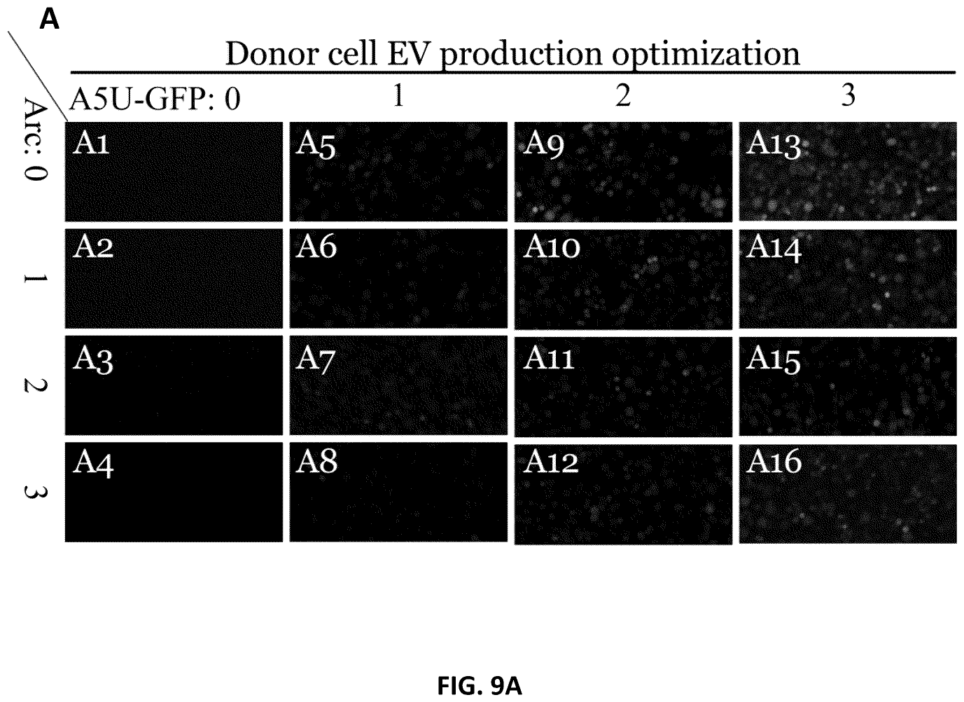

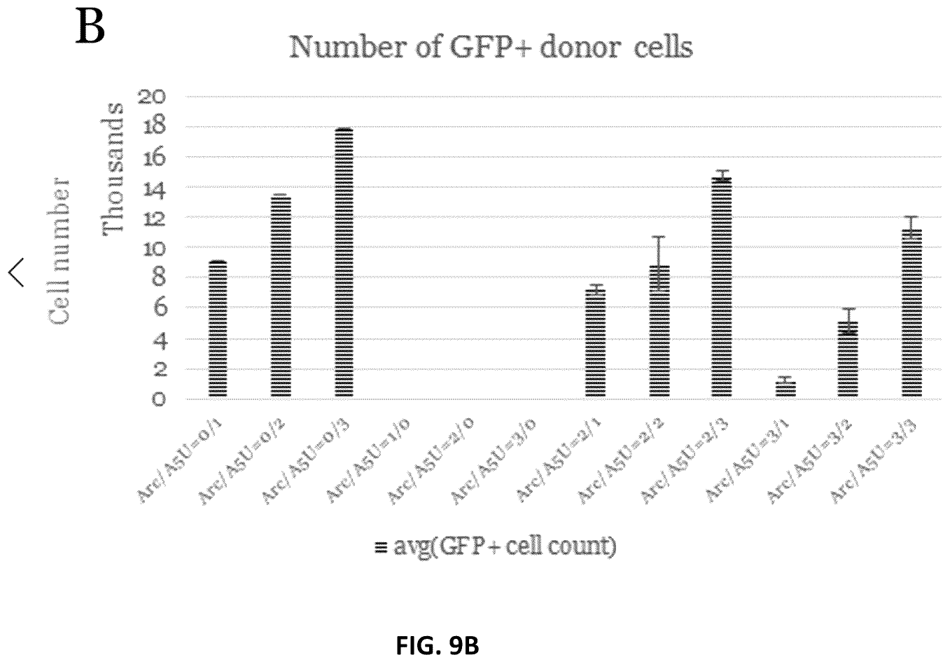

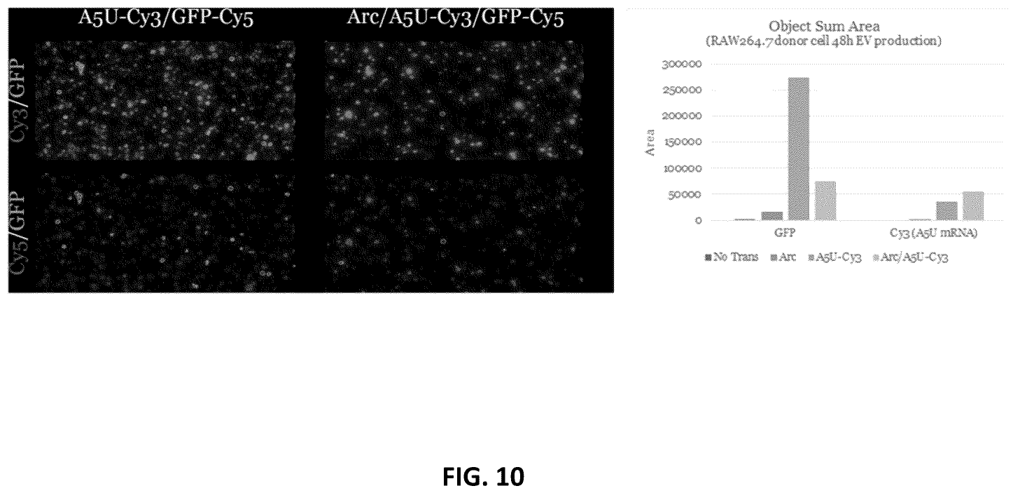

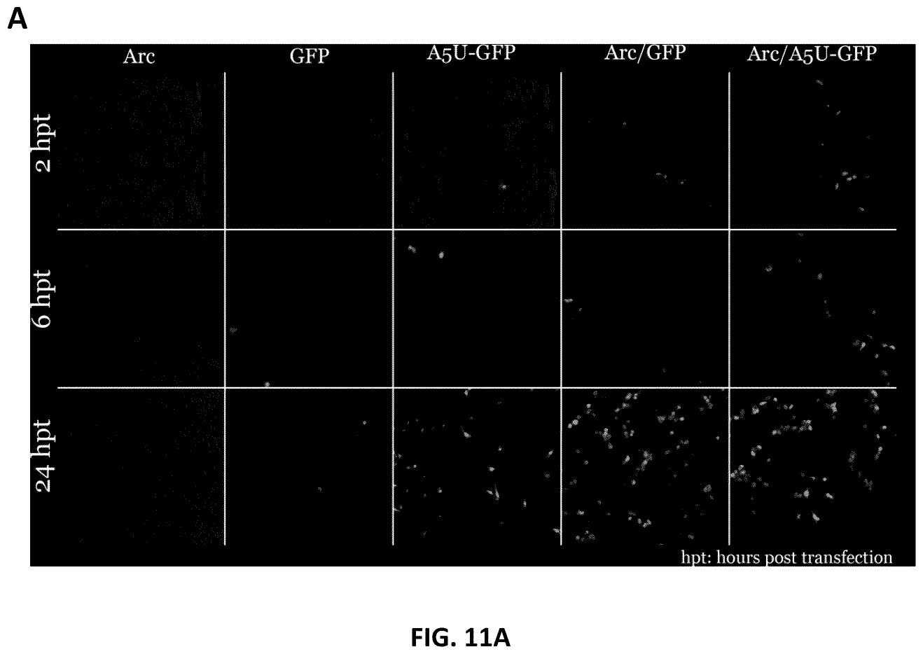

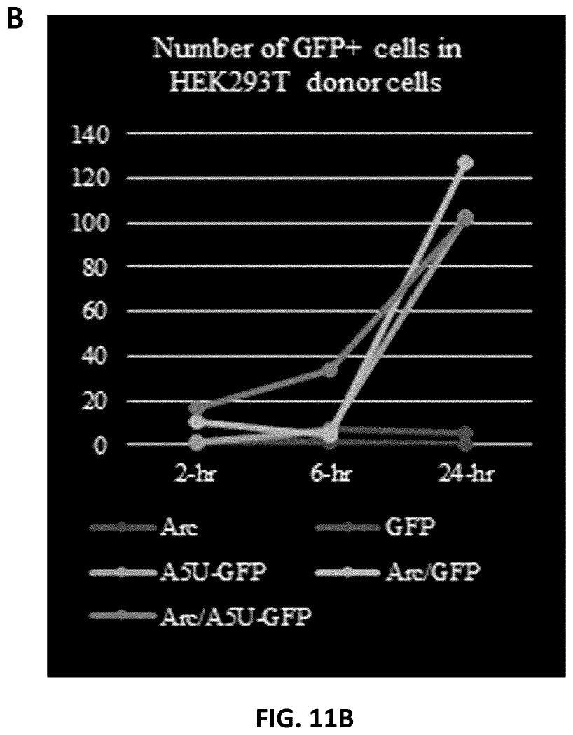

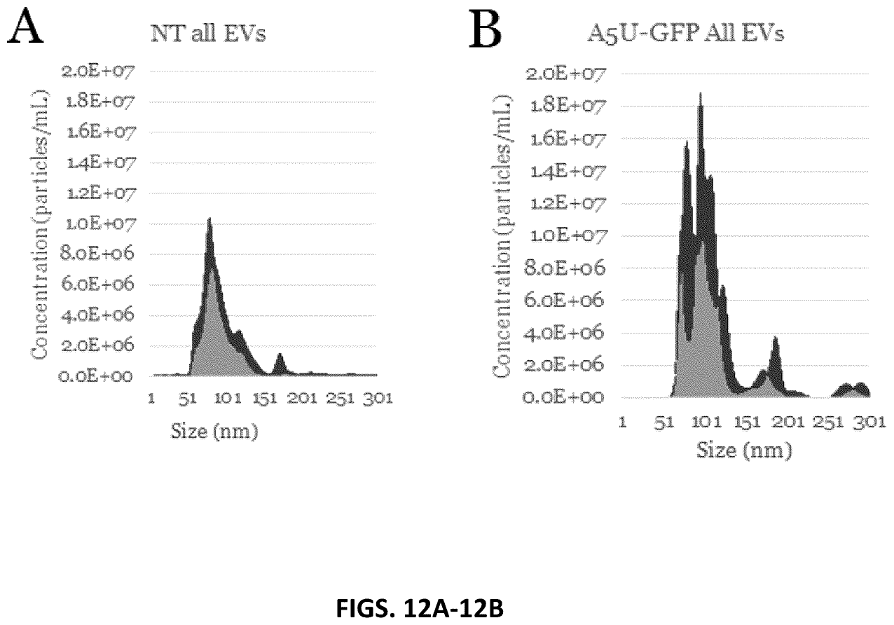

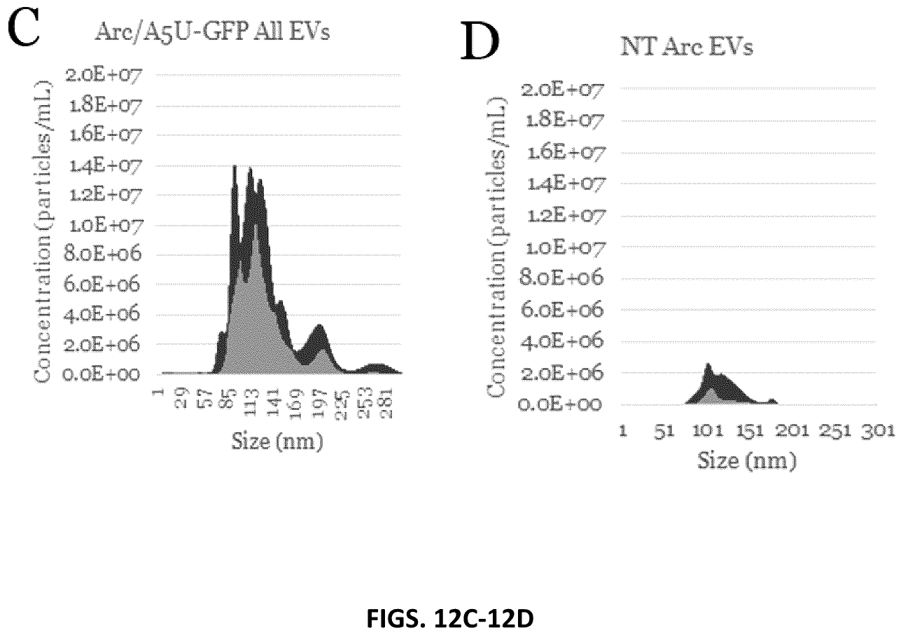

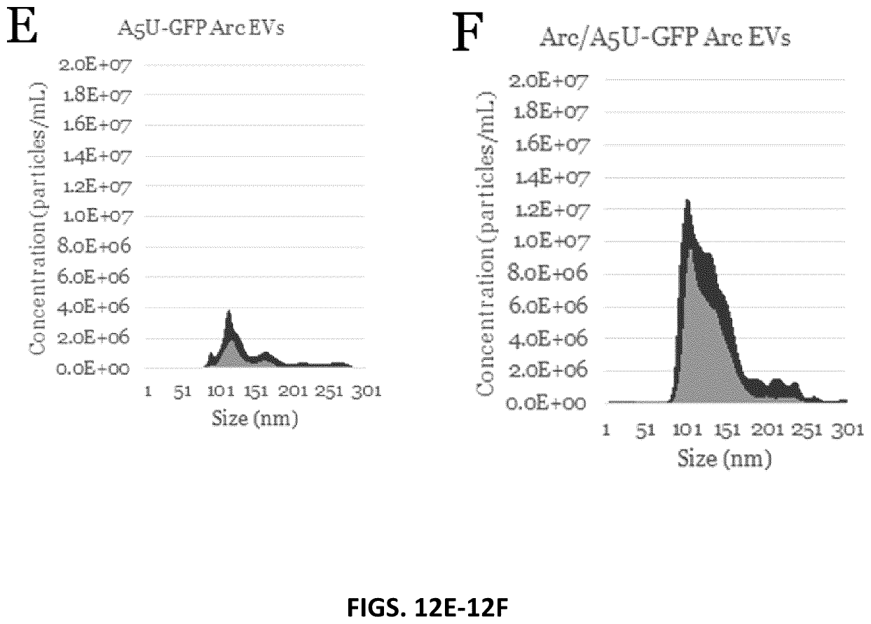

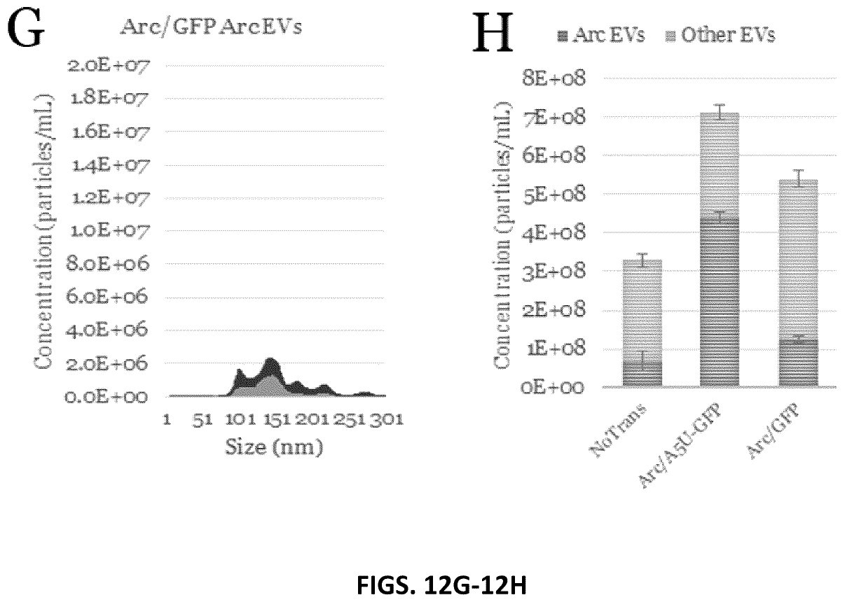

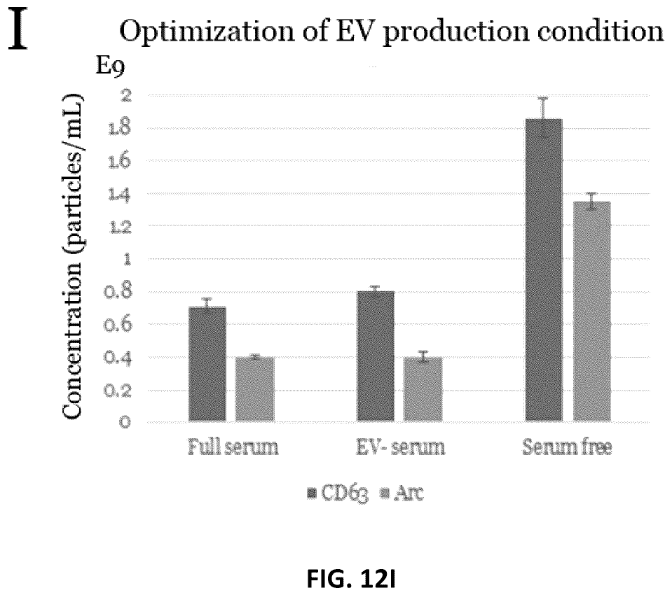

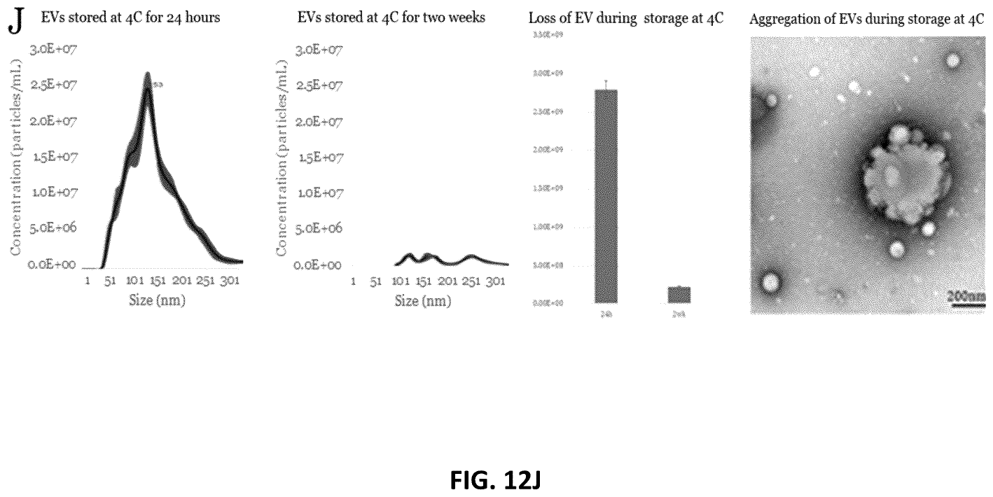

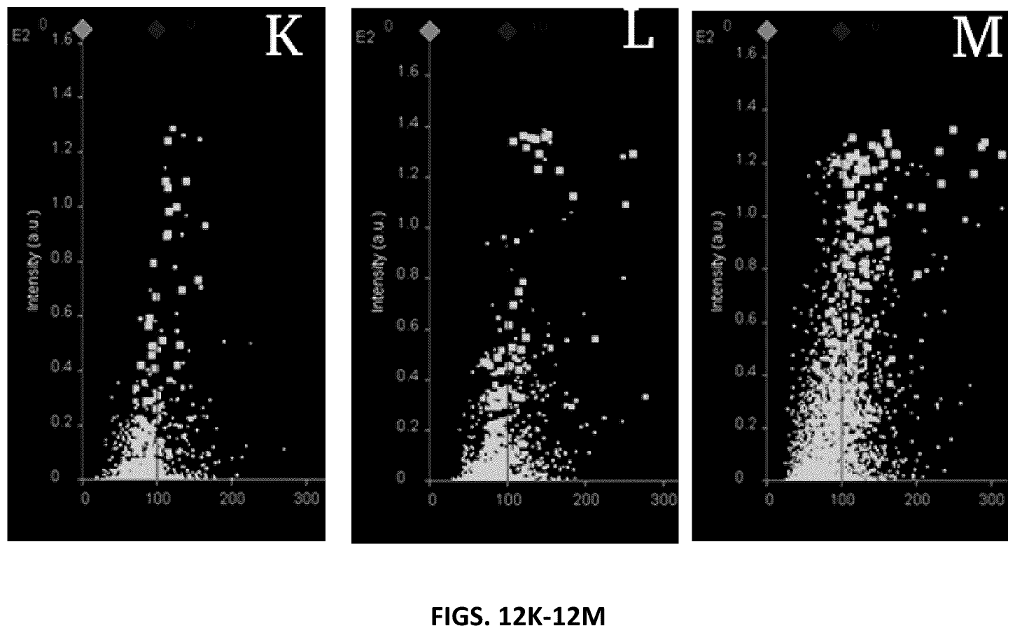

Rat Arc capsid was used for characterization since it can be distinguished from endogenous Arc in human/mouse cell lines as well as in vivo mouse models. To produce the engineered Arc EV (eaEV), mRNA encoding the Arc capsid and cargo GFP mRNA was delivered into human embryonic kidney (HEK293) and mouse macrophage (RAW264.7) cells (FIG. 1A). Extensive and comprehensive titration and time lapse experiments were carried out to optimize EV production (FIG. 7-FIG. 11). A variety of transfection methods were tested, including electroporation of DNA/RNA, PEI-DNA transfection (FIG. 7 & FIG. 10), and liposome-RNA transfection (FIG. 8-FIG. 10). The doses of transfection reagents were carefully titrated to ensure uncompromised donor cell viability post transfection (FIG. 7, FIG. 8), the ratio of capsid/cargo constructs to maximize mRNA encapsulation without introducing excessive capsid mRNA (FIG. 9), the time lapse of mRNA loading and cargo expression in donor/recipient cells to maximize EV collection efficiency (FIG. 11). Finally, we used liposome mediated RNA transfection was used to deliver 12 pmol Arc (4.63 μg), 8 pmol GFP (1.86 μg) and 8 pmol A5U-GFP (2.26 μg) mRNAs per one million donor cells, to produce control EVs and eaEVs for 8-40 hours in serum free culture medium. Isolated from the supernatant culture medium by ultrafiltration, the eaEV subpopulation was labeled by a fluorescent Arc antibody to be characterized via fluorescent nanoparticle tracking analysis (NTA), whereas total EVs were measured by light scattering from all particles (FIG. 1B-C and FIG. 12A-C). A general EV marker antibody (anti-CD63) and plasma membrane stains (CellMask) were also used to label total EVs. The Arc+ EV subpopulation appeared larger than Arc− vesicles (FIG. 1D and FIG. 12A′-C′). The addition of rat Arc mRNA increased Arc+ EVs by 6.5 fold, suggesting an efficient production of eaEVs (FIG. 1E-F).

Next, eaEV and the Arc capsid were examined by negative stain electron microscopy (NSEM, FIG. 1G), to further characterize their size and morphology. The fluorescence intensity of labeled eaEVs and total EVs was measured and corelated to the NTA results with coefficients calculated (FIG. 1H and Table S1). With these, a measurement of fluorescence intensity was used to calculate the absolute particle concentration of eaEVs among total EVs, using a fluorescent intensity reader. This confirmed the morphology, size distribution, production efficiency, and sufficient purity of collected eaEVs.

To visualize the secretion and transfer of individual eaEV, the Arc capsid protein and cargo mRNA were labelled by immunocytochemistry (ICC) and fluorescence in situ hybridization (FISH) with quantitative hybridization chain reaction (qHCR) followed by confocal laser scanning microscopy (CLSM) (FIG. 11). In addition to extracellular Arc+/GFP+ eaEVs, their outward budding (also possibly endocytosis) was often observed, whereas the release of cargo containing Arc+ EVs from the multivesicular body (MVB) was a rare event (FIG. 1I). EVs have been classified into ectosomes and exosomes based on their size and biogenesis differences. Like its homologs, viral capsid gag proteins, Arc appeared to mediate direct outward budding of ectosomes, which are larger than exosomes that are released from the MVB fused to donor cell membrane. Indeed, NTA size distribution confirmed donor cell culture imaging that eaEVs are more likely ectosomes than exosomes (FIG. 1D and FIG. 12A′-12C′). Upon transduction into recipient cells, successful eaEV uptake and cargo translation in recipient RAW264.7 cells were shown by live-cell epifluorescence microscopy (EFM) of an EV membrane stain and the GFP expression (FIG. 1J). GFP was observed only in recipient cells with CMDR (CellMask Deep Red), a plasma membrane stain used to label EVs after their isolation from the donor cell culture before their transfer into recipient cells. Altogether this data shows engineered, produced, and isolated eaEV that can load and deliver mRNA.

### Example 2. A5U-eaEV can Load mRNA with Improved Efficacy and Selectivity

Selective packaging of the 5′UTR of the HIV-1 genome depends upon Gag protein intact capsid (CA) domain lattice. Therefore, Arc protein may bind to the A5U through ionic interactions in its N-terminus.

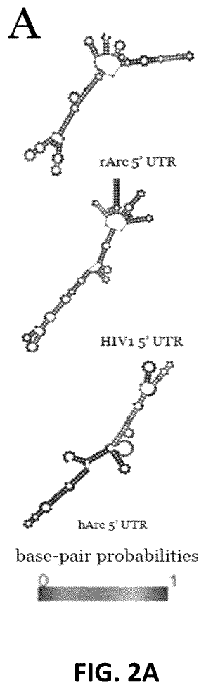

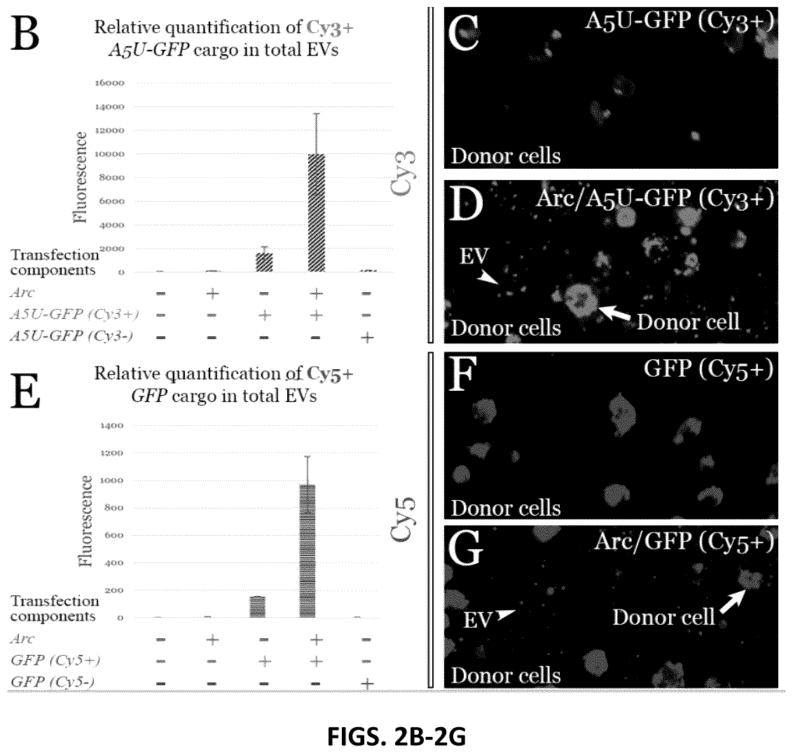

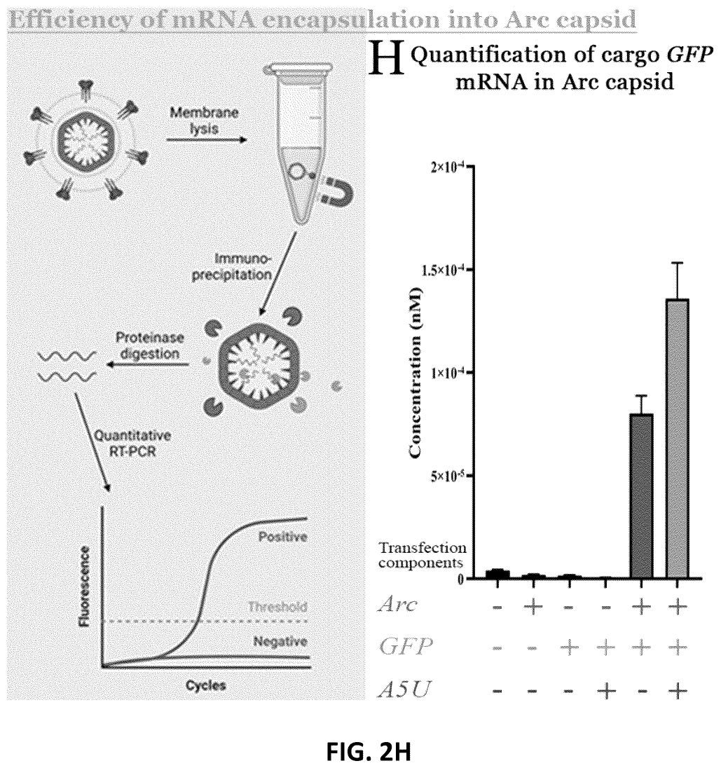

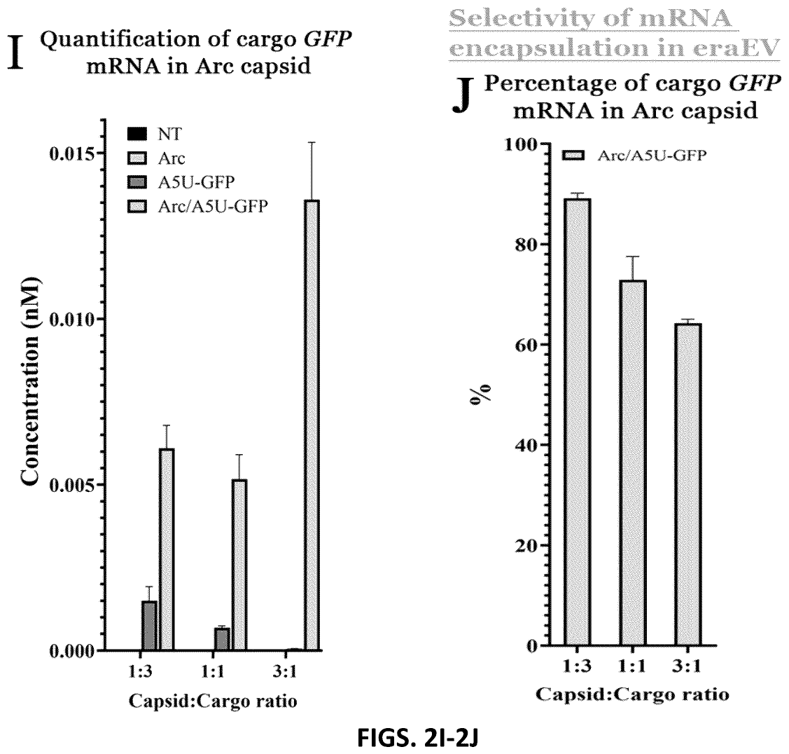

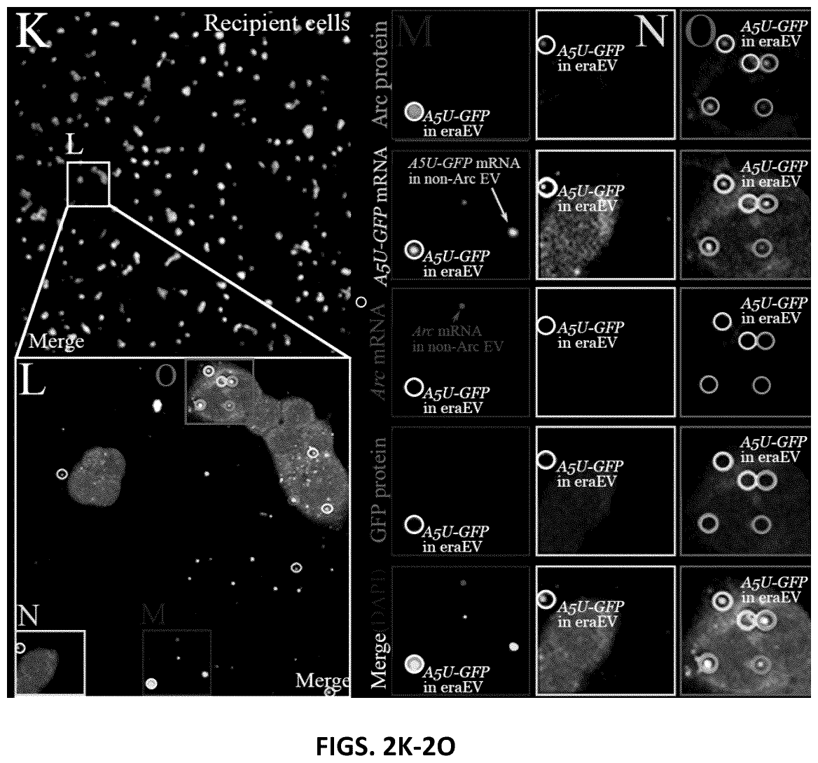

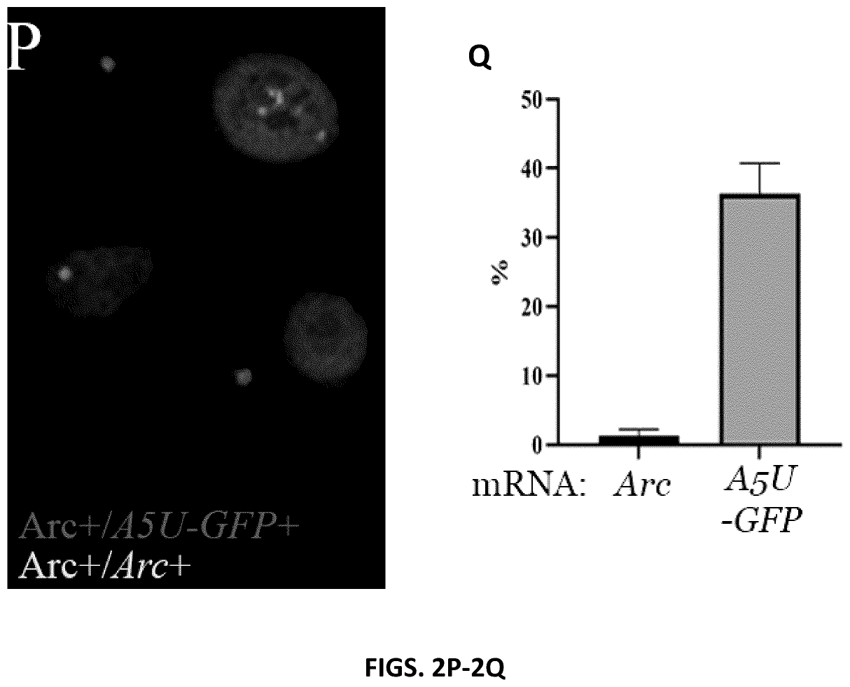

As such, it was hypothesized that adding the A5U could significantly improve mRNA cargo loading efficiency. As such, to enable efficient cargo loading, the rat A5U was added to the cargo construct, as it shows more similarity in predicted secondary structure as the 5′UTR of HIV1 RNA genome, compared to those of other species (FIG. 2A). Cargo constructs with and without the 5′ UTR, A5U-GFP and GFP mRNA transcripts were transcribed in vitro (NEB, CleanCap, T7-AG, with pseudoUTP) using fluorescently labelled Cy3- and Cy5-UTP, respectively. EVs were produced by transfecting combinations of these mRNAs into the donor cell, of six control and experimental groups: (1) mock transfection (NT); (2) Arc; (3) GFP; (4) A5U-GFP; (5) Arc/GFP; (6) Arc/A5U-GFP. Fluorescence intensity reading of isolated control and engineered EVs carrying Cy3+ A5U-GFP and Cy5+ GFP cargo was performed to compare cargo loading efficiency of these EVs. The results showed that Arc significantly promoted mRNA loading into EVs (FIG. 2B&E). Confirming these results, epifluorescence microscopy of donor cell culture showed drastically increased amount of extracellular Cy3+/Cy5+ cargo mRNAs (FIG. 2C-D&F-G).

To further characterize the cargo inside the Arc capsid, RNA immuno-precipitation (RIP) followed by RT-qPCR was performed. After the lysis of EV outer membrane, an Arc antibody was used to immuno-precipitate the Arc protein capsid, which was subsequently digested to release its mRNA cargo for the quantification by RT-qPCR. These experiments showed that the A5U increased cargo mRNA encapsulation (FIG. 2H). With an optimized ratio between the capsid and cargo constructs transfected into the donor cell, the engineered Arc EV enabled both efficient (FIG. 2I) and selective (FIG. 2J) loading of A5U-GFP, among other abundant RNAs, including rArc, GAPDH, a cytosolic housekeeping gene, and 18S, one of the most prominent RNA species found in ectosomes.

Despite an overexpression of capsid Arc mRNA in the donor cell, the cargo A5U-GFP seemed favorably enriched into the capsid than Arc itself, as long as not an excessive amount of Arc mRNA was transfected to compete for encapsulation. When the ratio of (1× Arc:3× A5U-GFP) was used, Arc mRNA was merely observed in any extracellular eaEV (FIG. 2K-Q). Recipient HEK293 cells with A5U-eaEVs (Arc:A5U-GFP=1:3) were studied by immunocytochemistry (ICH) and fluorescence in situ hybridization (FISH) with quantitative hybridization chain reaction (qHCR) followed by confocal laser scanning microscopy (CLSM). The capsid protein, the cargo mRNA, and the capsid mRNA in each individual EV was visualized at extremely high resolution and their overlap was quantified in ImageJ, using a custom ImageJ macro code. This revealed that eaEV prefer to load a cargo RNA with the A51 in comparison to the capsid Arc mRNA without the A5U. On the other hand, while introducing a ratio of (3× Arc: 1× A5U-GFP), a significant amount of Arc and 18S would be encapsulated in the Arc capsid (FIG. 2J). Further optimizing the ratio between capsid and cargo RNA transfection components, it was found that with the same amount of cargo mRNA present, GFP expression in the donor cells decreased when more capsid mRNA was added (FIG. 9). This is because more capsids were synthesized to encapsulate the cargo mRNA decreasing the amount of free mRNA left for translation. Considering all these factors, it was decided to transfect a ratio of [1.5× Arc (12 pmol): 1× A5U-GFP (8 μmol) per 1 million cells] to achieve efficient and selective cargo loading without introducing excessive Arc mRNAs.

Arc plays critical roles in the CNS and its overexpression in the drug delivery system should be avoided. Because of this, the ratio between transfection components was optimized via thorough characterization powered by high-resolution qHCR and extremely sensitive RIP-qPCR, both of which enable single EV analysis with accurate quantification. The Arc EV were further engineered by adding an mRNA motif, A51, enabling highly efficient and selective packaging of the mRNA cargo.

### Example 3. A5U-eaEV can Improve the Delivery Efficacy and the Stability of mRNA Cargo

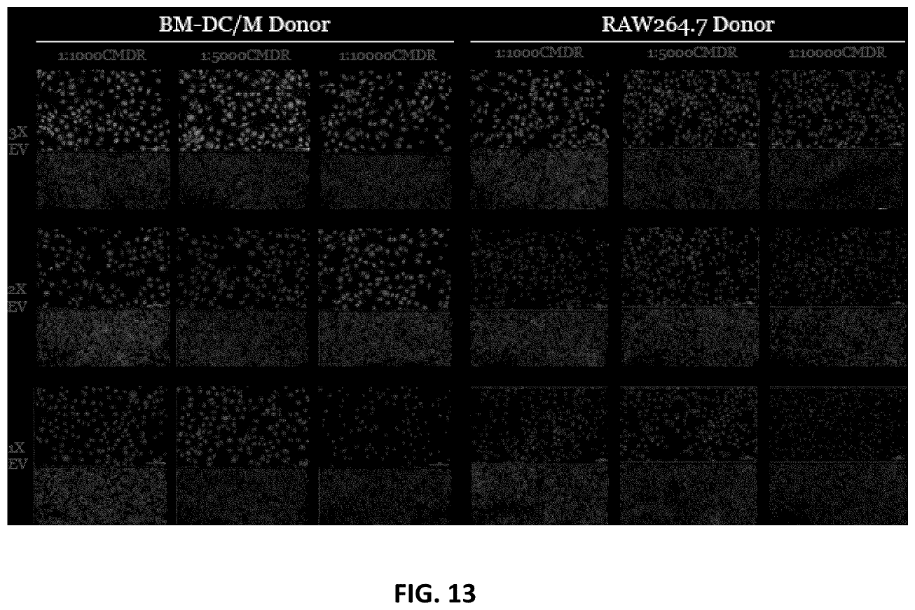

The effectiveness and stability of A5U-eaEV as an mRNA drug carrier was verified by stably increased cargo mRNA uptake in recipient cells for over one week period (FIG. 3A1). Interestingly, without the A51), RNA transduction into recipient cells seemed less efficient and, more importantly, less stable (FIG. 3A2). The mechanism is likely similar to HIV gag which requires the 5′UTR of its own genome to stabilize the capsid. The behavior of EVs after their transfer onto recipient cells is shown in FIG. 3B1-D6: at 15 minutes EVs carrying fluorescent mRNA cargo started docking onto the recipient cell membrane (FIG. 3B1-B4); from 1 hour onwards intracellular fluorescence was observed and by 4 hours virtually all recipient cells received Cy3+ A5U-eaEVs while controls were less efficient (FIG. 3C1-C4); this advantage in uptake efficiency was greatly expanded over time with not only had all cells the cargo but also more cargo in each cell (FIG. 3A1 & D1-D6). It is necessary to emphasize that the same amount of total EVs was added to each sample group. The rapid fluorescence reading to quantify total EVs using the CMDR dye was used as was done in Example 1 above. With careful optimization of the dye concentration, this method accurately quantified the relative amount of total EVs (FIG. 3E and FIG. 13). CMDR+ EVs was added to recipient cells, measured the mean fluorescence 1 hour later and the result suggested that similar numbers of total EVs were taken up by recipient cells (FIG. 3F). Thus, A5U-eaEV significantly improved the delivery efficiency and stability of mRNA cargo.

A consistent increase in GFP expression via A5U-eaEV delivery (FIG. 3G) was also observed. Despite Arc/A5U-GFP substantially promoting mRNA encapsulation in donor cells and mRNA uptake by recipient cells, RAW264.7 recipient cells showed weak and sparse GFP expression. This is likely because eaEV cargo release and translation are activity dependent in not only neurons but also immune cells. In addition to RAW264.7, this increase in cargo translation has also been verified in triple negative breast cancer cells (MDA-MB-231, FIG. 3H). In conclusion, A5U-eaEV can deliver mRNA with improved efficiency and stability in vitro.

### Example 4. Leukocyte Derived A5U-eaEV can Efficiently Deliver mRNA Across the BBB Specifically Targeting Neuroinflammation

The blood brain barrier (BBB) is a highly dynamic and selective semipermeable border separating the peripheral circulation from the central nervous system (CNS), preventing the entry of large molecule pharmaceuticals into the brain. The BBB is composed of continuous brain microvascular endothelial cells (BMEC), their tight junctions, basement membranes, pericytes, and astrocyte terminals. BMECs normally express low levels of leukocyte adhesion molecules compared to peripheral endothelial cells to prevent the margination and transmigration of immune cells into the brain. The BBB is disrupted by aged-associated low-grade inflammation. also termed inflammaging, neurodegenerative disease, as well as more severe pathological changes such as systemic inflammation and secondary injury (e.g., stroke). In response to these inflammatory stimuli from the brain, BMECs have been shown to exhibit increased permeability and elevated expression of leukocyte adhesion molecules, allowing more leukocytes (e.g., MΦs and DCs) and leukocyte derived EVs to enter the brain across the BBB. Although leukocyte EVs can enter the brain independently without involving brain infiltrating immune cells, the accumulation of such EVs becomes increased in the inflamed brain with more permeable BBB. This makes the EVs great candidates for brain drug delivery.

In addition to the neuroinflammation targeting capacity of leukocyte EVs, the native role of Arc EVs in inter-neuronal mRNA transfer should further improve the neuronal uptake of these vesicles. Moreover, the Arc capsid protects cargo mRNA from RNase degradation until the release is triggered, increasing their stability. Given these native advantages of leukocyte eaEV and the observed improvements increasing mRNA cargo loading above, these EVs may sufficiently deliver mRNA into the CNS targeting neuroinflammation.

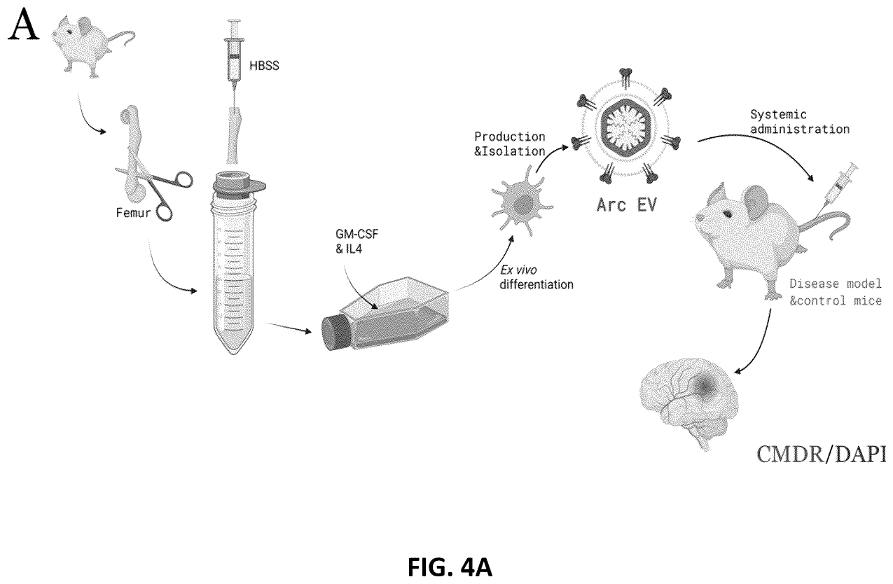

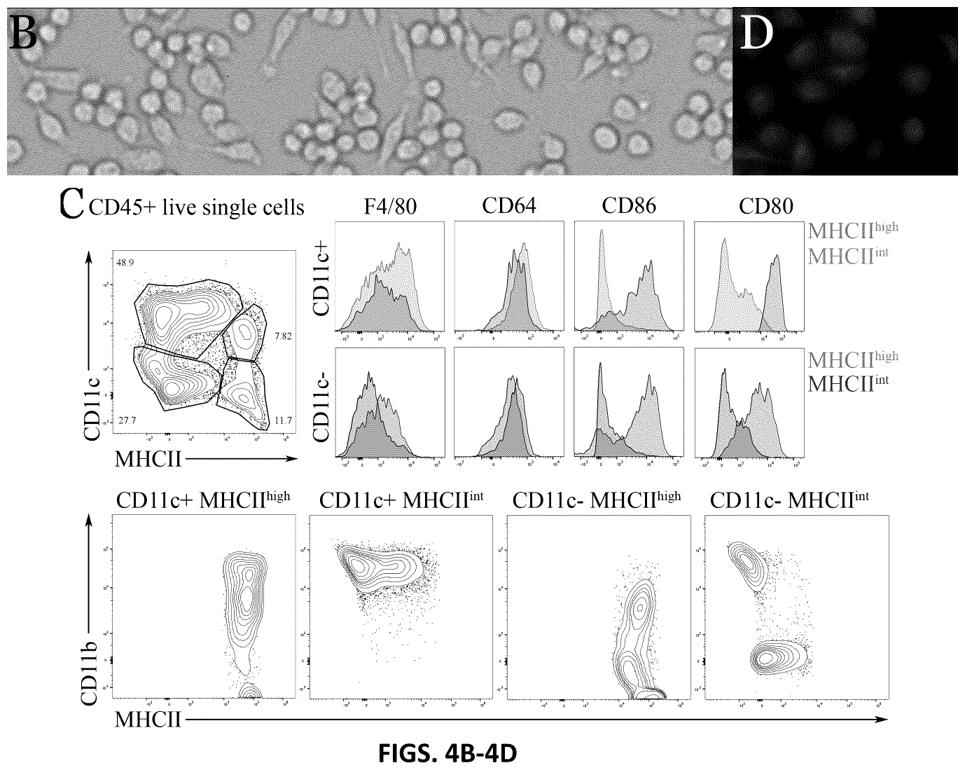

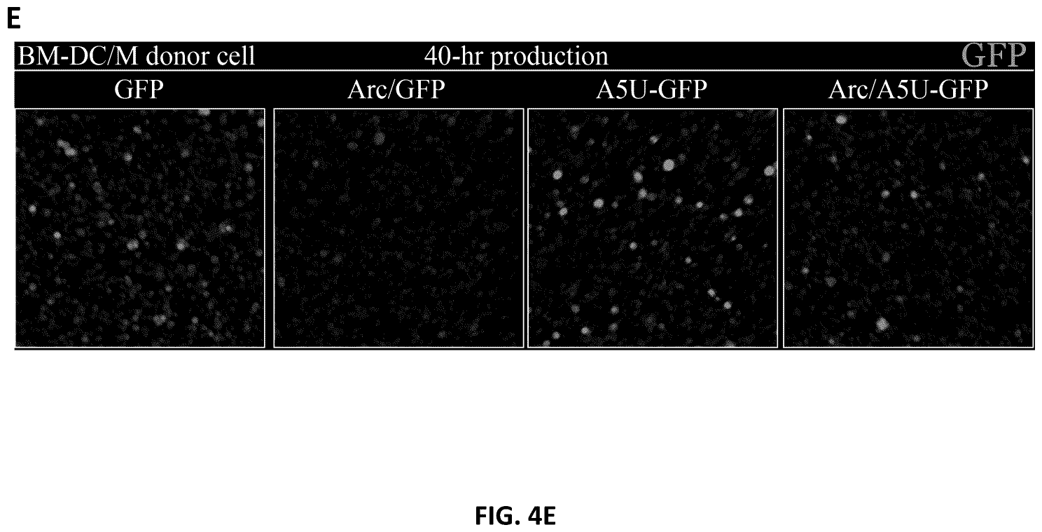

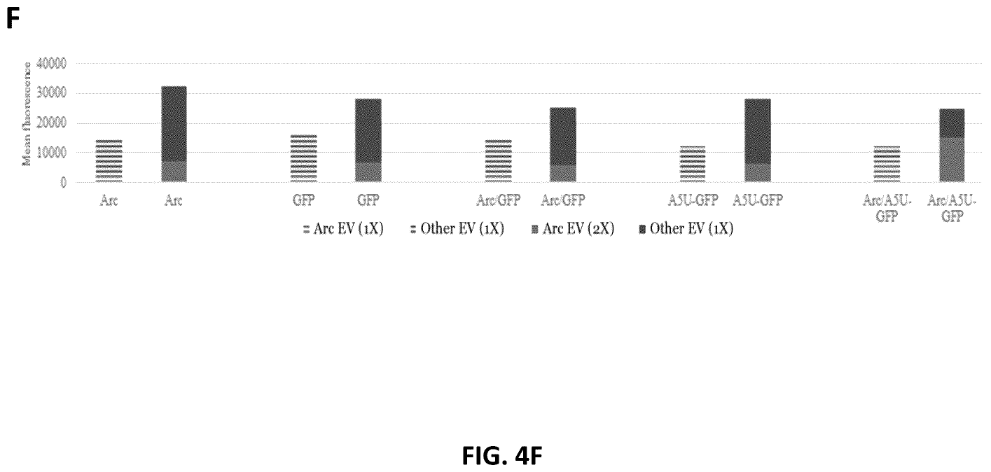

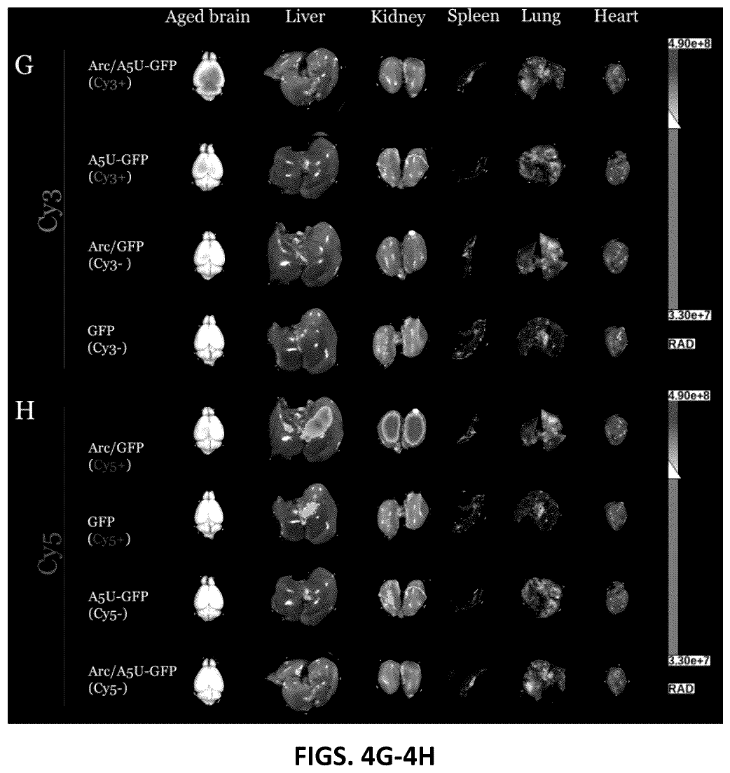

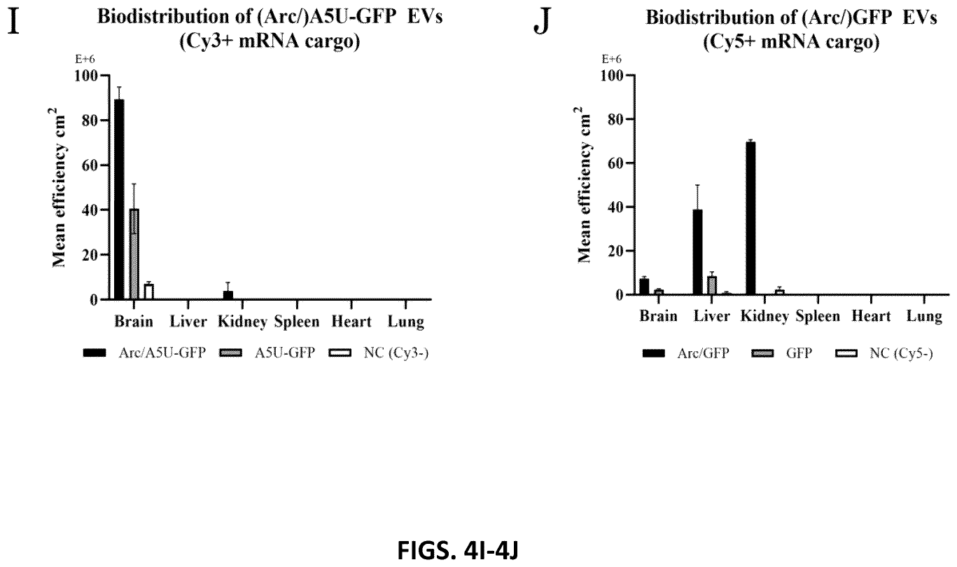

To produce a pool of immunologically inert EVs for in vivo studies, self-derived donor leukocytes were differentiated ex vivo from BM cells harvested from mice with a homogenous major histocompatibility complex (MHC) haplotype. Upon isolation from the femur, BM cells were cultured with GM-CSF and IL-4 for 7 days (FIG. 4A-B). EVs from both MΦs and DCs have been demonstrated to cross the BBB, therefore a culture protocol for both populations was utilized, culturing subpopulations including but not limited to monocyte-derived DC, monocyte-derived MΦ, and conventional DC (FIG. 4C).

As described above, control and experiment groups of EVs were produced, isolated, and characterized by transfecting: (1) mock transfection negative control (NC); (2) Arc; (3) GFP; (4), A5U-GFP; (5) Arc GFP; (6) Arc A5U-GFP. BM-DC/MΩ can uptake its own EVs (FIG. 4D) Fine tuning the balance between secretion and uptake was critical. For these experiments, depending on the donor cell confluency, EVs were produced for 24-48 hours to reach a saturation of EVs in the supernatant medium, with total EV concentrations monitored and measured via CMDR epifluorescence intensity in time lapse experiments. Before collection, each control and experimental group were verified to contain about the same amount of total EVs. Despite differences in the proportion of eaEVs: the Arc/A5U-GFP group showed the highest proportion of eaEVs among total EVs (FIG. 4F). This difference may be due to either a higher production or stability of A5U-eaEV, as this measurement was done 42-hour post transfection (40 hours production+2-hour purification/staining) leaving plenty of time for EV degradation. To explore the potential of leukocyte-eaEV in targeting highly inflammatory brain regions across the BBB, the same amount of total EVs per each gram body weight of mice were intravenously (IV) injected into various neuroinflammation models.

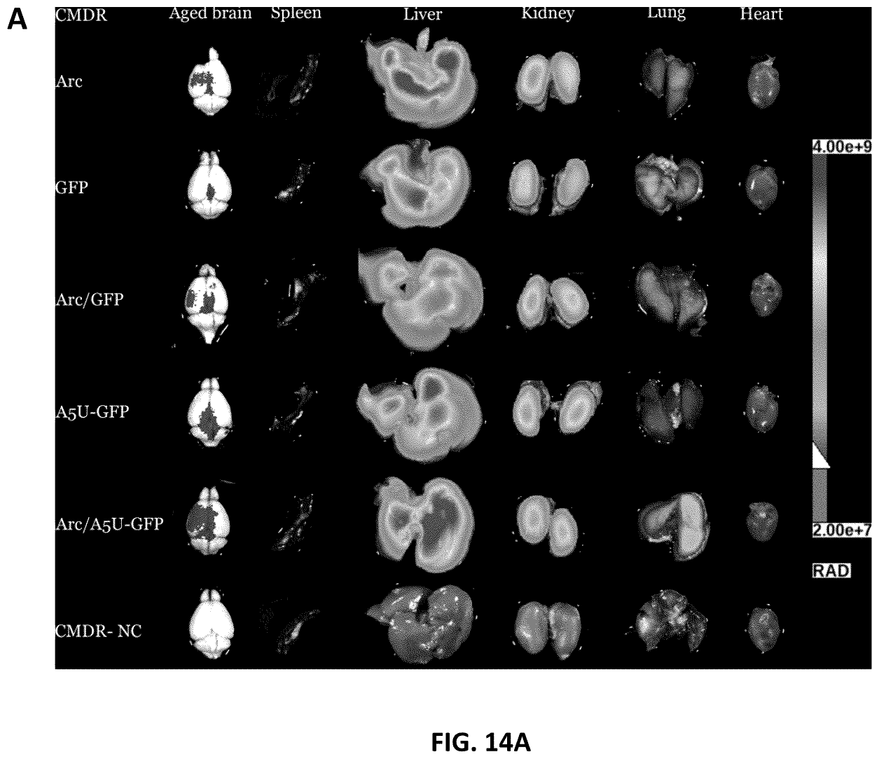

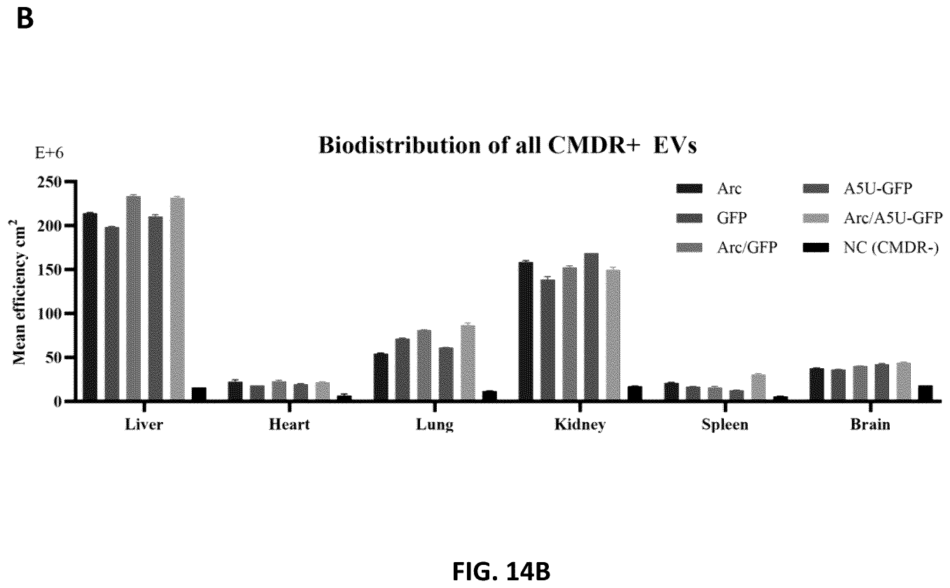

To study the in vivo biodistribution of eaEVs in a pan-neuronal inflammation model, leukocyte EVs (9E+07 total EVs per gram of body weight) were injected intravenously into aged (90-week weighting ˜40 g) mice with organs collected 72 hrs later following transcardial perfusion. Cy3+ and Cy5+ fluorescently labeled mRNAs enabled the visualization of eaEV biodistribution and cargo uptake via IVIS (in vivo imaging system). For the IVIS analysis of total EV biodistribution, non-fluorescent mRNAs were transfected and labeled total EVs collected with the CMDR plasma membrane stain. Various organs (brain, liver, spleen, kidney, heart, lung) of these aged mice were perfused, fixed, dissected, and imaged by IVIS. Among experimental and control groups, a similar level of the CMDR signal was observed, representing the biodistribution of total EVs or degraded EVs, in each organ upon collection (3-day post IV injection) (FIG. 14). However, Arc clearly increased mRNA delivery in vivo (FIG. 4G-H). Surprisingly, A5U further increased the accumulation of mRNAs in the aged brain (FIG. 4G&I), whereas the GFP mRNAs, also delivered by Arc EVs, ended up mostly in the liver and kidney (FIG. 4H&J). This increase in mRNA uptake into the aged brain is similar to the in vitro uptake results (FIG. 3B1-B2), potentially due to an increase in eaEV production or a higher stability. It is known that the formation of Arc capsid requires RNA. In mouse and human donor cells, the addition of rat A5U may further stabilize the rat Arc capsid, leading to a higher production efficiency while stabilizing eaEV at the same time. Therefore, BM-DC/MP derived A5U-eaEV can specifically deliver mRNA across the BBB targeting chronic pan-neuronal inflammation.

### Example 5. Leukocyte Derived A5U-eaEV can Efficiently Deliver mRNA Across the BBB for Pan-Neuronal Expression Under Chronic Inflammation

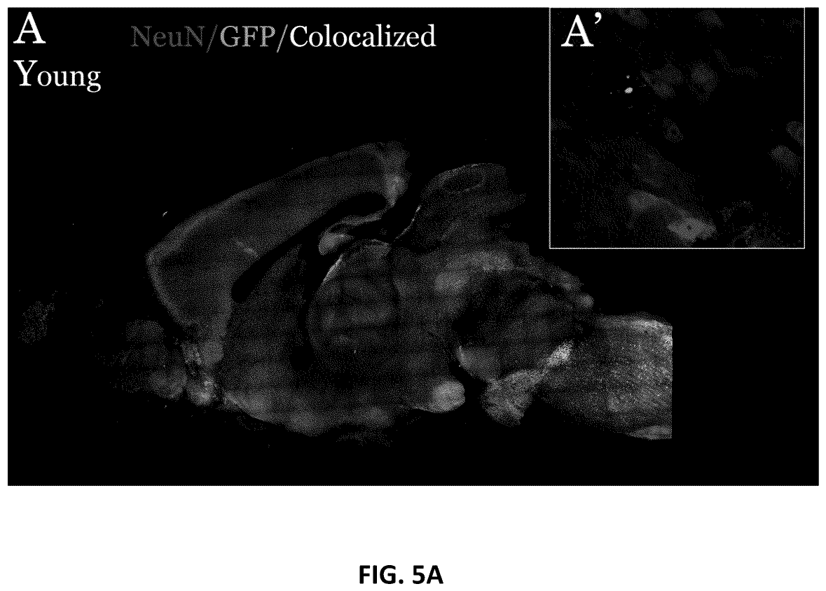

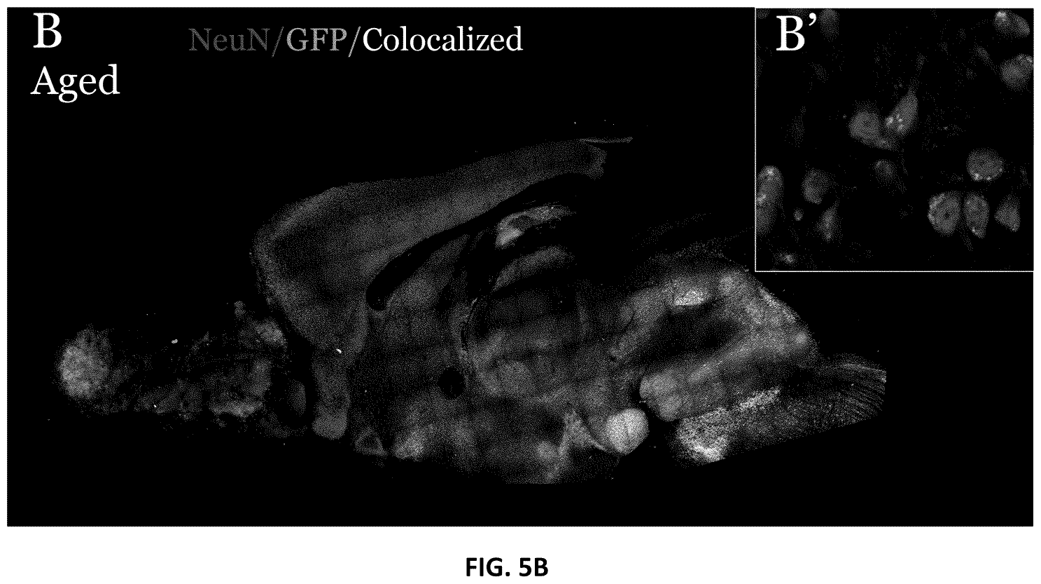

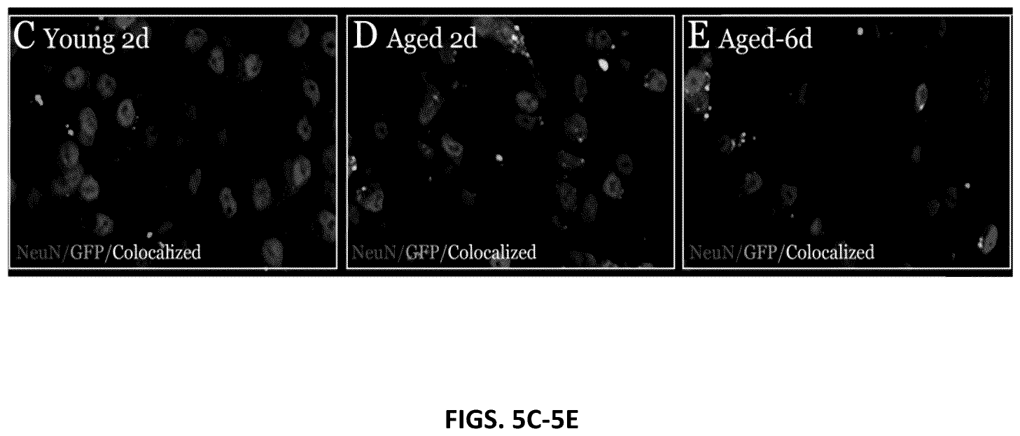

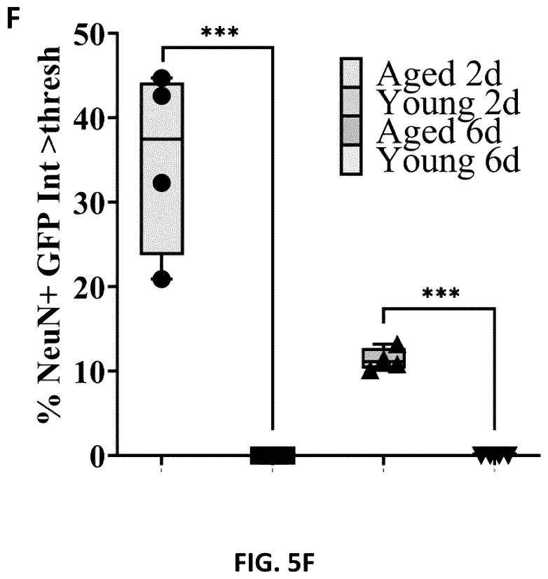

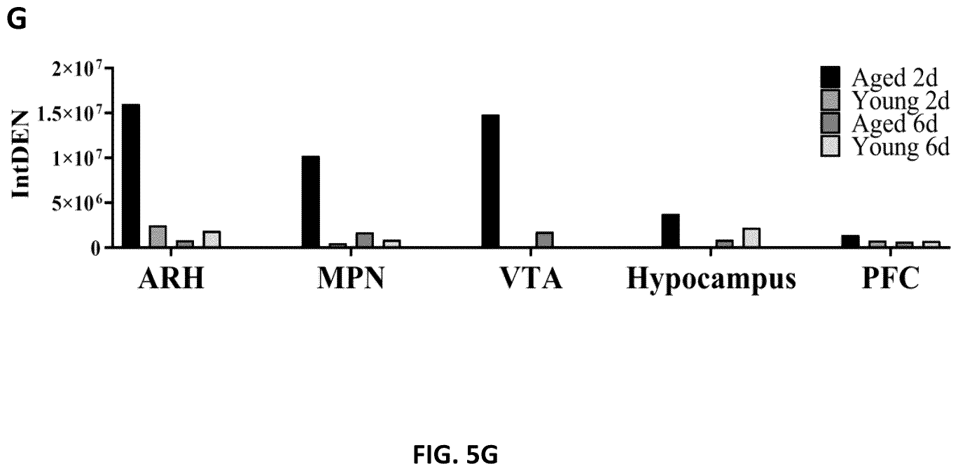

Protein translation from cargo mRNA was examined in the brain. A5U-eaEVs were administered systemically to deliver A5U-GFP mRNA into aged (˜90 weeks) and control young mice (<24 weeks), whose brains were collected at 2 and 6 days after the administration. Neurons were labeled by NeuN (Fox-3, Hexaribonucleotide Binding Protein-3) immunohistochemistry (IHC) staining. A higher level of neuronal GFP expression (NeuN+/GFP+) was observed in the aged brain compared to the young control (FIG. 5A-E). Interestingly, in young control brains, GFP was expressed comparably to the aged brain in blood cells inside the microvessels (FIG. 5A′inset of FIG. 5A, green) or in infiltrated immune cells (FIG. 5C, green). However, only aged brains showed significant neuronal expression (FIG. 5B′inset of FIG. 5B; FIG. 5D, white). Certain brain regions (e.g., hypothalamus, FIGS. 5C-D) absorbed and expressed more cargos than others (e.g., cerebral cortex). Altogether, this aging model demonstrated the pan-neuronal delivery of mRNA by eaEV responding to chronic inflammation across the whole brain.

### Example 6. Leukocyte Derived A5U-eaEV can Efficiently Deliver mRNA Across the BBB for Specific Local Expression Under Acute Injury

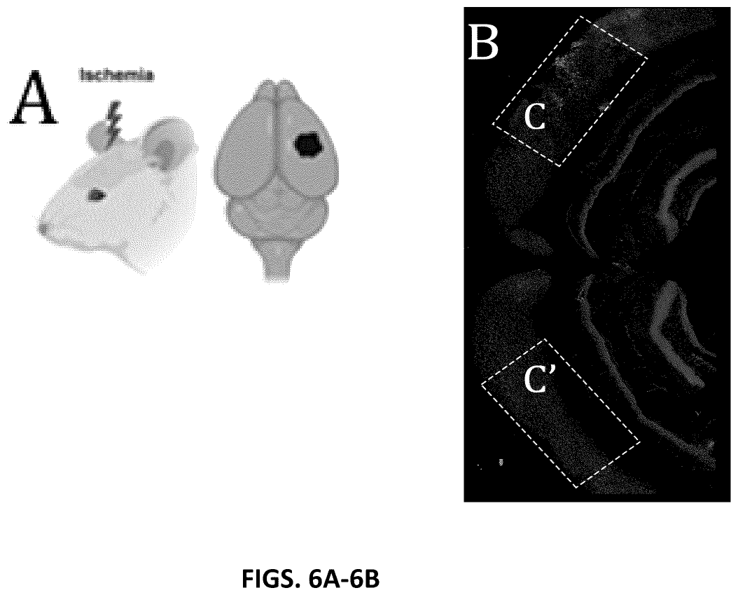

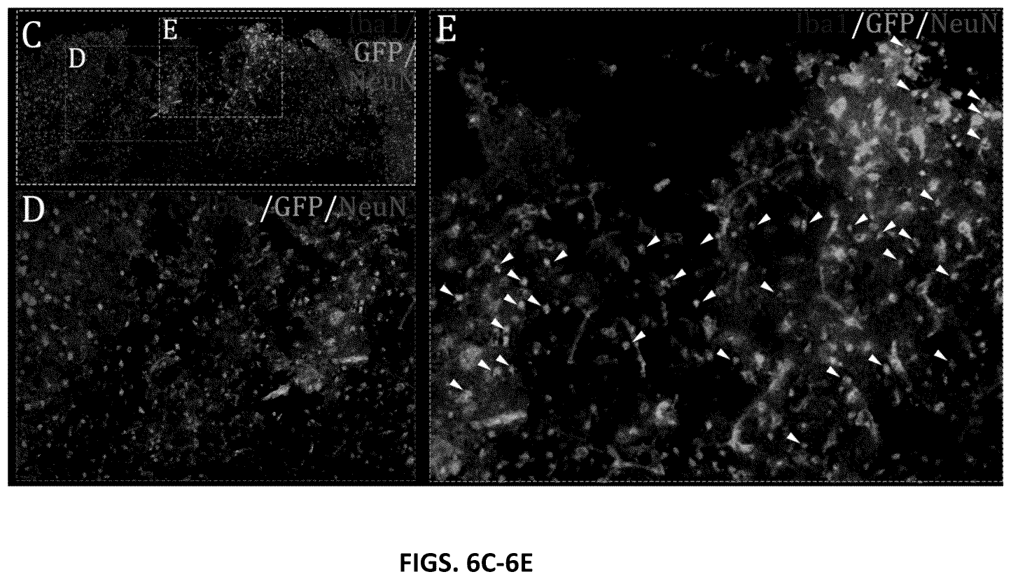

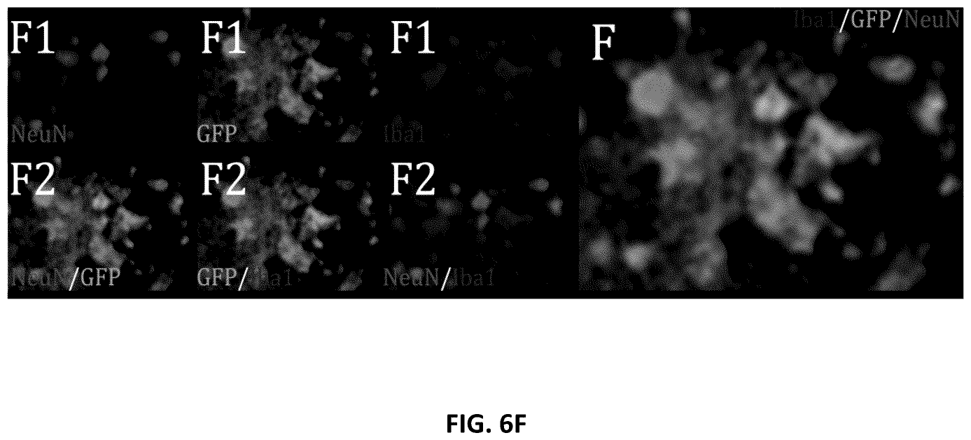

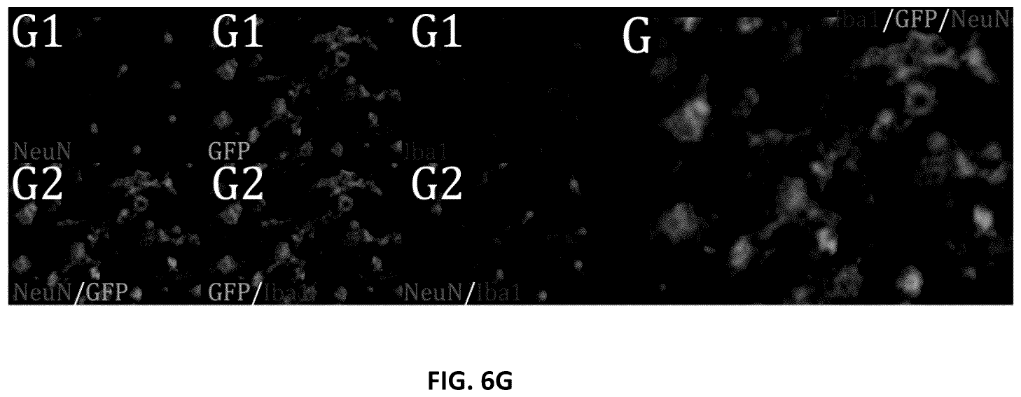

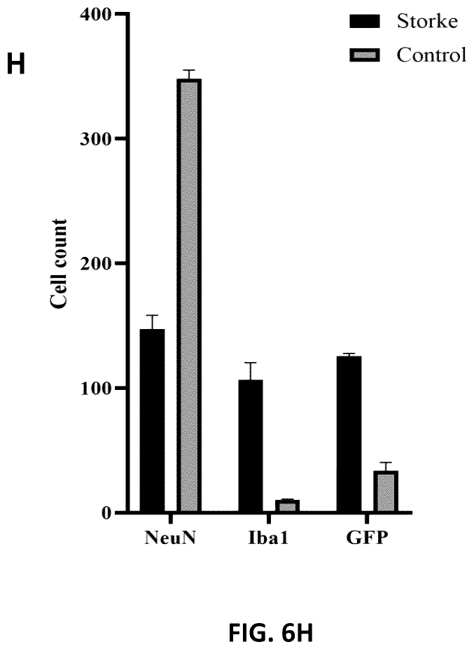

In order to study the potential of eaEV targeting a confined inflammatory site, a photothrombotic stroke model was generated by inducing an ischemic damage in a small area within the mice cortex via photo-activation of the light-sensitive Rose Bengal dye, injected intraperitoneally (FIG. 6A). Leukocyte EVs (3E+07 total EVs per gram of body weight) were IV injected 24 hours after the stroke induction, and brains were collected 2 days after the EV administration A higher level of GFP expression in NeuN+ neurons of the injured brain region marked by increased Iba1 (a microglial/MΦ marker to evaluate the level of inflammation, FIG. 6B) suggested that leukocyte derived eaEV delivered mRNA across the BBB and enriched at inflammatory sites upon injury (FIG. 6C-G). In the injured area, neurons were damaged, decreasing the number of NeuN+ cells (FIG. 6H). More Iba1+ cells were counted as the site is highly inflammatory, attracting microglia and infiltrated macrophages (FIG. 6H). The number of GFP+ cells was increased (FIG. 6H). Thus, leukocyte eaEV can deliver mRNA across the BBB locally targeting injury-induced inflammation without affecting healthy cells in the same brain.

In conclusion, a safe and effective mRNA drug carrier with high cargo loading and delivery efficiency was engineered. Methods to produce and isolate eaEV were optimized, providing detailed profiling of the cargo loading and transfer efficacy. Furthermore, the ability of eaEV to cross the BBB and deliver mRNA into animal models for neuroinflammation was shown, indicating the potential of eaEV to be used in novel therapies for inflammatory conditions in the CNS.

### Example 7. Leukocyte Derived A5U-eaEV can Deliver mRNA Deep Penetrating Solid Tumors

In addition to neuroinflammation, eaEV was tested the tumor inflammatory microenvironment. Chronic inflammation and increased permeability are also prominent features of cancer, and both extrinsic and intrinsic factors can trigger an inflammatory response in the tumor microenvironment (TME), such as imbalanced immune regulation, carcinogen exposure, as well as genetic alterations leading to the activation of oncogenes or the loss of tumor suppressors. TME vasculature often possesses aberrant morphology associated with a leaky, chaotically organized, immature, thin-walled, and ill-perfused network of vessels, caused by poor pericyte coverage and supportive basement membrane disruption of endothelial cells. Therefore, it was hypothesized that leukocyte derived eaEV can deliver mRNA into the TME.

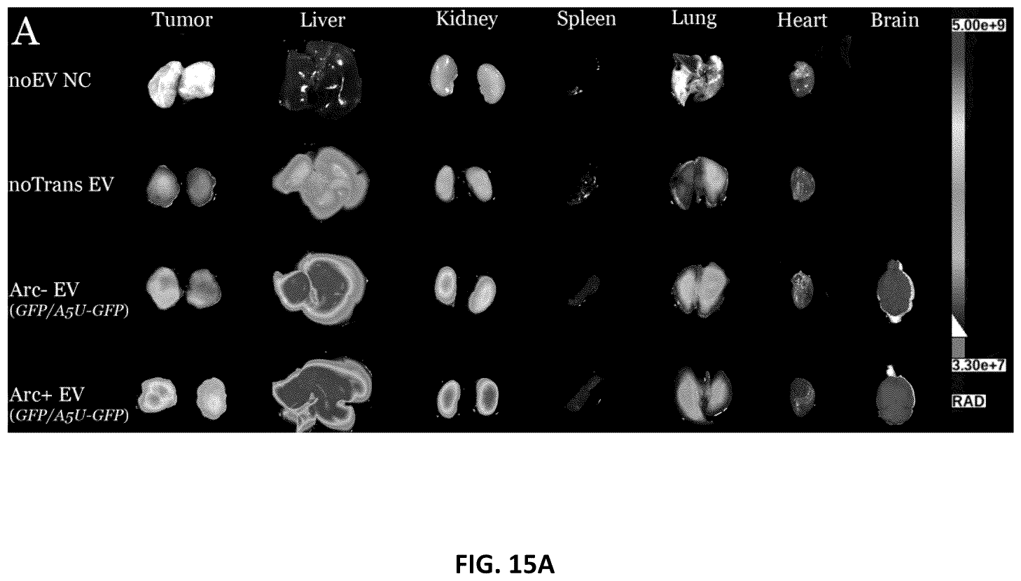

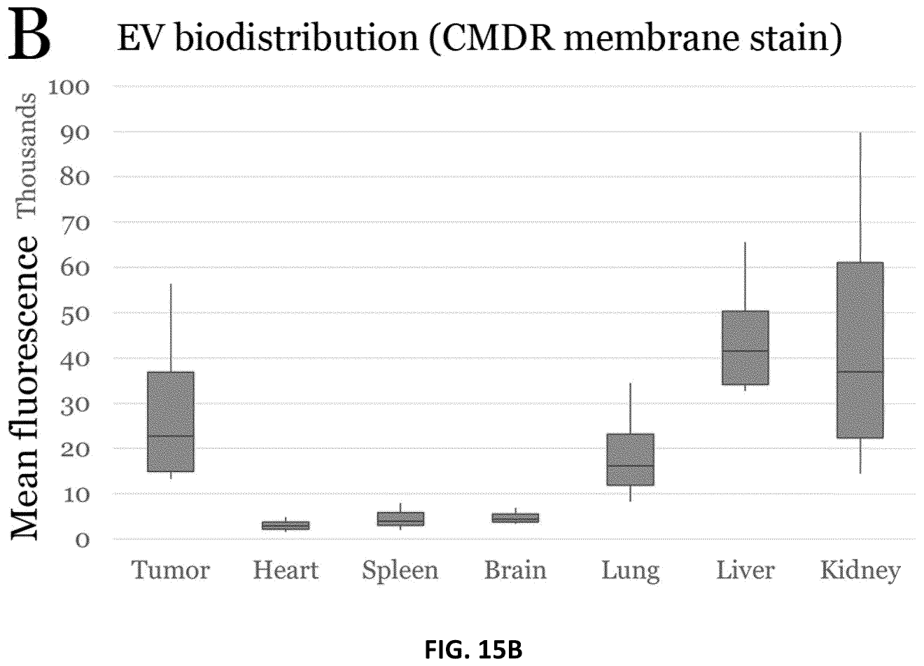

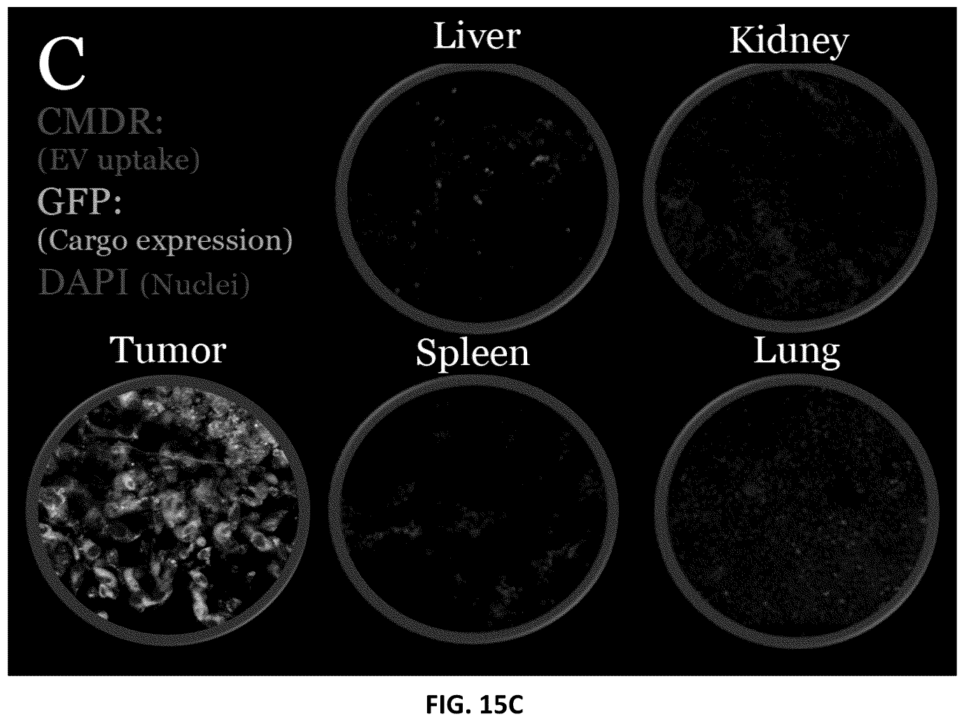

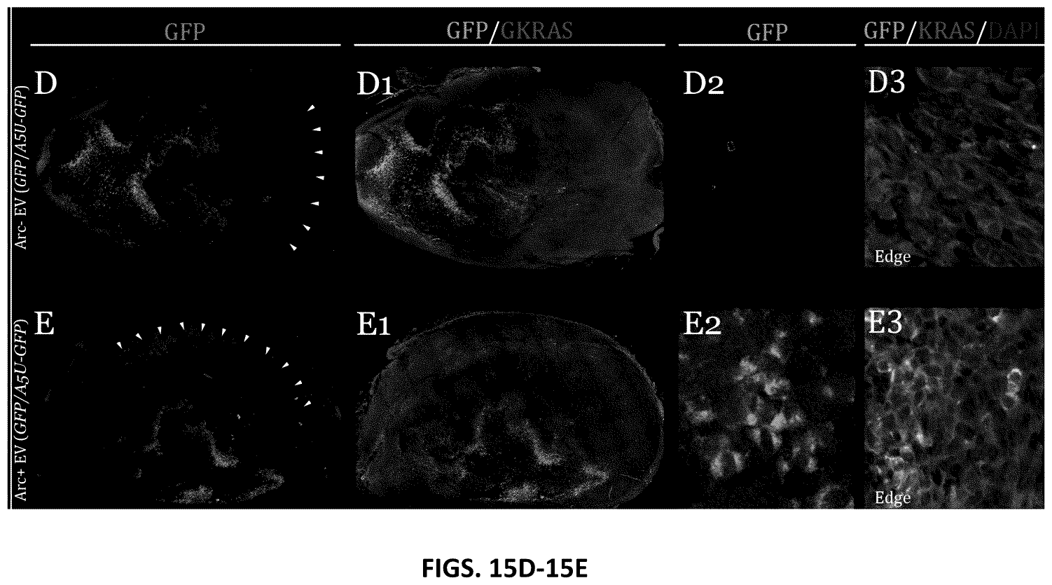

First a triple negative breast cancer (TNBC) mouse model was generated via subcutaneous injection of MDAMB231 cells (3× 106) into mice (4-6 weeks female NIH-III nude mouse). When tumor volume reached ˜1,000 mm3 (˜2 weeks), control and eaEVs carrying GFP or A5U-GFP cargo mRNAs were IV injected. EVs were produced, isolated, characterized and quantified as described above, with the same amount of total EVs injected per gram of body weight. To study the in vivo biodistribution of eaEVs in the TME, organs were collected 3 days after the IV injection for IVIS analysis. Transcardial perfusion was performed before dissecting the organs to remove any residual eaEVs in the circulation. Here, non-fluorescent mRNAs and labeled total EVs with the CMDR membrane stain were transfected. Significantly increased CMDR signal in the tumor by Arc+ EVs was observed, whereas other organs showing similar CMDR levels (FIG. 15A). In this experiment, the distribution of CMDR+ EVs could not be distinguished from that of the dye itself. Animals receiving leukocyte eaEVs showed comparable amount of CMDR in the tumor as in liver and kidney, both of which are involved in lipid metabolism (FIG. 15B). Despite having ample CMDR accumulation, a significantly higher level of GFP was expressed in the tumor shown by confocal imaging at cellular resolution (FIG. 15C). Then, the translation of cargo mRNA was examined in the tumors, which were dissected, fixed, thick-sectioned, cleared and IHC stained by K-Ras before CLSM. The results suggest that eaEV penetrated deep into the tumor tissue to express the mRNA cargo, whereases control EVs only distributed in the vicinity of duct (FIG. 15D-E). Altogether, these results demonstrated deep tumor penetration of mRNA loaded eaEV enabling efficient mRNA uptake and translation.

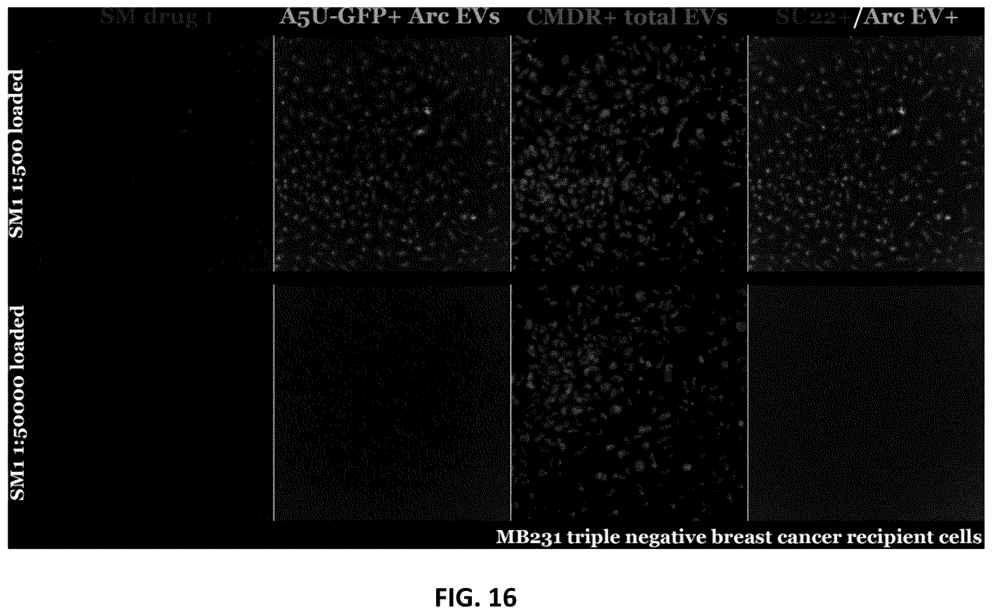

Since the addition of Arc improves tumor targeting, anti-tumor small molecules drugs were loaded into eaEV to test the potential of eaEV in anti-tumor therapies. Three methods were used to load these drugs: (1) small molecule drugs are added the culture media of donor cells after DNA/RNA transfection of the capsid and cargo constructs; (2) small molecule drugs are incubated with purified EVs from the donor cell culture; (3) small molecule drugs are loaded into purified eaEVs by low power sonication (six cycles of 30 s on/off for a total of 3 min, with 2 min cooling). Small molecule drugs were successfully loaded and delivered into recipient triple negative breast cancer cells (FIG. 16). Therefore, eaEV's tumor targeting feature can be used to deliver small molecule drugs deep into the tumor.

In conclusion, a safe and effective mRNA drug carrier with high cargo loading and delivery efficiency was engineered. The methods to produce and isolate eaEV were optimized, providing detailed profiling of the cargo loading and transfer efficacy. Moreover, eaEV enabled deep tumor penetration and efficient mRNA delivery in an animal model for breast cancer, demonstrating its potential in novel therapies for cancer.

TABLE 1
List of Sequences

| SEQ ID | | | |
|---|---|---|---|
| NO: | Species | Type | Description |
| 1 | Homo sapien | Nucleic Acid | Human Arc 5′ UTR sequence (NCBI Gene ID #23237) |
| 2 | Mus musculus | Nucleic Acid | Mouse Arc 5′ UTR sequence (NCBI Gene ID #11838) |
| 3 | Rattus morvegicus | Nucleic Acid | Rat Arc 5′ UTR sequence (NCBI Gene ID #54323) |
| 4 | Drosophila melanogaster | Nucleic Acid | Drosophila dArc1 5′ UTR sequence (NCBI Gene ID |
| 5 | Homo sapien | Nucleic Acid | Human Arc 3′ UTR sequence (NCBI Gene ID #23237) |
| 6 | Mus musculus | Nucleic Acid | Mouse Arc 3′ UTR sequence (NCBI Gene ID #11838) |
| 7 | Rattus morvegicus | Nucleic Acid | Rat Arc 3′ UTR sequence (NCBI Gene ID #54323) |
| 8 | Drosophila melanogaster | Nucleic Acid | Drosophila dArc1 3′ UTR sequence (NCBI Gene ID |
| 9 | Homo sapien | Nucleic Acid | Human Arc mRNA sequence (NCBI Gene ID #23237) |
| 10 | Mus musculus | Nucleic Acid | Mouse Arc mRNA sequence (NCBI Gene ID #11838) |
| 11 | Rattus morvegicus | Nucleic Acid | Rat Arc mRNA sequence (NCBI Gene ID #54323) |
| 12 | Drosophila melanogaster | Nucleic Acid | Drosophila Arc mRNA sequence (NCBI Gene ID #36595) |
| 13 | Homo sapien | Amino Acid | Human Arc amino acid sequence (NCBI Gene ID #23237) |
| 14 | Mus musculus | Amino Acid | Mouse Arc amino acid sequence (NCBI Gene ID #11838) |
| 15 | Rattus morvegicus | Amino Acid | Rat Arc amino acid sequence (NCBI Gene ID #54323) |
| 16 | Drosophila melanogaster | Amino Acid | Drosophila Arc amino acid sequence (NCBI Gene ID #36595) |
| 17 | Gallus gallus | Nucleic Acid | Arc NCBI Gene ID # 395075 |
| 18 | Xenopus tropicalis | Nucleic Acid | Arc NCBI Gene ID # 100271754 |
| 19 | Taeniopygia guttata | Nucleic Acid | Arc NCBI Gene ID # 752001 |
| 20 | Macaca mulatta | Nucleic Acid | Arc NCBI Gene ID # 702690 |
| 21 | Bos taurus | Nucleic Acid | Arc NCBI Gene ID # 519403 |
| 22 | Pan troglodytes | Nucleic Acid | Arc NCBI Gene ID # 472874 |
| 23 | Callithrix jacchus | Nucleic Acid | Arc NCBI Gene ID # 100387688 |
| 24 | Columba livia | Nucleic Acid | Arc NCBI Gene ID # 102091501 |
| 25 | Chelonia mydas | Nucleic Acid | Arc NCBI Gene ID # 102941153 |
| 26 | Mesocricetus auratus | Nucleic Acid | Arc NCBI Gene ID # 101841579 |
| 27 | Equus caballus | Nucleic Acid | Arc NCBI Gene ID # 100146865 |
| 28 | Marmota monax | Nucleic Acid | Arc NCBI Gene ID # 124095804 |
| 29 | Meles meles | Nucleic Acid | Arc NCBI Gene ID # 123941397 |
| 30 | Mirounga angustirostris | Nucleic Acid | Arc NCBI Gene ID # 123858672 |
| 31 | Phyllostomus hastatus | Nucleic Acid | Arc NCBI Gene ID # 123833165 |
| 32 | Lemur catta | Nucleic Acid | Arc NCBI Gene ID # 123644519 |
| 33 | Leopardus geoffroyi | Nucleic Acid | Arc NCBI Gene ID # 123585384 |
| 34 | Mauremys mutica | Nucleic Acid | Arc NCBI Gene ID # 123365322 |
| 35 | Equus asinus | Nucleic Acid | Arc NCBI Gene ID # 123275551 |
| 36 | Gracilinanus agilis | Nucleic Acid | Arc NCBI Gene ID # 123231978 |
| 37 | Varanus komodoensis | Nucleic Acid | Arc NCBI Gene ID # 123035313 |
| 38 | Bufo gargarizans | Nucleic Acid | Arc NCBI Gene ID # 122939220 |
| 39 | Neogale vison | Nucleic Acid | Arc NCBI Gene ID # 122905503 |
| 40 | Dromiciops gliroides | Nucleic Acid | Arc NCBI Gene ID # 122736597 |
| 41 | Cervus elaphus | Nucleic Acid | Arc NCBI Gene ID # 122679325 |
| 42 | Prionailurus bengalensis | Nucleic Acid | Arc NCBI Gene ID # 122495866 |
| 43 | Cervus canadensis | Nucleic Acid | Arc NCBI Gene ID # 122451035 |
| 44 | Panthera tigris | Nucleic Acid | Arc NCBI Gene ID # 122234928 |
| 45 | Panthera leo | Nucleic Acid | Arc NCBI Gene ID # 122210737 |
| 46 | Lagopus leucura | Nucleic Acid | Arc NCBI Gene ID # 122178992 |
| 47 | Centrocercus urophasianus | Nucleic Acid | Arc NCBI Gene ID # 122155226 |
| 48 | Dipodomys spectabilis | Nucleic Acid | Arc NCBI Gene ID # 122125347 |
| 49 | Sceloporus undulatus | Nucleic Acid | Arc NCBI Gene ID # 121930018 |
| 50 | Corvus kubaryi | Nucleic Acid | Arc NCBI Gene ID # 121668452 |
| 51 | Vulpes lagopus | Nucleic Acid | Arc NCBI Gene ID # 121498529 |
| 52 | Microtus oregoni | Nucleic Acid | Arc NCBI Gene ID # 121461017 |
| 53 | Pyrgilauda ruficollis | Nucleic Acid | Arc NCBI Gene ID # 121349289 |
| 54 | Onychostruthus taczanowskii | Nucleic Acid | Arc NCBI Gene ID # 121335950 |
| 55 | Ochotona curzoniae | Nucleic Acid | Arc NCBI Gene ID # 121161888 |
| 56 | Falco naumanni | Nucleic Acid | Arc NCBI Gene ID # 121085152 |
| 57 | Cygnus olor | Nucleic Acid | Arc NCBI Gene ID # 121065018 |
| 58 | Puma yagouaroundi | Nucleic Acid | Arc NCBI Gene ID # 121025873 |
| 59 | Bufo bufo | Nucleic Acid | Arc NCBI Gene ID # 121002044 |
| 60 | Rana temporaria | Nucleic Acid | Arc NCBI Gene ID # 120939740 |
| 61 | Oryx dammah | Nucleic Acid | Arc NCBI Gene ID # 120878789 |
| 62 | Hirundo rustica | Nucleic Acid | Arc NCBI Gene ID # 120753894 |
| 63 | Pteropus giganteus | Nucleic Acid | Arc NCBI Gene ID # 120588762 |
| 64 | Passer montanus | Nucleic Acid | Arc NCBI Gene ID # 120504749 |
| 65 | Mauremys reevesii | Nucleic Acid | Arc NCBI Gene ID # 120396602 |
| 66 | Crotalus tigris | Nucleic Acid | Arc NCBI Gene ID # 120314226 |
| 67 | Hyaena hyaena | Nucleic Acid | Arc NCBI Gene ID # 120232326 |
| 68 | Tachyglossus aculeatus | Nucleic Acid | Arc NCBI Gene ID # 119940264 |
| 69 | Dermochelys coriacea | Nucleic Acid | Arc NCBI Gene ID # 119850344 |
| 70 | Arvicola amphibius | Nucleic Acid | Arc NCBI Gene ID # 119822193 |
| 71 | Motacilla alba alba | Nucleic Acid | Arc NCBI Gene ID # 119698080 |
| 72 | Talpa occidentalis | Nucleic Acid | Arc NCBI Gene ID # 119232354 |
| 73 | Falco rusticolus | Nucleic Acid | Arc NCBI Gene ID # 119145010 |
| 74 | Artibeus jamaicensis | Nucleic Acid | Arc NCBI Gene ID # 119064763 |
| 75 | Sturnira hondurensis | Nucleic Acid | Arc NCBI Gene ID # 118988716 |
| 76 | Manis pentadactyla | Nucleic Acid | Arc NCBI Gene ID # 118930717 |
| 77 | Balaenoptera musculus | Nucleic Acid | Arc NCBI Gene ID # 118883868 |
| 78 | Trichosurus vulpecula | Nucleic Acid | Arc NCBI Gene ID # 118828797 |
| 79 | Pipistrellus kuhlii | Nucleic Acid | Arc NCBI Gene ID # 118718627 |
| 80 | Molothrus ater | Nucleic Acid | Arc NCBI Gene ID # 118701631 |
| 81 | Myotis myotis | Nucleic Acid | Arc NCBI Gene ID # 118670582 |
| 82 | Molossus molossus | Nucleic Acid | Arc NCBI Gene ID # 118643339 |
| 83 | Onychomys torridus | Nucleic Acid | Arc NCBI Gene ID # 118597471 |
| 84 | Halichoerus grypus | Nucleic Acid | Arc NCBI Gene ID # 118548735 |
| 85 | Cygnus atratus | Nucleic Acid | Arc NCBI Gene ID # 118257931 |
| 86 | Oxyura jamaicensis | Nucleic Acid | Arc NCBI Gene ID # 118162701 |
| 87 | Zootoca vivipara | Nucleic Acid | Arc NCBI Gene ID # 118090548 |
| 88 | Mirounga leonina | Nucleic Acid | Arc NCBI Gene ID # 118015255 |
| 89 | Trachemys scripta elegans | Nucleic Acid | Arc NCBI Gene ID # 117873854 |
| 90 | Arvicanthis niloticus | Nucleic Acid | Arc NCBI Gene ID # 117719109 |
| 91 | Pantherophis guttatus | Nucleic Acid | Arc NCBI Gene ID # 117666901 |
| 92 | Trachypithecus francoisi | Nucleic Acid | Arc NCBI Gene ID # 117079334 |
| 93 | Lacerta agilis | Nucleic Acid | Arc NCBI Gene ID # 117050329 |
| 94 | Rhinolophus ferrumequinum | Nucleic Acid | Arc NCBI Gene ID # 117033761 |
| 95 | Catharus ustulatus | Nucleic Acid | Arc NCBI Gene ID # 117004964 |
| 96 | Rattus rattus | Nucleic Acid | Arc NCBI Gene ID # 116912504 |
| 97 | Lontra canadensis | Nucleic Acid | Arc NCBI Gene ID # 116877009 |
| 98 | Chelonoidis abingdonii | Nucleic Acid | Arc NCBI Gene ID # 116815155 |
| 99 | Hylobates moloch | Nucleic Acid | Arc NCBI Gene ID # 116813636 |
| 100 | Chiroxiphia lanceolata | Nucleic Acid | Arc NCBI Gene ID # 116798476 |
| 101 | Phocoena sinus | Nucleic Acid | Arc NCBI Gene ID # 116742157 |
| 102 | Phoca vitulina | Nucleic Acid | Arc NCBI Gene ID # 116639541 |
| 103 | Mustela erminea | Nucleic Acid | Arc NCBI Gene ID # 116575408 |
| 104 | Sapajus apella | Nucleic Acid | Arc NCBI Gene ID # 116534300 |
| 105 | Thamnophis elegans | Nucleic Acid | Arc NCBI Gene ID # 116512059 |
| 106 | Aythya fuligula | Nucleic Acid | Arc NCBI Gene ID # 116486828 |
| 107 | Corvus moneduloides | Nucleic Acid | Arc NCBI Gene ID # 116435325 |
| 108 | Phasianus colchicus | Nucleic Acid | Arc NCBI Gene ID # 116227526 |
| 109 | Mastomys coucha | Nucleic Acid | Arc NCBI Gene ID # 116081111 |
| 110 | Camarhynchus parvulus | Nucleic Acid | Arc NCBI Gene ID # 115900969 |
| 111 | Globicephala melas | Nucleic Acid | Arc NCBI Gene ID # 115863283 |
| 112 | Gopherus evgoodei | Nucleic Acid | Arc NCBI Gene ID # 115647144 |
| 113 | Strigops habroptila | Nucleic Acid | Arc NCBI Gene ID # 115617032 |
| 114 | Anas platyrhynchos | Amino Acid | Peking Duck Arc amino acid sequence |
| | | | (NCBI Gene ID # 101790723) |
| 115 | Gallus gallus | Amino Acid | Arc NCBI Gene ID # 395075 |
| 116 | Xenopus tropicalis | Amino Acid | Arc NCBI Gene ID # 100271754 |
| 117 | Taeniopygia guttata | Amino Acid | Arc NCBI Gene ID # 752001 |
| 118 | Macaca mulatta | Amino Acid | Arc NCBI Gene ID # 702690 |
| 119 | Bos taurus | Amino Acid | Arc NCBI Gene ID # 519403 |
| 120 | Pan troglodytes | Amino Acid | Arc NCBI Gene ID # 472874 |
| 121 | Callithrix jacchus | Amino Acid | Arc NCBI Gene ID # 100387688 |
| 122 | Columba livia | Amino Acid | Arc NCBI Gene ID # 102091501 |
| 123 | Chelonia mydas | Amino Acid | Arc NCBI Gene ID # 102941153 |
| 124 | Mesocricetus auratus | Amino Acid | Arc NCBI Gene ID # 101841579 |
| 125 | Equus caballus | Amino Acid | Arc NCBI Gene ID # 100146865 |
| 126 | Marmota monax | Amino Acid | Arc NCBI Gene ID # 124095804 |
| 127 | Meles meles | Amino Acid | Arc NCBI Gene ID # 123941397 |
| 128 | Mirounga angustirostris | Amino Acid | Arc NCBI Gene ID # 123858672 |
| 129 | Phyllostomus hastatus | Amino Acid | Arc NCBI Gene ID # 123833165 |
| 130 | Lemur catta | Amino Acid | Arc NCBI Gene ID # 123644519 |
| 131 | Leopardus geoffroyi | Amino Acid | Arc NCBI Gene ID # 123585384 |
| 132 | Mauremys mutica | Amino Acid | Arc NCBI Gene ID # 123365322 |
| 133 | Equus asinus | Amino Acid | Arc NCBI Gene ID # 123275551 |
| 134 | Gracilinanus agilis | Amino Acid | Arc NCBI Gene ID # 123231978 |
| 135 | Varanus komodoensis | Amino Acid | Arc NCBI Gene ID # 123035313 |
| 136 | Bufo gargarizans | Amino Acid | Arc NCBI Gene ID # 122939220 |
| 137 | Neogale vison | Amino Acid | Arc NCBI Gene ID # 122905503 |
| 138 | Dromiciops gliroides | Amino Acid | Arc NCBI Gene ID # 122736597 |
| 139 | Cervus elaphus | Amino Acid | Arc NCBI Gene ID # 122679325 |
| 140 | Prionailurus bengalensis | Amino Acid | Arc NCBI Gene ID # 122495866 |
| 141 | Cervus canadensis | Amino Acid | Arc NCBI Gene ID # 122451035 |
| 142 | Panthera tigris | Amino Acid | Arc NCBI Gene ID # 122234928 |
| 143 | Panthera leo | Amino Acid | Arc NCBI Gene ID # 122210737 |
| 144 | Lagopus leucura | Amino Acid | Arc NCBI Gene ID # 122178992 |
| 145 | Centrocercus urophasianus | Amino Acid | Arc NCBI Gene ID # 122155226 |
| 146 | Dipodomys spectabilis | Amino Acid | Arc NCBI Gene ID # 122125347 |
| 147 | Dipodomys spectabilis | Amino Acid | Arc NCBI Gene ID # 122125347 |
| 148 | Sceloporus undulatus | Amino Acid | Arc NCBI Gene ID # 121930018 |
| 149 | Corvus kubaryi | Amino Acid | Arc NCBI Gene ID # 121668452 |
| 150 | Vulpes lagopus | Amino Acid | Arc NCBI Gene ID # |
| 151 | Microtus oregoni | Amino Acid | Arc NCBI Gene ID # 121461017 |
| 152 | Pyrgilauda ruficollis | Amino Acid | Arc NCBI Gene ID # 121349289 |
| 153 | Onychostruthus taczanowskii | Amino Acid | Arc NCBI Gene ID # 121335950 |
| 154 | Ochotona curzoniae | Amino Acid | Arc NCBI Gene ID # 121161888 |
| 155 | Falco naumanni | Amino Acid | Arc NCBI Gene ID # 121085152 |
| 156 | Cygnus olor | Amino Acid | Arc NCBI Gene ID # 121065018 |
| 157 | Puma yagouaroundi | Amino Acid | Arc NCBI Gene ID # 121025873 |
| 158 | Bufo bufo | Amino Acid | Arc NCBI Gene ID # 121002044 |
| 159 | Rana temporaria | Amino Acid | Arc NCBI Gene ID # 120939740 |
| 160 | Oryx dammah | Amino Acid | Arc NCBI Gene ID # 120878789 |
| 161 | Hirundo rustica | Amino Acid | Arc NCBI Gene ID # 120753894 |
| 162 | Pteropus giganteus | Amino Acid | Arc NCBI Gene ID # 120588762 |
| 163 | Passer montanus | Amino Acid | Arc NCBI Gene ID # 120504749 |
| 164 | Mauremys reevesii | Amino Acid | Arc NCBI Gene ID # 120396602 |
| 165 | Crotalus tigris | Amino Acid | Arc NCBI Gene ID # 120314226 |
| 166 | Hyaena hyaena | Amino Acid | Arc NCBI Gene ID # 120232326 |
| 167 | Tachyglossus aculeatus | Amino Acid | Arc NCBI Gene ID # 119940264 |
| 168 | Dermochelys coriacea | Amino Acid | Arc NCBI Gene ID # 119850344 |
| 169 | Arvicola amphibius | Amino Acid | Arc NCBI Gene ID # 119822193 |
| 170 | Motacilla alba alba | Amino Acid | Arc NCBI Gene ID # 119698080 |
| 171 | Talpa occidentalis | Amino Acid | Arc NCBI Gene ID # 119232354 |
| 172 | Falco rusticolus | Amino Acid | Arc NCBI Gene ID # 119145010 |
| 173 | Artibeus jamaicensis | Amino Acid | Arc NCBI Gene ID # 119064763 |
| 174 | Sturnira hondurensis | Amino Acid | Arc NCBI Gene ID # 118988716 |
| 175 | Manis pentadactyla | Amino Acid | Arc NCBI Gene ID # 118930717 |
| 176 | Balaenoptera musculus | Amino Acid | Arc NCBI Gene ID # 118883868 |
| 177 | Trichosurus vulpecula | Amino Acid | Arc NCBI Gene ID # 118828797 |
| 178 | Pipistrellus kuhlii | Amino Acid | Arc NCBI Gene ID # 118718627 |
| 179 | Molothrus ater | Amino Acid | Arc NCBI Gene ID # 118701631 |
| 180 | Myotis myotis | Amino Acid | Arc NCBI Gene ID # 118670582 |
| 181 | Molossus molossus | Amino Acid | Arc NCBI Gene ID # 118643339 |
| 182 | Onychomys torridus | Amino Acid | Arc NCBI Gene ID # 118597471 |
| 183 | Halichoerus grypus | Amino Acid | Arc NCBI Gene ID # 118548735 |
| 184 | Cygnus atratus | Amino Acid | Arc NCBI Gene ID # 118257931 |
| 185 | Oxyura jamaicensis | Amino Acid | Arc NCBI Gene ID # 118162701 |
| 186 | Zootoca vivipara | Amino Acid | Arc NCBI Gene ID # 118090548 |
| 187 | Mirounga leonina | Amino Acid | Arc NCBI Gene ID # 118015255 |
| 188 | Trachemys scripta elegans | Amino Acid | Arc NCBI Gene ID # 117873854 |
| 189 | Arvicanthis niloticus | Amino Acid | Arc NCBI Gene ID # 117719109 |
| 190 | Pantherophis guttatus | Amino Acid | Arc NCBI Gene ID # 117666901 |
| 191 | Trachypithecus francoisi | Amino Acid | Arc NCBI Gene ID # 117079334 |
| 192 | Lacerta agilis | Amino Acid | Arc NCBI Gene ID # 117050329 |
| 193 | Rhinolophus ferrumequinum | Amino Acid | Arc NCBI Gene ID # 117033761 |
| 194 | Catharus ustulatus | Amino Acid | Arc NCBI Gene ID # 117004964 |
| 195 | Rattus rattus | Amino Acid | Arc NCBI Gene ID # 116912504 |
| 196 | Lontra canadensis | Amino Acid | Arc NCBI Gene ID # 116877009 |
| 197 | Chelonoidis abingdonii | Amino Acid | Arc NCBI Gene ID # 116815155 |
| 198 | Hylobates moloch | Amino Acid | Arc NCBI Gene ID # 116813636 |
| 199 | Chiroxiphia lanceolata | Amino Acid | Arc NCBI Gene ID # 116798476 |
| 200 | Phocoena sinus | Amino Acid | Arc NCBI Gene ID # 116742157 |
| 201 | Phoca vitulina | Amino Acid | Arc NCBI Gene ID # 116639541 |
| 202 | Mustela erminea | Amino Acid | Arc NCBI Gene ID # 116575408 |
| 203 | Sapajus apella | Amino Acid | Arc NCBI Gene ID # 116534300 |
| 204 | Thamnophis elegans | Amino Acid | Arc NCBI Gene ID # 116512059 |
| 205 | Aythya fuligula | Amino Acid | Arc NCBI Gene ID # 116486828 |
| 206 | Corvus moneduloides | Amino Acid | Arc NCBI Gene ID # 116435325 |
| 207 | Phasianus colchicus | Amino Acid | Arc NCBI Gene ID # 116227526 |
| 208 | Mastomys coucha | Amino Acid | Arc NCBI Gene ID # 116081111 |
| 209 | Camarhynchus parvulus | Amino Acid | Arc NCBI Gene ID # 115900969 |
| 210 | Globicephala melas | Amino Acid | Arc NCBI Gene ID # 115863283 |
| 211 | Gopherus evgoodei | Amino Acid | Arc NCBI Gene ID # 115647144 |
| 212 | Strigops habroptila | Amino Acid | Arc NCBI Gene ID # 115617032 |

What is claimed is:

1. An RNA transcript composition comprising a cargo mRNA which comprises an Arc 5′UTR sequence.

2. The RNA transcript composition of claim 1, further comprising an Arc mRNA.

3. The composition of claim 1 or 2, wherein the Arc 5′UTR sequence comprises an Arc 5′UTR sequence from a mammal.

4. The composition of any one of claims 1-3, wherein the Arc 5′UTR sequence comprises an Arc 5′UTR sequence from human, mouse, or rat.

5. The composition of claim 1 or 2, wherein the Arc 5′UTR sequence comprises an Arc 5′UTR sequence from drosophila.

6. The composition of any one of claims 1-5, wherein the Arc 5′UTR sequence comprises a sequence having at least 80% sequence identity, at least 85% sequence identity, at least 90% sequence identity, at least 95% sequence identity, at least 98% sequence identity, or at least 99% sequence identity to any one of SEQ ID NOS: 1-4.

7. The composition of any one of claims 1-6, wherein the cargo mRNA further comprises a poly(A) signal.

8. The composition of any one of claims 1-7, wherein the cargo mRNA encodes a therapeutic protein.

9. The composition of any one of claims 1-7, wherein the cargo mRNA encodes a peptide, an enzyme, a cytokine, a hormone, a growth factor, an antigen, an antibody, a portion of an antibody, a clotting factor, a regulatory protein, a signaling protein, a transcription protein, and/or a receptor.

10. The composition of any one of claims 1-7, wherein the cargo mRNA encodes a fluorescent protein, a bioluminescent protein, and/or a recombinase reporter.

11. The composition of any one of claims 1-10, wherein the Arc mRNA comprises an Arc 3′UTR sequence.

12. The composition of any one of claims 1-11, wherein the Arc mRNA comprises an Arc 3′UTR sequence from a mammal.

13. The composition of claim 12, wherein the mammal is human, mouse, or rat.

14. The composition of any one of claims 1-11, wherein the Arc 3′UTR sequence comprises an Arc 3′UTR sequence from drosophila.

15. The composition of any one of claims 11-14, wherein the Arc 3′UTR sequence comprises a sequence having at least 80% sequence identity, at least 85% sequence identity, at least 90% sequence identity, at least 95% sequence identity, at least 98% sequence identity, or at least 99% sequence identity to any one of SEQ ID NOS: 5-8.

16. The composition of any one of claims 1-15, wherein the Arc mRNA further comprises a poly(A) signal.

17. The composition of any one of claims 1-16, wherein the Arc mRNA encodes an Arc protein from a mammal.

18. The composition of claim 17, wherein the mammal is human, mouse, or rat.

19. The composition of any one of claims 1-16, wherein the Arc mRNA encodes an Arc protein from drosophila.

20. The composition of any one of claims 1-19, wherein the Arc mRNA comprises an Arc mRNA sequence from a mammal.

21. The composition of claim 20, wherein the mammal is human, mouse, or rat.

22. The composition of any one of claims 1-19, wherein the Arc mRNA comprises an Arc mRNA sequence from drosophila.

23. The composition of any one of claims 20-22, wherein the Arc mRNA comprises a nucleotide sequence having at least 80% sequence identity, at least 85% sequence identity, at least 90% sequence identity, at least 95% sequence identity, at least 98% sequence identity, or at least 99% sequence identity to any one of SEQ ID NOS: 9-12.

24. A recombinant system comprising a DNA encoding a cargo mRNA which comprises an Arc Arc 5′UTR sequence.

25. The system of claim 24, further comprising a second DNA encoding an Arc mRNA.

26. The system of claim 24 or 25, wherein the Arc 5′UTR sequence comprises an Arc 5′UTR sequence from a mammal.

27. The system of any one of claims 24-26, wherein the Arc 5′UTR sequence comprises an Arc 5′UTR sequence from human, mouse, or rat.

28. The system of claim 24 or 25, wherein the Arc 5′UTR sequence comprises an Arc 5′UTR sequence from drosophila.

29. The system of any one of claims 24-28, wherein the Arc 5′UTR sequence comprises a sequence having at least 80% sequence identity, at least 85% sequence identity, at least 90% sequence identity, at least 95% sequence identity, at least 98% sequence identity, or at least 99% sequence identity to any one of SEQ ID NOS: 1-4.

30. The system of any one of claims 24-29, wherein the cargo mRNA further comprises a poly(A) signal.

31. The system of any one of claims 24-30, wherein the cargo mRNA encodes a therapeutic protein.

32. The system of any one of claims 24-30, wherein the cargo mRNA encodes a peptide, an enzyme, a cytokine, a hormone, a growth factor, an antigen, an antibody, a portion of an antibody, a clotting factor, a regulatory protein, a signaling protein, a transcription protein, and/or a receptor.

33. The system of any one of claims 24-30, wherein the cargo mRNA encodes a fluorescent protein, a bioluminescent protein, and/or a recombinase reporter.

34. The system of any one of claims 24-33, wherein the Arc mRNA comprises an Arc 3′UTR sequence.

35. The system of any one of claims 24-34, wherein the Arc 3′UTR sequence comprises an Arc 3′UTR sequence from a mammal.

36. The system of any one of claims 24-35, wherein the Arc 3′UTR sequence comprises an Arc 3′UTR sequence is selected from human, mouse, or rat.

37. The system of any one of claims 24-33, wherein the Arc 3′UTR sequence comprises an Arc 3′UTR sequence from drosophila.

38. The system of any one of claims 34-37, wherein the Arc 3′UTR sequence comprises a sequence having at least 80% sequence identity, at least 85% sequence identity, at least 90% sequence identity, at least 95% sequence identity, at least 98% sequence identity, or at least 99% sequence identity to any one of SEQ ID NOS: 5-8.

39. The system of any one of claims 24-38, wherein the Arc mRNA further comprises a poly(A) signal.

40. The system of any one of claims 24-39, wherein the system comprises a single plasmid comprising the DNA encoding a cargo mRNA with an Arc 5′UTR sequence and the second DNA encoding an Arc mRNA.

41. The system of any one of claims 24-39, wherein the system comprises:
a first plasmid comprising the DNA encoding a cargo mRNA with an Arc 5′UTR sequence; and
a second plasmid comprising the second DNA encoding an Arc mRNA.

42. The system of any one of claims 40-41, wherein the plasmid(s) further comprises a heterologous DNA regulatory element.

43. The system of claim 42, wherein the heterologous DNA regulatory element comprises a promoter, an enhancer, a silencer, an insulator, or combinations thereof.

44. The system of any one of claims 24-43, wherein the Arc mRNA comprises an Arc mRNA sequence from a mammal.

45. The system of claim 44, wherein the mammal is human, mouse, or rat.

46. The system of any one of claims 24-43, wherein the Arc mRNA comprises an Arc mRNA sequence from drosophila.

47. The system of any one of claims 44-46, wherein the Arc 5′UTR sequence comprises a nucleotide sequence having at least 80% sequence identity, at least 85% sequence identity, at least 90% sequence identity, at least 95% sequence identity, at least 98% sequence identity, or at least 99% sequence identity to any one of SEQ ID NOS: 1-4.

48. The system of any one of claims 24-47, wherein the Arc mRNA encodes an Arc protein from a mammal.

49. The system of claim 48, wherein the mammal is human, mouse, or rat.

50. The system of any one of claims 24-47, wherein the Arc mRNA encodes an Arc protein from drosophila.

51. The system of any one of claims 24-50, wherein the Arc mRNA comprises an Arc mRNA sequence from a mammal.

52. The system of claim 51, wherein the mammal is human, mouse, or rat.

53. The system of any one of claims 24-50, wherein the Arc mRNA comprises an Arc mRNA sequence from drosophila.

54. The system of any one of claims 51-53, wherein the Arc mRNA comprises a nucleotide sequence having at least 80% sequence identity, at least 85% sequence identity, at least 90% sequence identity, at least 95% sequence identity, at least 98% sequence identity, or at least 99% sequence identity to any one of SEQ ID NOS: 9-12.

55. An extracellular vesicle comprising:
an Arc protein; and
a cargo mRNA comprising an Arc 5′UTR sequence.

56. The vesicle of claim 55, wherein the Arc 5′UTR sequence comprises an Arc 5′UTR sequence from a mammal.

57. The vesicle of claim 55 or claim 56, wherein the Arc 5′UTR sequence comprises an Arc 5′UTR sequence from human, mouse, or rat.

58. The vesicle of claim 55, wherein the Arc 5′UTR sequence comprises an Arc 5′UTR sequence from drosophila.

59. The vesicle of any one of claims 55-58, wherein the Arc 5′UTR sequence comprises a sequence having at least 80% sequence identity, at least 85% sequence identity, at least 90% sequence identity, at least 95% sequence identity, at least 98% sequence identity, or at least 99% sequence identity to any one of SEQ ID NOS: 1-4.

60. The vesicle of any one of claims 55-59, wherein the cargo mRNA further comprises a poly(A) signal.

61. The vesicle of any one of claims 55-60, wherein the cargo mRNA encodes a therapeutic protein.

62. The vesicle of any one of claims 55-60, wherein the cargo mRNA encodes a peptide, an enzyme, a cytokine, a hormone, a growth factor, an antigen, an antibody, a portion of an antibody, a clotting factor, a regulatory protein, a signaling protein, a transcription protein, and/or a receptor.

63. The vesicle of any one of claims 55-60, wherein the cargo mRNA encodes a fluorescent protein, a bioluminescent protein, and/or a recombinase reporter.

64. The vesicle of any one of claims 55-63, wherein the Arc protein comprises an Arc protein sequence from a mammal.

65. The vesicle of claim 64, wherein the mammal is human, mouse, or rat.

66. The vesicle of any one of claims 55-63, wherein the Arc protein comprises an Arc protein sequence from drosophila.

67. The vesicle of any one of claims 55-60, wherein the Arc protein comprises at least 80% sequence identity, at least 85% sequence identity, at least 90% sequence identity, at least 95% sequence identity, at least 98% sequence identity, or at least 99% sequence identity to any one of SEQ ID NOS: 13-16.

68. The vesicle of any one of claims 55-67, further comprising one or more small molecule drugs.

69. A method for producing extracellular vesicles, the method comprising:
(a) obtaining cells comprising an Arc mRNA and a cargo mRNA which comprises an Arc 5′ UTR;
(b) growing the cells in a media under conditions to express an Arc protein encoded by the Arc mRNA, wherein the cells produce extracellular vesicles comprising the Arc protein and the cargo mRNA with the Arc 5′UTR sequence; and
(c) isolating the extracellular vesicles from the media.

70. The method of claim 69, wherein the cells of step (a) are obtained by introducing into donor cells, a DNA construct which is transcribed into the Arc mRNA and a DNA construct which is transcribed into the cargo mRNA.

71. The method of claim 69, wherein the cells of step (a) are obtained by introducing into donor cells, the Arc mRNA and the cargo mRNA.

72. The method of any one of claims 69-71, wherein a recombinant construct is delivered in the form of DNA, RNA, or the combination of both.

73. The method of any one of claims 69-71, wherein the cell is a prokaryotic cell.

74. The method of any one of claims 69-71, wherein the cell is a eukaryotic cell.

75. The method of claim 74, wherein the cell is a mammalian cell.

76. The method of claim 75, wherein the cell is a human cell.

77. The method of claim 70 or claim 71, wherein the donor cells are selected from neural cells, epithelial cells, endothelial cells, hematopoietic cells, connective tissue cells, muscle cells, bone cells, cartilage cells, germline cells, adipocytes, stem cells, self-derived ex vivo differentiated cells, iPSC-derived ex vivo differentiated cells, cancer cells, and combinations thereof.

78. The method of claim 70 or claim 71, wherein the donor cells are leukocytes.

79. The method of claim 78, wherein the donor cells are self-derived ex vivo differentiated leukocytes.

80. The method of claim 79, wherein the donor cells are self-derived ex vivo differentiated monocytes, macrophages, dendritic cells, or combinations thereof.

81. The method of claim 77, wherein the donor cells are iPSC-derived ex vivo differentiated leukocytes.

82. The method of claim 78, wherein the donor cells are iPSC-derived ex vivo differentiated monocytes, macrophages, dendritic cells, or combinations thereof.

83. The method of any one of claims 69-82, wherein the cells comprising a nucleic acid construct as described in any of the preceding claims are prepared by transfecting cells with a nucleic acid construct as described in any of the preceding claims, wherein the transfection is carried out with polyethyleneimine (PEI) complexation, electroporation, cationic lipids complexation, lipid nanoparticle-mediated delivery, microinjection, and combinations thereof.

84. A method for delivering mRNA to a recipient cell, the method comprising:
obtaining an extracellular vesicle as described; and
contacting the recipient cell with the extracellular vesicle, wherein the extracellular vesicle fuses with the recipient cell, thereby delivering mRNA to the recipient cell.

85. The method of claim 84, wherein the contacting is performed in vitro.

86. The method of claim 84, wherein the contacting is performed in vivo.

87. The method of any one of claims 84-86, wherein the recipient cell is a mammalian cell.

88. The method of any one of claims 84-87, wherein the recipient cell comprises a hematopoietic cell, a non-hematopoietic cell, a stem cell, or combinations thereof.

89. The method of any one of claims 84-88, wherein the mRNA is delivered to a recipient cell to treat a disease, produce a protein, induce cell death, repress cell death, change cellular ageing, induce immune tolerance, modulate existing immune response, modify intracellular activity, modify cellular behavior, or combinations thereof.

90. A method for treating a subject in need thereof comprising:
obtaining extracellular vesicles as described; and
administering the extracellular vesicles to the subject.

91. The method of claim 90, wherein the extracellular vesicles are administered orally, rectally, intravenously, intramuscularly, subcutaneously, intrauterinely, cerebrovascularly, or intraventricularly.

92. The method of claim 90 or claim 91, wherein the extracellular vesicles comprise mRNAs of CRISPR-associated proteins and guide RNAs adapted for treatment of disease including a genetic disorder.

93. The method of any one of claims 90-92, wherein the extracellular vesicles are administered to the subject for treatment of neurodegeneration diseases, aging related disorders, brain tumors, an inflammatory condition, delivering RNAs specifically into inflammatory brain tissues crossing the blood brain barrier without affecting the healthy cells.

94. The method of claim 90 or claim 91, wherein the extracellular vesicles are adapted to deliver APOE4 RNA into the brain for the treatment of Alzheimer's disease.

95. The method of any one of claims 90-92, wherein the extracellular vesicles are administered for treatment of cancer, targeting tumor cells without affecting healthy tissues. Examples in this category include delivering IL 12 mRNA or OX40L mRNA for the treatment of solid tumors.

96. The method of claim 90 or claim 91, wherein the extracellular vesicles comprise mRNA corresponding to tumor associated antigens and wherein the extracellular vesicles are delivered as cancer vaccines for the treatment of cancer, including melanoma, colon cancer, gastrointestinal cancer, genitourinary cancer, hepatocellular cancer.

97. The method of claim 90 or claim 91, wherein the extracellular vesicles are delivered for the prevention and/or treatment of infectious diseases.

98. The method of claim 90 or claim 91, wherein the extracellular vesicles are delivered for the treatment of autoimmune diseases.

99. A method to deliver the construct of claim 1 to a recipient cell in vivo to produce an extracellular vesicle as described in claim 55 in vivo using an endogenous Arc.

100. The method of claim 99, wherein the vesicle is produced by the endogenous Arc in vivo.

101. The method of claim 99, wherein the construct is delivered in the form of DNA and/or RNA.

102. The method of claim 99, wherein the construct is delivered by a lipid nanoparticle, an exosome, a virus, and other gene delivery methods.
